(19) United States
(12) Patent Application Publication
(10) Pub. No.: US 2024/0117398 A1
(43) Pub. Date: Apr. 11, 2024

(54) PRODUCTION OF OLIGOSACCHARIDES COMPRISING LN3 AS CORE STRUCTURE IN HOST CELLS

(71) Applicant: INBIOSE N.V., Zwijnaarde (BE)

(72) Inventors: Sofie Aesaert, Zwijnaarde (BE); Joeri Beauprez, Zwijnaarde (BE); Gert Peters, Zwijnaarde (BE); Annelies Vercauteren, Zwijnaarde (BE)

(21) Appl. No.: 18/261,806

(22) PCT Filed: Jan. 20, 2022
(86) PCT No.: PCT/EP2022/051163

§ 371 Date: Jul. 17, 2023

(30) Foreign Application Priority Data
Jan. 20, 2021 (EP) 21152592.8

(51) Int. Cl. C12P 19/26; C07K 14/195; C12N 9/10; C12P 19/18

(52) U.S. Cl. CPC C12P 19/26; C07K 14/195; C12N 9/1051; C12P 19/18

(57) ABSTRACT

Described is a method of producing an oligosaccharide comprising a lacto-N-triose (LN3; GlcNAc-beta1,3-Gal-beta1,4-Glc) as a core trisaccharide by cultivation with a genetically modified cell, as well as the genetically modified cell used in the method. The genetically modified cell comprises at least one nucleic acid sequence coding for a galactoside beta-1,3-N-acetylglucosaminyltransferase and a glycosyltransferase involved in the synthesis of an oligosaccharide comprising LN3 as a core trisaccharide and at least one nucleic acid sequence expressing a membrane protein. Furthermore, the present invention provides for a purification of the oligosaccharide comprising LN3 as a core trisaccharide from the cultivation.

### CROSS-REFERENCE TO RELATED APPLICATIONS

This application is a national phase entry under 35 U.S.C. § 371 of International Patent Application PCT/EP2022/051163, filed Jan. 20, 2022, designating the United States of America and published as International Patent Publication WO 2022/157213 A1 on Jul. 28, 2022, which claims the benefit under Article 8 of the Patent Cooperation Treaty to European Patent Application Serial No. 21152592.8, filed Jan. 20, 2021.

### STATEMENT REGARDING ELECTRONIC FILING OF A SEQUENCE LISTING

Pursuant to 37 C.F.R. § 1.821, a Sequence Listing ASCII text file entitled “035-PCT_ST25.txt,” 332 KB in size, generated Jan. 13, 2022, has been submitted via EFS-Web is provided in lieu of a paper copy. This Sequence Listing is hereby incorporated by reference into the specification for its disclosures.

### TECHNICAL FIELD

This disclosure is in the technical field of synthetic biology and metabolic engineering. More particularly, this disclosure is in the technical field of cultivation of metabolically engineered host cells. This disclosure describes a method of producing an oligosaccharide comprising a lacto-N-triose (LN3; GlcNAc-beta1,3-Gal-beta1,4-Glc) as a core trisaccharide by cultivation with a genetically modified cell, as well as the genetically modified cell used in the method. The genetically modified cell comprises at least one nucleic acid sequence coding for a galactoside beta-1,3-N-acetylglucosaminyltransferase and a glycosyltransferase involved in the synthesis of an oligosaccharide comprising LN3 as a core trisaccharide and at least one nucleic acid sequence expressing a membrane protein. Furthermore, this disclosure provides for a purification of the oligosaccharide comprising LN3 as a core trisaccharide from the cultivation.

### BACKGROUND

Today, more than 80 compounds belonging to the family of Human Milk Oligosaccharides (HMOs) have been structurally characterized. These HMOs represent a class of complex oligosaccharides that function as prebiotics. Additionally, the structural homology of HMO to epithelial epitopes accounts for protective properties against bacterial pathogens. Within the infant gastrointestinal tract, HMOs selectively nourish the growth of selected bacterial strains and are, thus, priming the development of a unique gut microbiota in breast milk-fed infants. Some of these HMOs require the presence of particular oligosaccharide structures having a core structure of GlcNAc-beta1,3-Gal-beta1,4-Glc (lacto-N-triose, LN3) that most likely exhibit a particular biological activity. Production of these oligosaccharides requires the action of a galactoside beta-1,3-N-acetylglucosaminyltransferase that transfers an N-acetylglucosamine (GlcNAc) residue from a UDP-GlcNAc donor to a lactose acceptor thereby synthesizing LN3 and further actions of other glycosyltransferases that further modify the LN3 core trisaccharide. In microbial fermentative production, oligosaccharides with LN3 as a core trisaccharide are in many cases produced intracellularly in the industrial production host. One problem identified in the art as the true difficulty in producing oligosaccharides in cells is the intracellular enrichment of the produced oligosaccharides and their extraction. The intracellular enrichment is deemed to be responsible for the product-inhibitory effect on the production of the desired oligosaccharide. Synthesis may become slow or the desired oligosaccharide may reach cytotoxic concentrations resulting in metabolic arrest or even cell lysis.

### BRIEF SUMMARY

Provided are tools and methods by means of which an oligosaccharide with LN3 as a core trisaccharide can be produced in an efficient, time and cost-effective way and that yields high amounts of the desired product.

Provided are method and a cell for the production of an oligosaccharide with LN3 as a core trisaccharide wherein the cell is genetically modified for the production of an oligosaccharide with LN3 as a core trisaccharide and comprises at least one nucleic acid sequence encoding an enzyme involved in synthesis of an oligosaccharide with LN3 as a core trisaccharide, more specifically the cell comprises a nucleic acid sequence coding for a galactoside beta-1,3-N-acetylglucosaminyltransferase thereby synthesizing LN3 and at least one other glycosyltransferase thereby synthesizing an oligosaccharide with LN3 as a core trisaccharide. The cell furthermore also expresses a membrane protein, more specifically the cell furthermore also expresses a membrane protein previously unknown to improve production and/or enable and/or enhance efflux of an oligosaccharide with LN3 as a core trisaccharide according to this disclosure.

Surprisingly it has now been found that the membrane proteins used in this disclosure provide for newly identified membrane proteins, more specifically this disclosure provide for newly identified membrane proteins previously unknown to enable transport of an oligosaccharide with LN3 as a core trisaccharide and having a positive effect on fermentative production of the oligosaccharide with LN3 as a core trisaccharide, providing a better yield, productivity, specific productivity and/or growth speed when used to genetically engineer a host cell producing the of oligosaccharide with LN3 as a core trisaccharide.

The disclosure also provides methods for producing an oligosaccharide with LN3 as a core trisaccharide. The oligosaccharide with LN3 as a core trisaccharide is obtained with a host cell comprising the membrane protein of this disclosure.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of aspects of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

In this document and in its claims, the verbs “to comprise,” “to have” and “to contain,” and their conjugations are used in their non-limiting sense to mean that items following the word are included, but items not specifically mentioned are not excluded. The verb “to consist essentially of” means that additional component(s) may be present than the ones specifically identified, the additional component(s) not altering the unique characteristic of the disclosure. Throughout the disclosure and claims, unless specifically stated otherwise, the verbs “to comprise,” “to have” and “to contain,” and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the element is present, unless the context clearly requires that there is one and only one of the elements. The indefinite article “a” or “an” thus usually means “at least one.”

According to this disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to this disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The term “endogenous,” within the context of this disclosure refers to any polynucleotide, polypeptide or protein sequence that is a natural part of a cell and is occurring at its natural location in the cell chromosome.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of the gene in any phase of the production process of the oligosaccharide with LN3 as a core trisaccharide. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression is obtained by means of common well-known technologies for a skilled person (such as the usage of siRNA, CrispR, CrispRi, recombineering, homologous recombination, ssDNA mutagenesis, RNAi, miRNA, asRNA, mutating genes, knocking-out genes, transposon mutagenesis, . . . ) that are used to change the genes in such a way that they are less-able (i.e., statistically significantly ‘less-able’ compared to a functional wild-type gene) or completely unable (such as knocked-out genes) to produce functional final products. Overexpression or expression is obtained by means of common well-known technologies for a skilled person, wherein the gene is part of an “expression cassette” that relates to any sequence in which a promoter sequence, untranslated region sequence (containing either a ribosome binding sequence or Kozak sequence), a coding sequence (for instance, a membrane protein gene sequence) and optionally a transcription terminator is present, and leading to the expression of a functional active protein. The expression is either constitutive or conditional or regulated or tuneable.

The term “constitutive expression” is defined as expression that is not regulated by transcription factors other than the subunits of RNA polymerase (e.g., the bacterial sigma factors) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. RNA polymerase binds a specific sequence to initiate transcription, for instance, via a sigma factor in prokaryotic hosts.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wild type” refers to the commonly known genetic or phenotypical situation as it occurs in nature.

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, this disclosure contemplates making functional variants by modifying the structure of a membrane protein as used in this disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, as in case of this disclosure to provide better yield, productivity, and/or growth speed than a cell without the variant.

The term “functional homolog” as used herein describes those molecules that have sequence similarity and also share at least one functional characteristic such as a biochemical activity. In the context of this disclosure, a functional homolog of a membrane protein ‘Z’ according to the disclosure (usually indicated with a SEQ ID NO) refers to a membrane protein that is able to transport an oligosaccharide with LN3 as a core saccharide as described herein, i.e., the functional homolog retains the functional characteristic of membrane protein ‘Z’ to transport an oligosaccharide with LN3 as a core saccharide. More specifically, the term “functional homolog” as used herein describes those proteins that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment,” with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription, or translation. A “polynucleotide fragment” refers to any subsequence of a polynucleotide, typically, of at least about 9 consecutive nucleotides, for example, at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide.

Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide that performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 20 amino acid residues in length, for example, at least about 30 amino acid residues in length. Preferentially a fragment is a functional fragment that has at least one property or activity of the polypeptide from which it is derived, such as, for example, the fragment can include a functional domain or conserved domain of a polypeptide. Hence, in the context of this disclosure, a functional fragment of a membrane protein ‘Z’ according to the disclosure (usually indicated with a SEQ ID NO) hence refers to a fragment that retains the functional characteristic of membrane protein ‘Z’ to transport an oligosaccharide with LN3 as a core saccharide. A domain can be characterized, for example, by a Pfam (El-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (www.ncbi.nlm.nih.gov/cdd) (Lu et al., Nucleic Acids Res. 48 (2020) D265-D268), or a PTHR domain (www.pantherdb.org) (Mi et al., Nucleic Acids. Res. 41 (2013) D377-D386; Thomas et al., Genome Research 13 (2003) 2129-2141). It should be understood for those skilled in the art that for the databases used herein, comprising Pfam 32.0 (released September 2018), CDD v3.17 (released 3 Apr. 2019), eggnogdb 4.5.1 (released September 2016), InterPro 75.0 (released 4 Jul. 2019) and TCDB (released 17 Jun. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

As such, a fragment of a polypeptide SEQ ID NO preferably means a polypeptide sequence that comprises or consists of an amount of consecutive amino acid residues from the polypeptide SEQ ID NO and wherein the amount of consecutive amino acid residues is preferably at least 50.0%, 60.0%, 70.0%, 80.0%, 81.0%, 82.0%, 83.0%, 84.0%, 85.00% 86.00% 87.00% 88.00% 89.00% 90.00% 91.00% 92.0%, 93.0%, 94.00% 95.00% 95.5% 96.0% 96.5%, 97.0% 97.5%, 98.0% 98.5%, 99.0% 99.5%, 100% preferably at least 80.0%, more preferably at least 87.0%, even more preferably at least 90.0%, even more preferably at least 95.0%, most preferably at least 97.0% of the full-length of the polypeptide SEQ ID NO and that performs at least one biological function of the intact polypeptide in substantially the same manner, preferably to a similar or greater extent, as does the intact polypeptide that can be routinely assessed by the skilled person. In the context of this disclosure, a functional fragment of a membrane protein ‘Z’ according to the disclosure (usually indicated with a SEQ ID NO) hence refers to a fragment that retains the functional characteristic of membrane protein ‘Z’ to transport an oligosaccharide with LN3 as a core saccharide. As such, a fragment of a polypeptide SEQ ID NO preferably means a polypeptide sequence that comprises or consists of the polypeptide SEQ ID NO, wherein an amount of consecutive amino acid residues is missing and wherein the amount is no more than 50.0%, 40.0%, 30.0% of the full-length of the polypeptide SEQ ID NO, preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polypeptide SEQ ID NO and that performs at least one biological function of the intact polypeptide in substantially the same manner, preferably to a similar or greater extent, as does the intact polypeptide that can be routinely assessed by the skilled person. The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle (galaxy-iuc.github/io/emboss-5.0-docs/needle.html).

The BLAST (Basic Local Alignment Search Tool) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: blast.ncbi.nlm.nib.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HIMM) iterations: default (0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (galaxy-iuc.github/io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having an amino acid sequence having at least 80% overall sequence identity (or a protein sequence having at least 80% overall sequence identity) to the full-length sequence of a reference polypeptide sequence is to be understood as that the sequence has 80%, 81%, 82%, 83%, 84%, 85%, 86%, 87%, 88%, 89%, 900%, 91%, 92%, 93%, 94%, 95%, 96%, 97%, 98%, 99%, 100% overall sequence identity to the full-length of the amino acid sequence of the reference polypeptide sequence. Throughout the disclosure, unless explicitly specified otherwise, a polypeptide comprising/consisting/having/represented by an amino acid sequence having at least 80% sequence overall sequence identity to the full-length amino acid sequence of a reference polypeptide, usually indicated with a SEQ ID NO, preferably has at least 85%, 90%, 91%, 92.00%, 93.00%, 94.00%, 95.00%, 96.00%, 97.00%, 98.00% or 99.00%, more preferably has at least 85%, even more preferably has at least 90%, even more preferably has at least 95.00%, even more preferably has at least 97.00%, most preferably at least 99.00%, overall sequence identity to the full length reference sequence. In the context of this disclosure, a polypeptide having an amino acid sequence having e.g., at least 80% overall sequence identity (or a protein sequence having e.g., at least 80% overall sequence identity) to the full-length sequence of a reference membrane protein ‘Z’ (usually indicated with a SEQ ID NO) refers to a polypeptide (i.e., membrane protein) that is able to transport an oligosaccharide with LN3 as a core saccharide as described herein, i.e., the polypeptide retains the functional characteristic of the reference membrane protein ‘Z’ to transport an oligosaccharide with LN3 as a core saccharide.

For the purpose of this disclosure, the overall sequence identity of a polypeptide is preferably determined by the program EMBOSS Needle 5.0 (galaxy-iuc.github/io/emboss-5.0-docs/needle.html), preferably with default parameters (the substitution matrix EBLOSUM62, the gap opening penalty 10, and the gap extension penalty 0.5) and preferably with sequences of mature proteins (i.e., without taking into account secretion signals or transit peptides).

The term “glycosyltransferase” as used herein refers to an enzyme capable to catalyze the transfer of sugar moieties from activated donor molecules to specific acceptor molecules, forming glycosidic bonds. The as such synthesized oligosaccharides can be of the linear type or of the branched type and can contain multiple monosaccharide building blocks. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (Carbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases and alpha-1,6-fucosyltransferases that catalyze the transfer of a Fuc residue from GDP-Fuc onto a glycan acceptor via alpha-glycosidic bonds. Fucosyltransferases can be found but are not limited to the GT10, GT11, GT23, GT65 and GT68 CAZy families. Sialyltransferases are glycosyltransferases that transfer a sialyl group (like Neu5Ac or Neu5Gc) from a donor (like CMP-Neu5Ac or CMP-Neu5Gc) onto a glycan acceptor. Sialyltransferases comprise alpha-2,3-sialyltransferases and alpha-2,6-sialyltransferases that catalyze the transfer of a sialyl group onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto a glycan acceptor. Galactosyltransferases comprise beta-1,3-galactosyltransferases, beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto a glycan acceptor. N-acetylglucosaminyltransferases can be found but are not limited to GT2 and GT4 CAZy families. N-acetylgalactosaminyltransferases are glycosyltransferases that transfer an N-acetylgalactosamine group (GalNAc) from an UDP-N-acetylgalactosamine (UDP-GalNAc) donor onto a glycan acceptor. N-acetylgalactosaminyltransferases can be found but are not limited to GT7, GT12 and GT27 CAZy families. N-acetylmannosaminyltransferases are glycosyltransferases that transfer an N-acetylmannosamine group (ManNAc) from an UDP-N-acetylmannosamine (UDP-ManNAc) donor onto a glycan acceptor. Xylosyltransferases are glycosyltransferases that transfer a xylose residue (Xyl) from an UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from an UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto a glycan acceptor. UDP-4-amino-4,6-dideoxy-N-acetyl-beta-L-altrosamine transaminases are glycosyltransferases that use an UDP-2-acetamido-2,6-dideoxy-L-arabino-4-hexulose in the biosynthesis of pseudaminic acid, which is a sialic acid-like sugar that is used to modify flagellin. Fucosaminyltransferases are glycosyltransferases that transfer an N-acetylfucosamine residue from a dTDP-N-acetylfucosamine or an UDP-N-acetylfucosamine donor onto a glycan acceptor.

The term “galactoside beta-1,3-N-acetylglucosaminyltransferase” refers to a glycosyltransferase that is capable to transfer an N-acetylglucosamine (GlcNAc) residue from UDP-GlcNAc to the terminal galactose residue of lactose in a beta-1,3 linkage.

The terms “nucleotide-sugar” or “activated sugar” or “nucleoside” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LdmanHep), D-glycero-D-manno-Heptopyranose (DdmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O—[(R)-1-carboxyethyl]-2-deoxy-D-glucopyranose, 2-Acetamido-3-O—[(R)-carboxyethyl]-2-deoxy-D-glucopyranose, 2-Glycolylamido-3-O—[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose, galactose, N-acetylglucosamine, glucosamine, mannose, xylose, N-acetylmannosamine, N-acetylneuraminic acid, N-glycolylneuraminic acid, N-acetylgalactosamine, galactosamine, fucose, rhamnose, glucuronic acid, gluconic acid, fructose and polyols.

“Oligosaccharide” as the term is used herein and as generally understood in the state of the art, refers to a saccharide polymer containing a small number, typically three to twenty, of simple sugars, i.e., monosaccharides. The oligosaccharide as used in this disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. Each monosaccharide can be in the cyclic form (e.g., pyranose or furanose form). An oligosaccharide can contain both alpha- and beta-glycosidic bonds or can contain only beta-glycosidic bonds. The terms “glycan” and “polysaccharide” are used interchangeably and refer to a compound comprising a large number of monosaccharides linked glycosidically. The term glycan is commonly used for those compounds containing more than ten monosaccharide residues.

The term “oligosaccharide with LN3 as a core trisaccharide” as used herein refers to an oligosaccharide being lacto-N-triose or that contains lacto-N-triose that is further glycosylated. Preferably, the oligosaccharide as described herein contains monosaccharides selected from the list as used herein above. Examples of oligosaccharides of this disclosure include but are not limited to Lewis-type antigen oligosaccharides and mammalian milk oligosaccharides (MMOs), preferably human milk oligosaccharides (HMOs), that contain LN3 as a core trisaccharide. Examples comprise lacto-N-triose, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, lacto-N-hexaose (LNH), lacto-N-neohexaose (LNnH), para-lacto-N-hexaose (pLNnH), para-lacto-N-neohexaose (pLNH), difucosyl-lacto-N-hexaose, difucosyl-lacto-N-neohexaose, lacto-N-pentaose (LNP), lacto-N-neopentaose, para lacto-N-pentaose, para lacto-N-neopentaose, lacto-N-novopentaose I, lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, Sialyl-lacto-N-tetraose a, Sialyl-lacto-N-tetraose b, Sialyl-lacto-N-tetraose c, Sialyl-lacto-N-tetraose d. Preferably, an oligosaccharide of the disclosure (i.e., an oligosaccharide with LN3 as a core trisaccharide as defined herein) is a mammalian milk oligosaccharide (MMO), more preferably a human milk oligosaccharide, even more preferably a HMO or MMO having LNT or LNnT as a core tetrasaccharide, even more preferably a MMO having LNT or LNnT as a core tetrasaccharide, most preferably LNT or LNnT. It is also preferred in the context of the disclosure that the oligosaccharide of the disclosure is a neutral oligosaccharide (i.e., the oligosaccharide does not have a negative charge originating from a carboxylic group).

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in this disclosure, are used interchangeably.

The terms “LNT,” “lacto-N-tetraose,” “lacto-N-tetraose” or “Galβ1-3GlcNAcβ1-3Galβ1-4Glc” as used in this disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in this disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in this disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in this disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in this disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc.”

Mammalian milk oligosaccharides comprise oligosaccharides present in milk found in any phase during lactation including colostrum milk from humans and mammals including but not limited to cows (Bos taurus), sheep (Ovis aries), goats (Capra aegagrus hircus), bactrian camels (Camelus bactrianus), horses (Equusferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus).

The term “pathway” as used herein is a biochemical pathway comprising the enzymes and their respective genes involved in the synthesis of an oligosaccharide as defined herein. The pathway for production of an oligosaccharide comprises but is not limited to pathways involved in the synthesis of a nucleotide-activated sugar and the transfer of the nucleotide-activated sugar to an acceptor to create an oligosaccharide of this disclosure. Examples of such pathway comprise but are not limited to a fucosylation, sialylation, galactosylation, N-acetylglucosaminylation, N-acetylgalactosylation, mannosylation, N-acetylmannosinylation pathway.

A ‘fucosylation pathway’ as used herein is a biochemical pathway comprising the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3 α 1,4 or α 1,6 fucosylated oligosaccharides.

A ‘sialylation pathway’ is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α 2,3; α 2,6 α 2,8 sialylated oligosaccharides.

A ‘galactosylation pathway’ as used herein is a biochemical pathway comprising the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, 6 hydroxyl group of a mono-, di-, or oligosaccharide.

An ‘N-acetylglucosaminylation pathway’ as used herein is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, 6 hydroxyl group of a mono-, di- or oligosaccharide.

An ‘N-acetylgalactosylation pathway’ as used herein is a biochemical pathway comprising the enzymes and their respective genes, L-glutamine-D-fructose-6-phosphate aminotransferase, phosphoglucosamine mutase, N-acetylglucosamine 1-phosphate uridylyltransferase, UDP-N-acetylglucosamine 4-epimerase, UDP-galactose 4-epimerase, N-acetylgalactosamine kinase and/or UDP-GalNAc pyrophosphorylase combined with a glycosyltransferase leading to an alpha or beta bound N-acetylgalactosamine on a mono-, di- or oligosaccharide.

A ‘mannosylation pathway’ as used herein is a biochemical pathway comprising the enzymes and their respective genes, mannose-6-phosphate isomerase, phosphomannomutase and/or mannose-1-phosphate guanyltransferase combined with a glycosyltransferase leading to an alpha or beta bound mannose on a mono-, di- or oligosaccharide.

An ‘N-acetylmannosinylation pathway’ as used herein is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to an alpha or beta bound N-acetylmannosamine on a mono-, di- or oligosaccharide.

The term “membrane proteins” as used herein refers to proteins that are part of or interact with the cells membrane and control the flow of molecules and information across the cell. The membrane proteins are thus involved in transport, be it import into or export out of the cell.

Such membrane proteins can be porters, P-P-bond-hydrolysis-driven transporters or β-Barrel Porins as defined by the Transporter Classification Database that is operated and curated by the Saier Lab Bioinformatics Group available via www.tcdb.org and providing a functional and phylogenetic classification of membrane transport proteins This Transporter Classification Database details a comprehensive IUBMB approved classification system for membrane transport proteins known as the Transporter Classification (TC) system. The TCDB classification searches as described here are defined based on TCDB. Org as released on 17 Jun. 2019.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Membrane proteins are included in this class when they utilize a carrier-mediated process to catalyze uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy, of secondary carriers (Forrest et al., Biochim. Biophys. Acta 1807 (2011) 167-188). These systems are usually stereospecific. Solute:solute countertransport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolytes, siderophores.

Membrane proteins are included in the class of P-P-bond hydrolysis-driven transporters if they hydrolyze the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379). The membrane protein may or may not be transiently phosphorylated, but the substrate is not phosphorylated. Substrates that are transported via the class of P-P-bond hydrolysis-driven transporters include but are not limited to cations, heavy metals, beta-glucan, UDP-glucose, lipopolysaccharides, teichoic acid.

The β-Barrel porins membrane proteins form transmembrane pores that usually allow the energy independent passage of solutes across a membrane. The transmembrane portions of these proteins consist exclusively of β-strands that form a β-barrel (Saier et al., Nucleic Acids Res. 44 (2016) D372-D379). These porin-type proteins are found in the outer membranes of Gram-negative bacteria, mitochondria, plastids, and possibly acid-fast Gram-positive bacteria. Solutes that are transported via these β-Barrel porins membrane proteins include but are not limited to nucleosides, raffinose, glucose, beta-glucosides, oligosaccharides.

The term “enabled efflux” means to introduce the activity of transport of a solute over the cytoplasm membrane and/or the cell wall. The transport may be enabled by introducing and/or increasing the expression of a transporter protein as described in this disclosure. The term “enhanced efflux” means to improve the activity of transport of a solute over the cytoplasm membrane and/or the cell wall. The transport may be enhanced by introducing and/or increasing the expression of a transporter protein as described in this disclosure. “Expression” of a transporter protein is defined as “overexpression” of the gene encoding the transporter protein in the case the gene is an endogenous gene or “expression” in the case the gene encoding the transporter protein is a heterologous gene that is not present in the wild type strain.

As used herein, the term “cell productivity index (CPI)” refers to the mass of the product produced by the recombinant cells divided by the mass of the recombinant cells produced in the culture.

The term “non-native” as used herein with reference to an oligosaccharide with LN3 as a core trisaccharide indicates that the oligosaccharide is i) not naturally produced or ii) when naturally produced not in the same amounts by the cell; and that the cell has been genetically modified to be able to produce the oligosaccharide or to have a higher production of the oligosaccharide.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the oligosaccharide with LN3 as a core trisaccharide that is produced by the cell in whole broth, i.e., inside (intracellularly) as well as outside (extracellularly) of the cell.

The term “precursor” as used herein refers to substances that are taken up or synthetized by the cell for the specific production of an oligosaccharide according to this disclosure. In this sense a precursor can be an acceptor as defined herein, but can also be another substance, metabolite, which is first modified within the cell as part of the biochemical synthesis route of the oligosaccharide. Examples of such precursors comprise the acceptors as defined herein, and/or glucose, galactose, fructose, glycerol, sialic acid, fucose, mannose, maltose, sucrose, lactose, glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetyl-glucosamine-6-phosphate, N-acetyl-glucosamine, N-acetyl-mannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid-9-phosphate, CMP-sialic acid, mannose-6-phosphate, mannose-1-phosphate, GDP-mannose, GDP-4-dehydro-6-deoxy-α-D-mannose, and/or GDP-fucose.

Optionally, the cell is transformed to comprise at least one nucleic acid sequence encoding a protein selected from the group comprising lactose transporter, N-acetylneuraminic acid transporter, fucose transporter, transporter for a nucleotide-activated sugar wherein the transporter internalizes a to the medium added precursor for the synthesis of the oligosaccharide of this disclosure.

The term “acceptor” as used herein refers to a mono-, di- or oligosaccharide that can be modified by a glycosyltransferase. Examples of such acceptors comprise glucose, galactose, fructose, glycerol, sialic acid, fucose, mannose, maltose, sucrose, lactose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, and oligosaccharide containing 1 or more N-acetyllactosamine units and/or 1 or more lacto-N-biose units or an intermediate into oligosaccharide, fucosylated and sialylated versions thereof.

### DETAILED DESCRIPTION

In a first aspect, this disclosure provides a metabolically engineered cell for the production of an oligosaccharide comprising LN3 as a core trisaccharide as defined herein. Herein, a metabolically engineered cell is provided that comprises at least one nucleic acid sequence for a galactoside beta-1,3-N-acetylglucosaminyltransferase that transfers an N-acetylglucosamine (GlcNAc) residue from a UDP-GlcNAc donor to a lactose acceptor thereby synthesizing LN3, and that further comprises i) overexpression of an endogenous membrane protein and/or ii) expression of a heterologous membrane protein providing improved production and/or enabled and/or enhanced efflux of an oligosaccharide comprising LN3 as a core trisaccharide. The cell may further comprise at least one nucleic acid sequence coding for a glycosyltransferase that is capable to modify the LN3 to an oligosaccharide comprising LN3 as a core trisaccharide.

According to a second aspect, this disclosure provides a method for the production of an oligosaccharide with LN3 as a core trisaccharide by a genetically modified cell. The method comprises the steps of:
1) providing a cell capable of producing an oligosaccharide with LN3 as a core trisaccharide, the cell comprising at least one nucleic acid sequence for a galactoside beta-1,3-N-acetylglucosaminyltransferase that transfers an N-acetylglucosamine (GlcNAc) residue from a UDP-GlcNAc donor to a lactose acceptor thereby synthesizing LN3, the cell further comprising i) overexpression of an endogenous membrane protein, more specifically an endogenous membrane protein involved in the production and/or efflux of an oligosaccharide with LN3 as a core trisaccharide, even more specifically an endogenous membrane protein enabling and/or enhancing production and/or enabling and/or enhancing efflux of an oligosaccharide with LN3 as a core trisaccharide, and/or ii) an expression of an heterologous membrane protein, more specifically an heterologous membrane protein involved in production and/or efflux of an oligosaccharide with LN3 as a core trisaccharide, even more specifically an heterologous membrane protein enabling and/or enhancing production and/or enabling and/or enhancing efflux of an oligosaccharide with LN3 as a core trisaccharide, and
2) cultivating the cell in a medium under conditions permissive for the production of the desired oligosaccharide with LN3 as a core trisaccharide.

Throughout the disclosure, unless specified otherwise, the verbs “cultivate” (and its conjugations) and “culture” are interchangeably used in the context of this disclosure.

In the context of the disclosure, it is preferred that the endogenous membrane protein and/or the heterologous membrane protein of the disclosure is/are not a lactose permease (for example, encoded by the lacy gene or the lac12 gene), preferably wherein the lactose permease is represented with SEQ ID NO: 52. This preferred embodiment however does not exclude the presence of a lactose permease in the cell of the disclosure. The skilled person understands that the cell according to the disclosure (overexpressing an endogenous membrane protein and/or expressing a heterologous membrane protein) may be additionally genetically modified to import lactose in the cell, by the introduction and/or overexpression of a lactose permease (e.g., SEQ ID NO: 52) as described further herein.

Preferably, the cell further comprises at least one nucleic acid sequence coding for a glycosyltransferase that is capable to modify the LN3.

Preferably, the oligosaccharide with LN3 as a core trisaccharide is separated from the cultivation as explained herein.

In the scope of this disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3.

In a preferred embodiment of the method and/or cell of the disclosure, the metabolically engineered cell is modified with gene expression modules wherein the expression from any one of the expression modules is constitutive or is tuneable. The expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred embodiment of this disclosure, the cell is modified with one or more expression modules. The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. The recombinant gene is involved in the expression of a polypeptide acting in the synthesis of the glycosylated product; or the recombinant gene is linked to other pathways in the host cell that are not involved in the synthesis of the glycosylated product. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell that also expresses a heterologous protein.

According to a preferred embodiment of this disclosure, the expression of each of the expression modules is constitutive or tuneable as defined herein.

According to a preferred embodiment of the method and/or cell of the disclosure, the cell is metabolically engineered to comprise a pathway for the production of an oligosaccharide with LN3 as a core trisaccharide as defined herein. In a further preferred embodiment, the cell comprises a recombinant galactoside beta-1,3-N-acetylglucosaminyltransferase capable of modifying lactose or an intermediate into LN3. In an even further preferred embodiment, the cell comprises another recombinant glycosyltransferase capable of modifying LN3 or a derivative of LN3 into an oligosaccharide with LN3 as a core trisaccharide.

In another preferred embodiment of the method and/or cell, the cell is genetically modified to express the de novo synthesis of UDP-GlcNAc. The UDP-GlcNAc can be provided by an enzyme expressed in the cell or by the metabolism of the cell. Such cell producing an UDP-GlcNAc can express enzymes converting, e.g., GlcNAc, which is to be added to the cell, to UDP-GlcNAc. These enzymes may be an N-acetyl-D-glucosamine kinase, an N-acetylglucosamine-6-phosphate deacetylase, a phosphoglucosamine mutase, and an N-acetylglucosamine-1-phosphate uridyltransferase/glucosamine-1-phosphate acetyltransferase from several species including Homo sapiens, Escherichia coli. Preferably, the cell is modified to produce UDP-GlcNAc. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase.

Additionally, or alternatively, the host cell used herein is optionally genetically modified to import lactose in the cell, by the introduction and/or overexpression of a lactose permease. The lactose permease is encoded by, for example, the lacY gene or the lac12 gene.

According to the method and/or cell of the disclosure, the cell expresses a membrane protein. The membrane protein is either an endogenous protein with a modified expression, preferably the endogenous protein is overexpressed; or the membrane protein is a heterologous protein, which can be heterologously expressed by the cell. The heterologously expressed membrane protein will then be introduced and expressed, preferably overexpressed. In another embodiment, the endogenous protein can have a modified expression in the cell that also expresses a heterologous membrane protein. In another embodiment, modified expression of an endogenous membrane protein comprises modified expression of other proteins that map in the same operon of the endogenous membrane protein and/or share common control sequences for expression. In another embodiment, the membrane protein is expressed together with conterminal proteins that share the same regulon. In another embodiment, when the membrane protein is an inner membrane transporter (complex), the membrane protein is expressed together with one or more outer membrane transporter(s). In an alternative embodiment, when the membrane protein is an outer membrane transporter, the membrane protein is expressed together with one or more inner membrane protein(s). In an alternative embodiment, the membrane protein is expressed with one or more inner membrane proteins and/or one or more outer membrane proteins. According to a further embodiment of the disclosure, the polynucleotide encoding the membrane protein is adapted to the codon usage of the respective cell or expression system.

In a further preferred embodiment of the method and/or cell of the disclosure, the membrane protein is selected from the group of porters, P-P-bond-hydrolysis-driven transporters, and β-Barrel Porins.

In a preferred embodiment of the method and/or cell, when the membrane protein is selected from the group of porter membrane proteins, the membrane protein is selected from the group of TCDB classes 2.A.1.1, 2.A.1.2, 2.A.1.3, 2.A.1.6, 2.A.2.2, 2.A.7.1 and 2.A.66.

In an alternative preferred embodiment of the method and/or cell, when the membrane protein is selected from the group of porter membrane proteins, the membrane protein is selected from the group of eggnog families 05E8G, 05EGZ, 05JHE, 07QF7 07QRN, 07RBJ, 0814C and 08N8A.

In another preferred embodiment of the method and/or cell, when the membrane protein is selected from the group of porter membrane proteins, the membrane protein is selected from the PFAM list PF00893, PF01943, PF05977, PF07690 and PF13347.

In another preferred embodiment of the method and/or cell, when the membrane protein is selected from the group of porter membrane proteins, the membrane protein is selected from the interpro list IPR000390, IPR001411, IPR001927, IPR002797, IPR004638, IPR005829, IPR010290, IPR011701, IPR020846, IPR023721, IPR023722, IPR032896, IPR036259 and IPR039672.

In another preferred embodiment of the method and/or cell, when the membrane protein is selected from the group of porter membrane proteins, the membrane protein is selected from MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, MdfA from Enterobacter kobei with SEQ ID NO: 66, MdfA from Lelliottia sp. WB101 with SEQ ID NO: 67, MdfA from Citrobacter freundii with SEQ ID NO: 68, MdfA from Salmonella enterica subsp. Salamae with SEQ ID NO: 69 or MdfA from Shigella flexneri with SEQ ID NO: 70, or functional homolog or functional fragment of any one of the above porter membrane proteins, or a protein sequence having at least 80% sequence identity to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64, 65, 66, 67, 68, 69 or 70, respectively. Throughout the description and claims, the term “a protein sequence having at least 80% sequence identity” is preferably replaced with the term “a protein having at least 80% sequence identity” or “a polypeptide having at least 80% sequence identity.”

In a more preferred embodiment of the method and/or cell, when the membrane protein is selected from the group of porter membrane proteins, the membrane protein is selected from (i.e., represented by) SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, or functional homolog or functional fragment of any one of the porter membrane proteins, or a protein sequence having at least 80% preferably at least 85%, more preferably at least 90%, even more preferably at least 95.00%, most preferably at least 97.00%, sequence identity to the full-length sequence of the membrane proteins with SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, respectively.

In an even more preferred embodiment of the method and/or cell, when the membrane protein is selected from the group of porter membrane proteins, the membrane protein is selected from (i.e., represented by) SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, or functional homolog or functional fragment of any one of the porter membrane proteins, or a protein sequence having:
at least 80% sequence identity to the full-length sequence of the membrane proteins with SEQ ID NOs: 09, 10, 11, 12 or 13, respectively,
at least 90% sequence identity to the full-length sequence of the membrane proteins with SEQ ID NOs: 01, 02, 04, 14, 53, 54, 55, 59, 61, 62, 63, 64, 65, 66, 67 or 69, respectively,
at least 95.00% sequence identity to the full-length sequence of the membrane proteins with SEQ ID NOs: 05, 06, 56, 57 or 68, respectively, or
at least 99.00% sequence identity to the full-length sequence of the membrane proteins with SEQ ID NOs: 58, 60 or 70, respectively.

In an alternative preferred embodiment of the method and/or cell, when the membrane protein is selected from the group of porter membrane proteins, the membrane protein is selected from (i.e., represented by) SEQ ID NOs: 01, 02, 04, 09, 10, 11, 12, 13, 14, 53, 54, 55, 59, 61, 62, 63, 64, 65, 66, 67, or 69, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64, 65, 66, 67, or 69, respectively. More preferably, the membrane protein is selected from (i.e., represented by) SEQ ID NOs: 01, 02, 04, 09, 10, 11, 12, 13, 14, 53, 54, 55, 59, 61, 62, 63, 64, 65, 66, 67, or 69, or functional homolog or functional fragment of any one of the porter membrane proteins, or a protein sequence having at least 90%, preferably at least 95.00%, more preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64, 65, 66, 67, or 69, respectively.

In another preferred embodiment of the method and/or cell, when the membrane protein is selected from the group of porter membrane proteins, the membrane protein is selected from MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64 or MdfA from Kluyvera ascorbata ATCC 33433 with SEQ ID NO: 65, or functional homolog or functional fragment of any one of the above porter membrane proteins, or a protein sequence having at least 80% sequence identity to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64 or 65, respectively.

In a more preferred embodiment of the method and/or cell, when the membrane protein is selected from the group of porter membrane proteins, the membrane protein is selected from (i.e., represented by) SEQ ID NOs: 01, 02, 04, 05, 06, 09, 10, 11, 12, 13, 14, 53, 54, 55, 56, 57, 58, 59, 60, 61, 62, 63, 64 or 65, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 05, 06, 09, 10, 11, 12, 13, 14, 53, 54, 55, 56, 57, 58, 59, 60, 61, 62, 63, 64 or 65, respectively.

In an even more preferred embodiment of the method and/or cell, when the membrane protein is selected from the group of porter membrane proteins, the membrane protein is selected from (i.e., represented by) SEQ ID NOs: 01, 02, 04, 05, 06, 09, 10, 11, 12, 13, 14, 53, 54, 55, 56, 57, 58, 59, 60, 61, 62, 63, 64 or 65, or functional homolog or functional fragment of any one of the porter membrane proteins, or a protein sequence having:
at least 80% sequence identity to the full-length sequence of the membrane proteins with SEQ ID NOs: 09, 10, 11, 12 or 13, respectively,
at least 90% sequence identity to the full-length sequence of the membrane proteins with SEQ ID NOs: 01, 02, 04, 14, 53, 54, 55, 59, 61, 62, 63, 64 or 65, respectively,
at least 95.00% sequence identity to the full-length sequence of the membrane proteins with SEQ ID NOs: 05, 06, 56 or 57, respectively, or
at least 99.00% sequence identity to the full-length sequence of the membrane proteins with SEQ ID NOs: 58 or 60, respectively.

In an alternative preferred embodiment of the method and/or cell, when the membrane protein is selected from the group of porter membrane proteins, the membrane protein is selected from (i.e., represented by) SEQ ID NOs: 01, 02, 04, 09, 10, 11, 12, 13, 14, 53, 54, 55, 59, 61, 62, 63, 64 or 65, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64 or 65, respectively. More preferably, the membrane protein is selected from (i.e., represented by) SEQ ID NOs: 01, 02, 04, 09, 10, 11, 12, 13, 14, 53, 54, 55, 59, 61, 62, 63, 64 or 65, or functional homolog or functional fragment of any one of the porter membrane proteins, or a protein sequence having at least 90%, preferably at least 95.00%, more preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64 or 65, respectively.

The amino acid sequence of such porter membrane protein can be a sequence chosen from SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69 or 70, preferably chosen from SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, more preferably chosen from SEQ ID NOs: 01, 02, 04, 05, 06, 09, 10, 11, 12, 13, 14, 53, 54, 55, 56, 57, 58, 59, 60, 61, 62, 63, 64 or 65, even more preferably chosen from 01, 02, 04, 05, 06, 55, 59, 66 or 68, most preferably chosen from SEQ ID NOs: 01, 02, 04, 05, 06, 55 or 59, of the attached sequence listing, or an amino acid sequence that has least 80% sequence identity, 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, preferably at least 85%, more preferably at least 90%, even more preferably at least 95.00%, even more preferably at least 97.00%, most preferably at least 99.00%, sequence identity to the full-length amino acid sequence of any one of SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69 or 70, respectively.

Alternatively, the amino acid sequence of such porter membrane protein can be a sequence chosen from SEQ ID NOs: 01, 02, 04, 09, 10, 11, 12, 13, 14, 53, 54, 55, 59, 61, 62, 63, 64, 65, 66, 67 or 69, more preferably chosen from SEQ ID NOs: 01, 02, 04, 09, 10, 11, 12, 13, 14, 53, 54, 55, 59, 61, 62, 63, 64 or 65, even more preferably chosen from 01, 02, 04, 55, 59, 66 or 68, even more preferably chosen from SEQ ID NOs: 01, 02, 04, 55 or 59, most preferably chosen from SEQ ID NOs: 01, 02 or 04, even more preferably chosen from SEQ ID NOs: 01 or 02, most preferably SEQ ID NO: 01, of the attached sequence listing, or an amino acid sequence that has least 80% sequence identity, 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, preferably at least 85%, more preferably at least 90%, even more preferably at least 95.00%, even more preferably at least 97.00%, most preferably at least 99.00%, sequence identity to the full-length amino acid sequence of any one of SEQ ID NOs: 01, 02, 04, 09, 10, 11, 12, 13, 14, 53, 54, 55, 59, 61, 62, 63, 64, 65, 66, 67 or 69, respectively.

Exemplary and preferred membrane proteins having a protein sequence having at least 80% sequence identity to the full-length sequence of any one of the membrane proteins with SEQ ID NOs: 01, 02, 04, 05, 06, 53, 54, 55, 56, 57, 58, 59, 60, 61, 62, 63, 64, 65, 66, 67, 68, 69 or 70 are provided in Table 1.

TABLE 1

| Porter | Exemplary membrane protein having |
|---|---|
| membrane | ≥80% sequence identity to full-length |
| protein | of the porter membrane protein |
| SEQ ID NO: 01 | SEQ ID NOs: 71, 72, 73, 74, 75, 76, 77, |
| | 78, 79, 80, 81, 83, 84, 85, 86, 87, 88, 89, |
| | 90, 91, 92, 93, 94, 95, 96, 97, 98 |
| SEQ ID NOs: 02, 04, 05, | SEQ ID NOs: 71, 72, 73, 74, 75, 76, 77, |
| 06, 54, 56, 57, 58, 60, 61, | 78, 79, 80, 81, 82, 83, 84, 85, 86, 87, 88, |
| 62, 66, 67, 68, 69, 70 | 89, 90, 91, 92, 93, 94, 95, 96, 97, 98 |
| SEQ ID NO: 53 | SEQ ID NOs: 71, 72, 73, 74, 75, 77, 78, |
| | 80, 81, 83, 85, 86, 87, 88, 89, 90, 91, 92, |
| | 93, 94, 95, 96, 97, 98 |
| SEQ ID NOs: 55, 59 | SEQ ID NOs: 71, 72, 73, 74, 75, 77, 78, |
| | 79, 80, 81, 82, 83, 84, 85, 86, 87, 88, 89, |
| | 90, 91, 92, 93, 94, 95, 96, 97, 98 |
| SEQ ID NO: 63 | SEQ ID NOs: 71, 72, 73, 74, 75, 76, 77, |
| | 78, 79, 80, 81, 83, 85, 86, 87, 88, 89, 90, |
| | 91, 92, 93, 94, 95, 96, 97, 98 |
| SEQ ID NO: 64 | SEQ ID NOs: 71, 72, 73, 74, 75, 76, 77, |
| | 78, 79, 80, 81, 85, 86, 87, 88, 89, 90, 91, |
| | 92, 93, 94, 95, 96, 97, 98 |
| SEQ ID NO: 65 | SEQ ID NOs: 71, 73, 74, 75, 76, 77, 78, |
| | 79, 80, 81, 83, 85, 86, 87, 88, 89, 90, 91, |
| | 92, 93, 94, 95, 96, 97, 98 |

In a preferred embodiment of the method and/or cell, when the membrane protein is selected from the group of P-P-bond-hydrolysis-driven transporters, the membrane protein is selected from the group of TCDB classes 3.A.1.1 and 3.A.1.2.

In another preferred embodiment of the method and/or cell, when the membrane protein is selected from the group of P-P-bond-hydrolysis-driven transporters, the membrane protein is selected from the group of eggnog families 05CJ1, 05DFW, 05EZD, 05I1K, 07HR3 and 08IJ9.

In another preferred embodiment of the method and/or cell, when the membrane protein is selected from the group of P-P-bond-hydrolysis-driven transporters, the membrane protein is selected from the PFAM list PF00005, PF00528, PF13407 and PF17912.

In another preferred embodiment of the method and/or cell, when the membrane protein is selected from the group of P-P-bond-hydrolysis-driven transporters, the membrane protein is selected from the interpro list IPR000515, IPR003439, IPR003593, IPR005978, IPR008995, IPR013456, IPR015851, IPR017871, IPR025997, IPR027417, IPR028082, IPR035906, and IPR040582.

In another preferred embodiment of the method and/or cell, when the membrane protein is selected from the group of P-P-bond-hydrolysis-driven transporters, the membrane protein is selected from Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20, or functional homolog or functional fragment of any one of the above P-P-bond-hydrolysis-driven transporter membrane proteins or a protein sequence having at least 80% sequence identity to the full-length sequence of any one of the membrane proteins with SEQ ID NOs: 15, 16, 17, 18, 19 or 20, respectively.

In another preferred embodiment of the method and/or cell, when the membrane protein is selected from the group of β-Barrel Porins, the membrane protein is selected from TCDB class 1.B.18.

In another preferred embodiment of the method and/or cell, when the membrane protein is selected from the group of β-Barrel Porins, the membrane protein is selected from the eggnog family 05DAY.

In another preferred embodiment of the method and/or cell, when the membrane protein is selected from the group of β-Barrel Porins, the membrane protein is selected from the PFAM list PF02563, PF10531 and PF18412.

In another preferred embodiment of the method and/or cell, when the membrane protein is selected from the group of β-Barrel Porins, the membrane protein is selected from the interpro list IPR003715, IPR019554 and IPR040716.

In another preferred embodiment of the method and/or cell, when the membrane protein is selected from the group of β-Barrel Porins, the membrane protein is Wza from Escherichia coli K12 MG1655 with SEQ ID NO: 21, or functional homolog or functional fragment thereof or a protein sequence having at least 80% sequence identity to the full-length sequence of the membrane protein with SEQ ID NO: 21.

In an alternative preferred embodiment of the method and/or cell, the membrane protein is selected from the group of TCDB classes 1.B.18, 2.A.1.1, 2.A.1.2, 2.A.1.3, 2.A.1.6, 2.A.2.2, 2.A.7.1, 2.A.66, 3.A.1.1 and 3.A.1.2; the group of eggnog families 05CJ1, 05DAY, 05DFW, 05E8G, 05EGZ, 05EZD, 05I1K, 05JHE, 07HR3, 07QF7 07QRN, 07RBJ, 0814C, 08IJ9 and 08N8A; the PFAM list PF00005, PF00528, PF00893, PF01943, PF02563, PF05977, PF07690, PF10531, PF13347, PF13407, PF17912 and PF18412; the interpro list IPR000390, IPR000515, IPR001411, IPR001927, IPR002797, IPR003439, IPR003593, IPR003715, IPR004638, IPR005829, IPR005978, IPR008995, IPR010290, IPR011701, IPR013456, IPR015851, IPR017871, IPR019554, IPR020846, IPR023721, IPR023722, IPR025997, IPR027417, IPR028082, IPR032896, IPR035906, IPR036259, IPR039672, IPR040582 and IPR040716; MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, MdfA from Enterobacter kobei with SEQ ID NO: 66, MdfA from Lelliottia sp. WB101 with SEQ ID NO: 67, MdfA from Citrobacter freundii with SEQ ID NO: 68, MdfA from Salmonella enterica subsp. Salamae with SEQ ID NO: 69, MdfA from Shigella flexneri with SEQ ID NO: 70, Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or Wza from Escherichia coli K12 MG1655 with SEQ ID NO: 21; or functional homolog or functional fragment of any one of the above membrane proteins; or a protein sequence having at least 80% sequence identity to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64, 65, 66, 67, 68, 69, 70, 15, 16, 17, 18, 19, 20 or 21, respectively.

In an alternative preferred embodiment of the method and/or cell, the membrane protein is selected from the group of TCDB classes 1.B.18, 2.A.1.1, 2.A.1.2, 2.A.1.3, 2.A.1.6, 2.A.2.2, 2.A.7.1, 2.A.66, 3.A.1.1 and 3.A.1.2; the group of eggnog families 05CJ1, 05DAY, 05DFW, 05E8G, 05EGZ, 05EZD, 05I1K, 05JHE, 07HR3, 07QF7 07QRN, 07RBJ, 0814C, 08IJ9 and 08N8A; the PFAM list PF00005, PF00528, PF00893, PF01943, PF02563, PF05977, PF07690, PF10531, PF13347, PF13407, PF17912 and PF18412; the interpro list IPR000390, IPR000515, IPR001411, IPR001927, IPR002797, IPR003439, IPR003593, IPR003715, IPR004638, IPR005829, IPR005978, IPR008995, IPR010290, IPR011701, IPR013456, IPR015851, IPR017871, IPR019554, IPR020846, IPR023721, IPR023722, IPR025997, IPR027417, IPR028082, IPR032896, IPR035906, IPR036259, IPR039672, IPR040582 and IPR040716; MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or Wza from Escherichia coli K12 MG1655 with SEQ ID NO: 21; or functional homolog or functional fragment of any one of the above membrane proteins; or a protein sequence having at least 80% sequence identity, preferably at least 90% sequence identity, to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64, 65, 15, 16, 17, 18, 19, 20 or 21, respectively.

The TCDB classes are classified as defined on TCDB.org as released on 17 Jun. 2019. The eggnog families are classified as defined on eggnogdb 4.5.1 as released on September 2016. The PFAM list is classified as defined on Pfam 32.0 as released on September 2018. The interpro lists are as defined by InterPro 75.0 as released on 4 Jul. 2019.

As used herein, a protein having an amino acid sequence having at least 80% sequence identity to the full-length sequence of any of the enlisted membrane proteins, is to be understood as that the sequence has 80%, 81%, 82%, 83%, 84%, 85%, 86%, 87%, 88%, 89%, 90%, 91%, 91.50% 92.00% 92.50% 93.00% 93.50% 94.00% 94.50% 95.000% 95.50%, 96.00%, 96.50%, 97.00%, 97.50%, 98.00%, 98.50%, 99.00%, 99.50%, 99.60%, 99.70%, 99.80%, 99.90% sequence identity to the full length of the amino acid sequence of the respective membrane protein. In the context of this disclosure, a protein/polypeptide having an amino acid sequence (e.g., a membrane protein having a protein sequence as disclosed throughout the description and claims) having, for example, at least 80% sequence identity to the full-length sequence of a reference membrane protein ‘Z’ (usually indicated with a SEQ ID NO) refers to a protein (i.e., membrane protein) that is able to transport an oligosaccharide with LN3 as a core saccharide as described herein, i.e., the protein retains the functional characteristic of the reference membrane protein ‘Z’ to transport an oligosaccharide with LN3 as a core saccharide. Likewise, a protein sequence having, for example, at least 80% sequence identity to the full-length sequence of a reference membrane protein ‘Z’ (usually indicated with a SEQ ID NO) refers to a protein (i.e., membrane protein) that is able to transport an oligosaccharide with LN3 as a core saccharide as described herein, i.e., the protein retains the functional characteristic of the reference membrane protein ‘Z’ to transport an oligosaccharide with LN3 as a core saccharide. The ability of a membrane protein to transport an oligosaccharide with LN3 as a core saccharide as described herein can be assessed by the skilled person, for example, as described in the present Examples.

The amino acid sequence of such membrane protein can be a sequence chosen from SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69 or 70, preferably chosen from SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64 or 65, more preferably chosen from SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, even more preferably chosen from SEQ ID NOs: 01, 02, 04, 05, 06, 09, 10, 11, 12, 13, 14, 53, 54, 55, 56, 57, 58, 59, 60, 61, 62, 63, 64 or 65, even more preferably chosen from 01, 02, 04, 05, 06, 55, 59, 66 or 68, most preferably chosen from SEQ ID NOs: 01, 02, 04, 05, 06, 55 or 59, of the attached sequence listing, or an amino acid sequence that has least 80% sequence identity, 80%, 81%, 82%, 83%, 84%, 85%, 86%, 87%, 88%, 89%, 90%, 91%, 91.500% 92.00% 92.50% 93.00% 93.50% 94.00% 94.50% 95.00% 95.50% 96.00% 96.500% 97.00% 97.50% 98.00% 98.50% 99.00% 99.50% 99.60% 99.70% 99.80% 99,900% preferably at least 85%, more preferably at least 90%, even more preferably at least 95.00%, even more preferably at least 97.00%, most preferably at least 99.00%, sequence identity to the full-length amino acid sequence of any one of SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69 or 70, respectively.

Alternatively, the amino acid sequence of such membrane protein can be a sequence chosen from SEQ ID NOs: 01, 02, 04, 09, 10, 11, 12, 13, 14, 15, 16, 17, 18, 19, 20, 21, 53, 54, 55, 59, 61, 62, 63, 64, 65, 66, 67 or 69, preferably chosen from SEQ ID NOs: 01, 02, 04, 09, 10, 11, 12, 13, 14, 53, 54, 55, 59, 61, 62, 63, 64, 65, 66, 67 or 69, more preferably chosen from SEQ ID NO: 01, 02, 04, 09, 10, 11, 12, 13, 14, 53, 54, 55, 59, 61, 62, 63, 64 or 65, even more preferably chosen from 01, 02, 04, 55, 59, 66 or 68, even more preferably chosen from SEQ ID NOs: 01, 02, 04, 55 or 59, even more preferably chosen from SEQ ID NOs: 01, 02 or 04, even more preferably chosen from SEQ ID NOs: 01 or 02, most preferably SEQ ID NO: 01, of the attached sequence listing, or an amino acid sequence that has least 80% sequence identity, 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, preferably at least 85%, more preferably at least 90%, even more preferably at least 95.00%, even more preferably at least 97.00%, most preferably at least 99.00%, sequence identity to the full-length amino acid sequence of any one of SEQ ID NOs: 01, 02, 04, 09, 10, 11, 12, 13, 14, 15, 16, 17, 18, 19, 20, 21, 53, 54, 55, 59, 61, 62, 63, 64, 65, 66, 67 or 69, respectively.

In a further embodiment of the method and/or cell of this disclosure, the host cell expresses a membrane protein that is a transporter protein involved in transport of compounds across the outer membrane of the cell wall. Preferably, the cell is transformed to comprise at least one nucleic acid sequence encoding a protein selected from the group comprising a lactose transporter, a glucose transporter, a galactose transporter or a transporter for a nucleotide-activated sugar like, for example, a transporter for UDP-GlcNAc.

According to another preferred embodiment of the method and/or cell of the disclosure, the cell expresses more than one membrane protein.

In a more preferred alternative embodiment, when the membrane protein is Blon_0247 from B. longum subsp. Infantis (strain ATCC 15697) with SEQ ID NO: 10, the Blon_0247 is expressed together with Blon_0245 from B. longum subsp. Infantis (strain ATCC 15697).

In a more preferred alternative embodiment, when the membrane protein is Blon2331 from B. longum subsp. Infantis (strain ATCC 15697) with SEQ ID NO: 9, the Blon2331 is expressed together with Blon2332.

In a more preferred alternative embodiment, when the membrane protein is Bjnodi from Bradyrhizobium japonicum USDA 110 with SEQ ID NO: 15, the Bjnodi is expressed together with nodulation factor nodj.

In a more preferred alternative embodiment, when the membrane protein is wza from E. coli K-12 MG1655 with SEQ ID NO: 20, the wza is expressed together with any one or more of wzx, wzb and/or wzc.

According to another preferred embodiment of the method and/or cell of the disclosure, the cell expresses a glycosyltransferase selected from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a preferred embodiment of the method and/or cell of the disclosure, the cell is modified in the expression or activity of at least one of the glycosyltransferases. In a preferred embodiment, the glycosyltransferase is an endogenous protein of the cell with a modified expression or activity, preferably the endogenous glycosyltransferase is overexpressed; alternatively the glycosyltransferase is a heterologous protein that is heterogeneously introduced and expressed in the cell, preferably overexpressed. The endogenous glycosyltransferase can have a modified expression in the cell that also expresses a heterologous glycosyltransferase.

In a further preferred embodiment of the method and/or cell of the disclosure, the cell expresses an N-acetylglucosamine beta-1,3-galactosyltransferase that transfers a galactose (Gal) from a UDP-Gal donor to the terminal GlcNAc residue of LN3 in a beta-1,3 linkage thereby producing lacto-N-tetraose (LNT; Gal-beta1,3-GlcNAc-beta1,3-Gal-beta1,4-Glc).

In a further preferred embodiment of the method and/or cell of the disclosure, the cell produces 90 g/L or more of LNT in the whole broth and/or in the supernatant and/or wherein the LNT in the whole broth and/or in the supernatant has a purity of at least 80% measured on the total amount of LNT and LN3 produced by the cell in the whole broth and/or supernatant, respectively. Preferably, the cell produces 90 g/L or more of LNT in the supernatant wherein the LNT has a purity of at least 80% measured on the total amount of LNT and LN3 produced by the cell in the supernatant. In a more preferred embodiment of the method and/or cell of the disclosure, the 90 g/L or more of LNT in the whole broth and/or supernatant is obtained with the cell cultivated in a cultivation process, preferably a fermentation process. In another more preferred embodiment of the method and/or cell of the disclosure, the purity of LNT in the whole broth and/or supernatant of at least 80% measured on the total amount of LNT and LN3 produced by the cell in the whole broth and/or supernatant is obtained with the cell cultivated in a cultivation process, preferably a fermentation process.

A purity of LNT of at least 80 percent on the total amount of LNT and LN3 in the whole broth or in the supernatant should be understood as the amount of the LNT in the mixture of LNT and LN3 in the whole broth or in the supernatant, respectively, of 80% or more, comprising 80, 81, 82, 83, 84, 85, 86, 87, 88, 89, 90, 91, 92, 93, 94, 95, 95.5, 96, 96.5, 97, 97.5, 98, 98.5, 99 or 99.5% of LNT measured on the total amount of LNT and LN3 produced by the cell in the whole broth or in the supernatant, respectively.

In an additional and/or alternative further preferred embodiment of the method and/or cell of the disclosure, the cell expresses an N-acetylglucosamine beta-1,4-galactosyltransferase that transfers a galactose (Gal) from a UDP-Gal donor to the terminal GlcNAc residue of LN3 in a beta-1,4 linkage, thereby producing lacto-N-neotetraose (LNnT; Gal-beta1,4-GlcNAc-beta1,3-Gal-beta1,4-Glc).

In a further preferred embodiment of the method and/or cell of the disclosure, the cell produces 70 g/L or more, preferably 90 g/L or more, of LNnT in the whole broth and/or in the supernatant and/or wherein the LNnT in the whole broth and/or in the supernatant has a purity of at least 80% measured on the total amount of LNnT and LN3 produced by the cell in the whole broth and/or in the supernatant, respectively. Preferably, the cell produces 70 g/L or more, preferably 90 g/L or more, of LNnT in the supernatant wherein the LNnT has a purity of at least 80% measured on the total amount of LNnT and LN3 produced by the cell in the supernatant. In a more preferred embodiment of the method and/or cell of the disclosure, the 70 g/L or more, preferably 90 g/L or more, of LNnT in the whole broth and/or supernatant is obtained with the cell cultivated in a cultivation process, preferably a fermentation process. In another more preferred embodiment of the method and/or cell of the disclosure, the purity of LNnT in the whole broth and/or supernatant of at least 80% measured on the total amount of LNnT and LN3 produced by the cell in the whole broth and/or supernatant is obtained with the cell cultivated in a cultivation process, preferably a fermentation process.

A purity of LNnT of at least 80 percent on the total amount of LNnT and LN3 in the whole broth or in the supernatant should be understood as the amount of the LNnT in the mixture of LNnT and LN3 in the whole broth or in the supernatant, respectively, of 80% or more, comprising 80, 81, 82, 83, 84, 85, 86, 87, 88, 89, 90, 91, 92, 93, 94, 95, 95.5, 96, 96.5, 97, 97.5, 98, 98.5, 99 or 99.5% of LNnT measured on the total amount of LNnT and LN3 produced by the cell in the whole broth or in the supernatant, respectively.

According to another preferred embodiment of the method and/or cell of the disclosure, the cell is capable to synthesize a nucleotide-activated sugar to be used in the production of the oligosaccharide comprising a lacto-N-triose (LN3; GlcNAc-beta1,3-Gal-beta1,4-Glc) as a core trisaccharide. In a preferred embodiment of the method and/or cell of the disclosure, the nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.

Additionally, or alternatively, the host cell used herein is optionally genetically modified to express the de novo synthesis of GDP-fucose. The GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the host cell used herein is optionally genetically modified to express the de novo synthesis of CMP-Neu5Ac. The CMP-Neu5Ac can be provided by an enzyme expressed in the cell or by the metabolism of the cell. Such cell producing CMP-Neu5Ac can express an enzyme converting, e.g., sialic acid, which is to be added to the cell, to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. The modification can be any one or more chosen from the group comprising knock-out of an N-acetylglucosamine-6-phosphate deacetylase, knock-out of a glucosamine-6-phosphate deaminase, over-expression of a sialate synthase encoding gene, and over-expression of an N-acetyl-D-glucosamine-2-epimerase encoding gene.

Additionally, or alternatively, the host cell used herein is optionally genetically modified to express the de novo synthesis of UDP-Gal. The UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more chosen from the group comprising knock-out of a bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

It is generally preferred that the cell's catabolic pathway for selected mono-, di- or oligosaccharides is at least partially inactivated, the mono-, di-, or oligosaccharides being involved in and/or required for the synthesis of an oligosaccharide with LN3 as a core trisaccharide.

According to another preferred embodiment of the method and/or cell of the disclosure, the oligosaccharide comprising LN3 as a core trisaccharide is a mammalian milk oligosaccharide or a Lewis-type antigen oligosaccharide comprising LN3 as a core trisaccharide.

According to another preferred embodiment of the method and/or cell of the disclosure, the cell is capable to synthesize a mixture of oligosaccharides comprising at least one oligosaccharide comprising LN3 as a core trisaccharide.

In a specific exemplary embodiment, the method of the disclosure provides the production of an oligosaccharide with LN3 as a core trisaccharide in high yield. The method comprises the step of culturing or fermenting, an in aqueous culture or fermentation medium containing lactose, a genetically modified cell, preferably an E. coli, more preferably an E. coli cell modified by knocking-out the genes LacZ and nagB genes. Even more preferably, additionally the E. coli lacY gene, a fructose kinase gene (frk) originating from Zymomonas mobilis and a sucrose phosphorylase (SP) originating from Bifidobacterium adolescentis can be knocked in into the genome and expressed constitutively. The constitutive promoters originate from the promoter library described by De Mey et al. (BMC Biotechnology, 2007) and Mutalik et al. (Nat. Methods 2013, No. 10, 354-360). Additionally, the modified E. coli cell has a recombinant gene that encodes a galactoside beta-1,3-N-acetylglucosaminyltransferase and another recombinant gene that encodes a glycosyltransferase that is capable to modify LN3 to synthesize an oligosaccharide of this disclosure. The cell furthermore comprises a recombinant gene that encodes the expression of any one of the membrane proteins as described herein.

According to another preferred embodiment of the method and/or cell of the disclosure, the cell is using a precursor for the synthesis of the oligosaccharide comprising LN3 as a core trisaccharide. In a preferred embodiment of the method and/or cell, the membrane protein is involved in the uptake of a precursor for the synthesis of the oligosaccharide comprising LN3 as a core trisaccharide. In another preferred embodiment, the cell is producing a precursor for the synthesis of the oligosaccharide comprising LN3 as a core trisaccharide.

Another embodiment of the disclosure provides for a method and a cell wherein a glycosylated product is produced in and/or by a microorganism chosen from the list comprising a bacterium, a yeast or a fungus, or a plant cell, animal cell, a non-human mammalian cell, an insect cell or a protozoan. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably this disclosure specifically relates to a mutated and/or transformed Escherichia coli strain as indicated above wherein the E. coli strain is a K12 strain. More specifically, this disclosure relates to a mutated and/or transformed Escherichia coli strain as indicated above wherein the K12 strain is E. coli MG1655. The latter bacterium belonging to the phylum Firmicutes belongs preferably to the Bacilli, preferably from the species Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Pichia (with members like e.g., Pichia pastoris, P. anomala, P. kluyveri), Komagataella, Hansunella, Kluyveromyces (with members like e.g., Kluyveromyces lactis, K. marxianus, K. thermotolerans), Yarrowia (like e.g., Yarrowia lipolytica), Eremothecium, Zygosaccharomyces, Starmerella (like e.g., Starmerella bombicola) or Debaromyces. The latter yeast is preferably selected from Pichia pastoris, Yarrowia lipolitica, Saccharomyces cerevisiae and Kluyveromyces lactis. The latter fungus belongs preferably to the genus Rhizopus, Dictyostelium, Penicillium, Mucor or Aspergillus. “Plant cells” includes cells of flowering and non-flowering plants, as well as algal cells, for example, Chlamydomonas, Chlorella, etc. Preferably, the plant cell is a tobacco, alfalfa, rice, cotton, rapeseed, tomato, corn, maize or soybean cell. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641. The insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In a preferred embodiment the cell is a cell of a microorganism, wherein more preferably the microorganism is a bacterium or a yeast. In a more preferred embodiment, the microorganism is a bacterium, most preferably Escherichia coli. Examples using such E. coli are described herein.

In another more preferred embodiment, the cell is a yeast.

Another embodiment provides for a cell to be stably cultured in a medium, wherein the medium can be any type of growth medium comprising minimal medium, complex medium or growth medium enriched in certain compounds like, for example, but not limited to vitamins, trace elements, amino acids.

The microorganism or cell as used herein is capable to grow on a monosaccharide, disaccharide, oligosaccharide, polysaccharide, polyol, a complex medium or a mixture thereof as the main carbon source. With the term main is meant the most important carbon source for the oligosaccharide of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, maltose, lactose, arabinose, malto-oligosaccharides, maltotriose, sorbitol, xylose, rhamnose, sucrose, galactose, mannose, methanol, ethanol, trehalose, starch, cellulose, hemi-cellulose, corn-steep liquor, high-fructose syrup, acetate, citrate, lactate and pyruvate. With the term complex medium is meant a medium for which the exact constitution is not determined. Examples are molasses, corn steep liquor, peptone, tryptone or yeast extract.

In a further preferred embodiment, the microorganism or cell described herein is using a split metabolism having a production pathway and a biomass pathway as described in WO2012/007481, which is herein incorporated by reference. The organism can be, for example, genetically modified to accumulate fructose-6-phosphate by altering the genes selected from the phosphoglucoisomerase gene, phosphofructokinase gene, fructose-6-phosphate aldolase gene, fructose isomerase gene, and/or fructose:PEP phosphotransferase gene.

In a further preferred embodiment, the method for the production of an oligosaccharide with LN3 as a core trisaccharide as described herein comprises at least one of the following steps:
i) Adding to the culture medium a lactose feed comprising at least 50, more preferably at least 75, more preferably at least 100, more preferably at least 120, more preferably at least 150 gram of lactose per liter of initial reactor volume wherein the total reactor volume ranges from 250 mL (milliliter) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and
wherein preferably the temperature of the feed solution is kept between 20° C. and 80° C.;

the method resulting in a concentration of the oligosaccharide with LN3 as a core trisaccharide at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.

Preferably, the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.

In another embodiment the lactose feed is accomplished by adding lactose to the cultivation medium in a concentration, such that throughout the production phase of the cultivation a lactose concentration of at least 5 mM, preferably 10 mM or 30 mM is obtained.

In a further embodiment of the methods described herein the host cells are cultivated for at least about 60, 80, 100, or about 120 hours or in a continuous manner.

In another embodiment of the methods described herein a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate, pyruvate and/or lactose, is also added, preferably continuously to the culture medium, preferably with the lactose.

In a preferred embodiment, a carbon-based substrate is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per liter of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon-based substrate, preferably glucose or sucrose, to the culture medium before the lactose is added to the culture medium in a second phase.

In an alternative preferable embodiment, in the method as described herein, the lactose is added already in the first phase of exponential growth together with the carbon-based substrate.

According to this disclosure, the method as described herein preferably comprises a step of separating the oligosaccharide comprising LN3 as a core trisaccharide or oligosaccharide mixture comprising at least one oligosaccharide comprising LN3 as a core trisaccharide, respectively, from the cultivation.

The terms “separating from the cultivation” means harvesting, collecting, or retrieving the oligosaccharide or the oligosaccharide mixture comprising at least one oligosaccharide comprising LN3 as a core trisaccharide from the cell and/or the medium of its growth.

The oligosaccharide comprising LN3 as a core trisaccharide or oligosaccharide mixture comprising at least one oligosaccharide comprising LN3 as a core trisaccharide can be separated in a conventional manner from the aqueous culture medium, in which the cell was grown. In case the oligosaccharide or the oligosaccharide mixture is still present in the cells producing the oligosaccharide or the oligosaccharide mixture, conventional manners to free or to extract the oligosaccharide or the oligosaccharide mixture out of the cells can be used, such as cell destruction using high pH, heat shock, sonication, French press, homogenization, enzymatic hydrolysis, chemical hydrolysis, solvent hydrolysis, detergent, hydrolysis, . . . The culture medium and/or cell extract together and separately can then be further used for separating the oligosaccharide or the oligosaccharide mixture.

This preferably involves clarifying the oligosaccharide or the oligosaccharide mixture to remove suspended particulates and contaminants, particularly cells, cell components, insoluble metabolites and debris produced by culturing the genetically modified cell. In this step, the oligosaccharide or the oligosaccharide mixture can be clarified in a conventional manner. Preferably, the oligosaccharide or the oligosaccharide mixture is clarified by centrifugation, flocculation, decantation and/or filtration. A second step of separating the oligosaccharide or oligosaccharide mixture comprising at least one oligosaccharide comprising LN3 as a core trisaccharide preferably involves removing substantially all the proteins, as well as peptides, amino acids, RNA and DNA and any endotoxins and glycolipids that could interfere with the subsequent separation step, from the oligosaccharide or the oligosaccharide mixture, preferably after it has been clarified. In this step, proteins and related impurities can be removed from the oligosaccharide or the oligosaccharide mixture in a conventional manner. Preferably, proteins, salts, by-products, color and other related impurities are removed from the oligosaccharide or the oligosaccharide mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane.

In a further preferred embodiment, the methods as described herein also provide for a further purification of the oligosaccharide comprising LN3 as a core trisaccharide or the oligosaccharide mixture comprising at least one oligosaccharide comprising LN3 as a core trisaccharide of this disclosure. A further purification of the oligosaccharide or the oligosaccharide mixture may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the oligosaccharide or the oligosaccharide mixture. Another purification step is to dry, e.g., spray dry or lyophilize the produced oligosaccharide or oligosaccharide mixture.

In an exemplary embodiment, the separation and purification of the oligosaccharide comprising LN3 as a core trisaccharide or the oligosaccharide mixture comprising at least one oligosaccharide comprising LN3 as a core trisaccharide is made in a process, comprising the following steps in any order:
a) contacting the cultivation or a clarified version thereof with a nanofiltration membrane with a molecular weight cut-off (MWCO) of 600-3500 Da ensuring the retention of the produced oligosaccharide or oligosaccharide mixture and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the oligosaccharide or the oligosaccharide mixture in the form of a salt from the cation of the electrolyte.

In an alternative exemplary embodiment, the separation and purification of the oligosaccharide or the oligosaccharide mixture is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the oligosaccharide or the oligosaccharide mixture is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the oligosaccharide or the oligosaccharide mixture is made in the following way. The cultivation comprising the produced oligosaccharide, biomass, medium components and contaminants, and wherein the purity of the produced oligosaccharide or oligosaccharide mixture in the cultivation is <80 percent, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced oligosaccharide or oligosaccharide mixture at a purity of greater than or equal to 80 percent is provided. Optionally the purified solution is spray dried.

In an alternative exemplary embodiment, the separation and purification of the oligosaccharide comprising LN3 as a core trisaccharide or oligosaccharide mixture comprising at least one oligosaccharide comprising LN3 as a core trisaccharide is made in a process, comprising the following steps in any order: enzymatic treatment of the cultivation; removal of the biomass from the cultivation; ultrafiltration; nanofiltration; and a column chromatography step. Preferably, such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, this disclosure provides the produced oligosaccharide comprising LN3 as a core trisaccharide or oligosaccharide mixture comprising at least one oligosaccharide comprising LN3 as a core trisaccharide that is spray-dried to powder, wherein the spray-dried powder contains <15 percent-wt. of water, preferably <10 percent-wt. of water, more preferably <7 percent-wt. of water, most preferably <5 percent-wt. of water.

Another aspect of this disclosure provides for the use of a membrane protein selected from the group of membrane proteins as defined herein in the fermentative production of an oligosaccharide with LN3 as a core trisaccharide or an oligosaccharide mixture comprising at least one oligosaccharide comprising LN3 as a core trisaccharide.

In a further aspect, this disclosure provides for the use of a cell as defined herein, in a method for the production of an oligosaccharide with LN3 as a core trisaccharide or an oligosaccharide mixture comprising at least one oligosaccharide comprising LN3 as a core trisaccharide.

Furthermore, the disclosure also relates to the oligosaccharide with LN3 as a core trisaccharide or the oligosaccharide mixture comprising at least one oligosaccharide comprising LN3 as a core trisaccharide obtained by the methods according to the disclosure, as well as to the use of a polynucleotide, the vector, host cells, microorganisms or the polypeptide as described above for the production of the oligosaccharide or the oligosaccharide mixture. The oligosaccharide or the oligosaccharide mixture may be used as food additive, prebiotic, symbiotic, for the supplementation of baby food, adult food or feed, or as either therapeutically or pharmaceutically active compound. With the novel methods, the oligosaccharide with LN3 as a core trisaccharide or the oligosaccharide mixture comprising at least one oligosaccharide comprising LN3 as a core trisaccharide can easily and effectively be provided, without the need for complicated, time and cost consuming synthetic processes.

For identification of the oligosaccharide produced in the cell as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the oligosaccharide methods such as e.g., acid-catalyzed hydrolysis, HPLC (high performance liquid chromatography) or GLC (gas-liquid chromatography) (after conversion to alditol acetates) may be used. To determine the glycosidic linkages, the oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

The separated and preferably also purified oligosaccharide with LN3 as a core trisaccharide or oligosaccharide mixture comprising at least one oligosaccharide with LN3 as a core trisaccharide as described herein is incorporated into a food (e.g., human food or feed), dietary supplement, pharmaceutical ingredient, cosmetic ingredient or medicine. In some embodiments, the oligosaccharide or oligosaccharide mixture is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the oligosaccharide or oligosaccharide mixture being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, an oligosaccharide or oligosaccharide mixture produced and/or purified by a process of this specification is orally administered in combination with such microorganism.

Examples of further ingredients for dietary supplements include disaccharides (such as lactose), monosaccharides (such as glucose and galactose), thickeners (such as gum arabic), acidity regulators (such as trisodium citrate), water, skimmed milk, and flavorings.

In some embodiments, the oligosaccharide comprising LN3 as a core trisaccharide or the oligosaccharide mixture comprising at least one oligosaccharide comprising LN3 as a core trisaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, an oligosaccharide or oligosaccharide mixture produced and/or purified by a process in this specification is included in infant formula to provide nutritional benefits similar to those provided by the oligosaccharides in human breast milk. In some embodiments, the oligosaccharide or oligosaccharide mixture is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N— fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the oligosaccharide's or oligosaccharide mixture's concentration in the infant formula is approximately the same concentration as the oligosaccharide's concentration generally present in human breast milk.

In some embodiments, the oligosaccharide or oligosaccharide mixture is incorporated into a feed preparation, wherein the feed is chosen from the list comprising pet food, animal milk replacer, veterinary product, post weaning feed, or creep feed.

As will be shown in the examples herein, the method and the cell of the disclosure provide at least one of the following surprising advantages when using the membrane proteins as defined herein:
Better titers of the oligosaccharide with LN3 as a core trisaccharide (enhanced) (g/L),
   Better production rate r (g oligosaccharide/L/h),
   Better cell performance index CPI (g oligosaccharide/g X),
   Better specific productivity Qp (g oligosaccharide/g X/h),
   Better yield on sucrose Ys (g oligosaccharide/g sucrose),
   Better sucrose uptake/conversion rate Qs (g sucrose/g X/h),
   Better lactose conversion/consumption rate rs (g lactose/h),
   Enhanced secretion of the oligosaccharide with LN3 as a core trisaccharide, and/or
   Enhanced growth speed of the production host,
   when compared to a production host for an oligosaccharide with LN3 as a core trisaccharide of for an oligosaccharide mixture comprising at least one oligosaccharide comprising LN3 as a core trisaccharide with an identical genetic background but lacking the expression of the heterologous membrane protein or modulated expression of the endogenous membrane protein. In the present context, “X” means biomass, “g” means gram, “L” means liter and “h” means hour. The “g oligosaccharide” can be measured in the whole broth and/or in the supernatants.

Preferably, the method and the cell of the disclosure provide at least one of the following surprising advantages when using the membrane proteins as defined herein:
Better titers of the oligosaccharide with LN3 as a core trisaccharide (enhanced) (g/L),
Better production rate r (g oligosaccharide/L/h),
Better cell performance index CPI (g oligosaccharide/g X),
Better specific productivity Qp (g oligosaccharide/g X/h),
Better yield on sucrose Ys (g oligosaccharide/g sucrose),
Better sucrose uptake/conversion rate Qs (g sucrose/g X/h),
Better lactose conversion/consumption rate rs (g lactose/h), and/or
Enhanced secretion of the oligosaccharide with LN3 as a core trisaccharide,
when compared to a production host for an oligosaccharide with LN3 as a core trisaccharide of for an oligosaccharide mixture comprising at least one oligosaccharide comprising LN3 as a core trisaccharide with an identical genetic background but lacking the expression of the heterologous membrane protein or modulated expression of the endogenous membrane protein.

Moreover, this disclosure relates to the following specific embodiments:
1. Host cell genetically modified for the production of an oligosaccharide comprising a lacto-N-triose (LN3; GlcNAc-beta1,3-Gal-beta1,4-Glc) as a core trisaccharide, wherein the host cell comprises and expresses at least one nucleic acid sequence for a galactoside beta-1,3-N-acetylglucosaminyltransferase that transfers an N-acetylglucosamine (GlcNAc) residue from a UDP-GlcNAc donor to a lactose acceptor thereby synthesizing LN3,
   the cell further comprising i) overexpression of an endogenous membrane protein and/or ii) expression of a heterologous membrane protein providing a) improved production, and/or b) enabled and/or enhanced efflux, of an oligosaccharide comprising LN3 as a core trisaccharide,
   preferably the cell further comprising and expressing at least one nucleic acid sequence coding for a glycosyltransferase that is capable to modify the LN3.
2. Cell according to embodiment 1, wherein the membrane protein is selected from the group of porters, P-P-bond-hydrolysis-driven transporters, and β-Barrel Porins, wherein
   a) when the membrane protein is selected from the group of porters, the membrane protein is selected from
      the group of TCDB classes 2.A.1.1, 2.A.1.2, 2.A.1.3, 2.A.1.6, 2.A.2.2, 2.A.7.1 and 2.A.66, or
      the group of eggnog families 05E8G, 05EGZ, 05JHE, 07QF7 07QRN, 07RBJ, 0814C and 08N8A, or
      the PFAM list PF00893, PF01943, PF05977, PF07690 and PF13347, or
      the interpro list IPR000390, IPR001411, IPR001927, IPR002797, IPR004638, IPR005829, IPR010290, IPR011701, IPR020846, IPR023721, IPR023722, IPR032896, IPR036259 and IPR039672, or
      MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64 or MdfA from Kluyvera ascorbata ATCC 33433 with SEQ ID NO: 65, or functional homolog or functional fragment of any one of the above porter membrane proteins or a protein sequence having at least 80% sequence identity to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64 or 65, respectively;
   b) when the membrane protein is selected from the group of P-P-bond-hydrolysis-driven transporters, the membrane protein is selected from
      the group of TCDB classes 3.A.1.1 and 3.A.1.2, or
      the group of eggnog families 05CJ1, 05DFW, 05EZD, 05I1K, 07HR3 and 08IJ9, or
      the PFAM list PF00005, PF00528, PF13407 and PF17912, or
      the interpro list IPR000515, IPR003439, IPR003593, IPR005978, IPR008995, IPR013456, IPR015851, IPR017871, IPR025997, IPR027417, IPR028082, IPR035906, and IPR040582, or
      Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or functional homolog or functional fragment of any one of the above P-P-bond-hydrolysis-driven transporter membrane proteins or a protein sequence having at least 80% sequence identity to the full-length sequence of any one of the membrane proteins with SEQ ID NOs: 15, 16, 17, 18, 19 or 20, respectively; or
   c) when the membrane protein is selected from the group of β-Barrel Porins, the membrane protein is selected from
      TCDB class 1.B.18, or
      eggnog family 05DAY, or
      PFAM list PF02563, PF10531 and PF18412, or
      the interpro list IPR003715, IPR019554 and IPR040716, or
      Wza from Escherichia coli K12 MG1655 with SEQ ID NO: 21 or functional homolog or functional fragment thereof or a protein sequence having at least 80% sequence identity to the full-length sequence of the membrane protein with SEQ ID NO: 21;
      wherein the TCDB classes are as defined by TCDB.org as released on 17 Jun. 2019, the eggnog families are as defined by eggnogdb 4.5.1 as released on September 2016, the PFAM lists are as defined by Pfam 32.0 as released on September 2018, the interpro lists are as defined by InterPro 75.0 as released on 4 Jul. 2019.
3. Cell according to any one of embodiment 1 or 2, wherein the membrane protein is selected from the group of membrane proteins comprising
   a) the porter membrane proteins MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64 or MdfA from Kluyvera ascorbata ATCC 33433 with SEQ ID NO: 65, or functional homolog or functional fragment of any one of the above porter membrane proteins or a protein sequence having at least 80% sequence identity to the full-length sequence of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64 or 65, respectively; and
   b) the P-P-bond-hydrolysis-driven transporters Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or functional homolog or functional fragment of any one of the above P-P-bond-hydrolysis-driven transporter membrane proteins or a protein sequence having at least 80% sequence identity to the full-length sequence of the membrane proteins with SEQ ID NOs: 15, 16, 17, 18, 19 or 20, respectively; and
   c) the β-barrel porin membrane protein Wza from Escherichia coli K12 MG1655 with SEQ ID NO: 21 or functional homolog or functional fragment of the Wza protein or a protein sequence having at least 80% sequence identity to the full-length sequence of the membrane protein with SEQ ID NO: 21.
4. Cell according to any one of embodiments 1 to 3, wherein the membrane protein is a transporter protein involved in transport of compounds across the outer membrane of the cell wall.
5. Cell according to any one of previous embodiments, wherein the glycosyltransferase is selected from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, wherein the cell is modified in the expression or activity of at least one of the glycosyltransferases.
6. Cell according to any one of previous embodiments, wherein the oligosaccharide comprising a lacto-N-triose (LN3; GlcNAc-beta1,3-Gal-beta1,4-Glc) as a core trisaccharide is chosen from the list comprising lacto-N-triose, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, lacto-N-hexaose (LNH), lacto-N-neohexaose (LNnH), para-lacto-N-hexaose (pLNnH), para-lacto-N-neohexaose (pLNH), difucosyl-lacto-N-hexaose, difucosyl-lacto-N-neohexaose, lacto-N-pentaose (LNP), lacto-N-neopentaose, para lacto-N-pentaose, para lacto-N-neopentaose, lacto-N-novopentaose I, lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, Sialyl-lacto-N-tetraose a, Sialyl-lacto-N-tetraose b, Sialyl-lacto-N-tetraose c, Sialyl-lacto-N-tetraose d.
7. Cell according to any one of previous embodiments, wherein the glycosyltransferase is an N-acetylglucosamine beta-1,3-galactosyltransferase or an N-acetylglucosamine beta-1,4-galactosyltransferase that transfers a galactose (Gal) from a UDP-Gal donor to the terminal GlcNAc residue of LN3 in a beta-1,3 or beta-1,4 linkage, thereby producing lacto-N-tetraose (LNT; Gal-beta1,3-GlcNAc-beta1,3-Gal-beta1,4-Glc) or lacto-N-neotetraose (LNnT; Gal-beta1,4-GlcNAc-beta1,3-Gal-beta1,4-Glc), respectively.
8. Cell according to embodiment 7, wherein the cell produces 90 g/L or more of LNT in the whole broth and/or the supernatant and/or wherein the LNT in the whole broth and/or the supernatant has a purity of at least 80% measured on the total amount of LNT and LN3 produced by the cell in the whole broth and/or the supernatant, respectively.
9. Cell according to embodiment 7, wherein the cell produces 90 g/L or more of LNnT in the whole broth and/or the supernatant and/or wherein the LNnT in the whole broth and/or the supernatant has a purity of at least 80% measured on the total amount of LNnT and LN3 produced by the cell in the whole broth and/or the supernatant, respectively.
10. Cell according to any one of previous embodiments, wherein the cell is further capable to synthesize a nucleotide-activated sugar to be used in the production of the oligosaccharide comprising a lacto-N-triose (LN3; GlcNAc-beta1,3-Gal-beta1,4-Glc) as a core trisaccharide.
11. Cell according to embodiment 10, wherein the nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
12. Cell according to any one of previous embodiments, wherein the cell comprises a catabolic pathway for selected mono-, di- or oligosaccharides that is at least partially inactivated, the mono-, di-, or oligosaccharides being involved in and/or required for the synthesis of an oligosaccharide comprising LN3 as a core trisaccharide.
13. Cell according to any one of previous embodiments, wherein the cell is using a precursor for the synthesis of the oligosaccharide comprising LN3 as a core trisaccharide.
14. Cell according to any one of previous embodiments, wherein the membrane protein is involved in the uptake of a precursor for the synthesis of the oligosaccharide comprising LN3 as a core trisaccharide.
15. Cell according to any one of previous embodiments, wherein the cell is producing a precursor for the synthesis of the oligosaccharide comprising LN3 as a core trisaccharide.
16. Cell according to any one of previous embodiments, wherein the cell is stably cultured in a medium.
17. Cell according to any one of previous embodiments, wherein the cell is selected from the group comprising microorganism, plant, or animal cells, preferably the microorganism is a bacterium, fungus or a yeast, preferably the plant is a rice, cotton, rapeseed, soy, maize or corn plant, preferably the animal is an insect, fish, bird or non-human mammal.
18. Cell according to embodiment 17, wherein the cell is a cell of a bacterium, preferably of an Escherichia coli strain, more preferably of an Escherichia coli strain, which is a K-12 strain, even more preferably the Escherichia coli K-12 strain is E. coli MG1655.
19. Cell according to any one of previous embodiments, wherein the oligosaccharide comprising LN3 as a core trisaccharide is a mammalian milk oligosaccharide or a Lewis-type antigen oligosaccharide.
20. Cell according to any one of previous embodiments, wherein the cell is capable to synthesize a mixture of oligosaccharides comprising at least one oligosaccharide comprising LN3 as a core trisaccharide.
21. Method for the production of an oligosaccharide comprising a lacto-N-triose (LN3; GlcNAc-beta1,3-Gal-beta1,4-Glc) as a core trisaccharide by a genetically modified cell, comprising the steps of:
   a) providing a cell according to any one of the embodiments 1 to 20, and
   b) culturing the cell in a medium under conditions permissive for the production of the oligosaccharide comprising LN3 as a core trisaccharide,
   c) separating the oligosaccharide comprising LN3 as a core trisaccharide or oligosaccharide mixture comprising at least one oligosaccharide comprising LN3 as a core trisaccharide, respectively from the cultivation.
22. Method according to embodiment 21, the method further comprising at least one of the following steps:
   i) adding to the culture medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
   ii) adding a lactose feed in a continuous manner to the culture medium over the course of 1 day, 2 days, 3 days, 4 days, 5 days by means of a feeding solution;
   iii) adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
   the method resulting in an oligosaccharide comprising a lacto-N-triose (LN3; GlcNAc-beta1,3-Gal-beta1,4-Glc) as a core trisaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.
23. Method according to embodiment 22, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.
24. Method according to any one of embodiments 22 or 23, wherein the lactose feed is accomplished by adding lactose to the cultivation medium in a concentration, such, that throughout the production phase of the cultivation a lactose concentration of at least 5 mM, preferably 10 mM or 30 mM is obtained.
25. Method according to any one of embodiments 21 to 24, wherein the host cells are cultivated for at least about 60, 80, 100, or about 120 hours or in a continuous manner.
26. Method according to any one of embodiments 21 to 25, wherein a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the lactose.
27. Method according to any one of embodiments 21 to 26, wherein a first phase of exponential cell growth is provided by adding a carbon-based substrate, preferably glucose or sucrose, to the culture medium before the lactose is added to the culture medium in a second phase.
28. Method according to any one of embodiments 21 to 27, wherein the separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
29. Method according to any one of embodiments 21 to 28, further comprising purification of the oligosaccharide comprising LN3 as a core trisaccharide or oligosaccharide mixture comprising at least one oligosaccharide comprising LN3 as a core trisaccharide, respectively, from the cell.
30. Method according to any one of embodiments 21 to 29, wherein the purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
31. Use of a membrane protein selected from the group of membrane proteins as defined in any one of embodiments 1 to 4 in the fermentative production of an oligosaccharide comprising LN3 as a core trisaccharide.
32. Use of a cell according to any one of embodiments 1 to 19 for the production of an oligosaccharide comprising LN3 as a core trisaccharide.
33. Use of a cell according to embodiment 20 for the production of a mixture of oligosaccharides comprising at least one oligosaccharide comprising LN3 as a core trisaccharide.
34. Use of a method according to any one of embodiments 21 to 30 for the production of an oligosaccharide comprising LN3 as a core trisaccharide.

Moreover, this disclosure relates to the following preferred specific embodiments:
1. Host cell genetically modified for the production of an oligosaccharide comprising a lacto-N-triose (LN3; GlcNAc-beta1,3-Gal-beta1,4-Glc) as a core trisaccharide, wherein the host cell comprises and expresses at least one nucleic acid sequence for a galactoside beta-1,3-N-acetylglucosaminyltransferase that transfers an N-acetylglucosamine (GlcNAc) residue from a UDP-GlcNAc donor to a lactose acceptor thereby synthesizing LN3,
   the cell further comprising i) overexpression of an endogenous membrane protein and/or ii) expression of a heterologous membrane protein providing a) improved production, and/or b) enabled and/or enhanced efflux, of an oligosaccharide comprising LN3 as a core trisaccharide, preferably wherein the improved production and the enhanced efflux is compared to a host cell with an identical genetic background but lacking the overexpression of an endogenous membrane protein and the expression of a heterologous membrane protein,
   preferably the cell further comprising and expressing at least one nucleic acid sequence coding for a glycosyltransferase that is capable to modify the LN3,
   optionally wherein the improved production comprises:
   better titer of the oligosaccharide (gram oligosaccharide per liter),
   better production rate r (gram oligosaccharide per liter per hour),
   better cell performance index (gram oligosaccharide per gram biomass),
   better specific productivity (gram oligosaccharide per gram biomass per hour),
   better yield on sucrose (gram oligosaccharide per gram sucrose), and/or
   better sucrose uptake/conversion rate (gram sucrose per gram per hour),
   better lactose conversion/consumption rate (gram lactose per hour), and/or
   enhanced growth speed of the host cell.
2. Cell according to claim 1, wherein the membrane protein is selected from the group of porters, P-P-bond-hydrolysis-driven transporters, and β-Barrel Porins, wherein
   a) when the membrane protein is selected from the group of porters, the membrane protein is selected from
      the group of TCDB classes 2.A.1.1, 2.A.1.2, 2.A.1.3, 2.A.1.6, 2.A.2.2, 2.A.7.1 and 2.A.66, or
      the group of eggnog families 05E8G, 05EGZ, 05JHE, 07QF7 07QRN, 07RBJ, 0814C and 08N8A, or
      the PFAM list PF00893, PF01943, PF05977, PF07690 and PF13347, or
      the interpro list IPR000390, IPR001411, IPR001927, IPR002797, IPR004638, IPR005829, IPR010290, IPR011701, IPR020846, IPR023721, IPR023722, IPR032896, IPR036259 and IPR039672, or
      MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, MdfA from Enterobacter kobei with SEQ ID NO: 66, MdfA from Lelliottia sp. WB101 with SEQ ID NO: 67, MdfA from Citrobacter freundii with SEQ ID NO: 68, MdfA from Salmonella enterica subsp. Salamae with SEQ ID NO: 69 or MdfA from Shigella flexneri with SEQ ID NO: 70, or functional homolog or functional fragment of any one of the above porter membrane proteins, or a protein sequence having at least 80%, preferably at least 85%, more preferably at least 90%, even more preferably at least 95.00%, most preferably at least 97.00%, sequence identity to the full-length sequence of any one of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64, 65, 66, 67, 68, 69 or 70, respectively;
   b) when the membrane protein is selected from the group of P-P-bond-hydrolysis-driven transporters, the membrane protein is selected from
      the group of TCDB classes 3.A.1.1 and 3.A.1.2, or
      the group of eggnog families 05CJ1, 05DFW, 05EZD, 05I1K, 07HR3 and 08IJ9, or
      the PFAM list PF00005, PF00528, PF13407 and PF17912, or
      the interpro list IPR000515, IPR003439, IPR003593, IPR005978, IPR008995, IPR013456, IPR015851, IPR017871, IPR025997, IPR027417, IPR028082, IPR035906, and IPR040582, or
      Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or functional homolog or functional fragment of any one of the above P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of any one of the membrane proteins with SEQ ID NOs: 15, 16, 17, 18, 19 or 20, respectively; or
   c) when the membrane protein is selected from the group of β-Barrel Porins, the membrane protein is selected from
      TCDB class 1.B.18, or
      eggnog family 05DAY, or
      PFAM list PF02563, PF10531 and PF18412, or
      the interpro list IPR003715, IPR019554 and IPR040716, or
      Wza from Escherichia coli K12 MG1655 with SEQ ID NO: 21 or functional homolog or functional fragment thereof, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21;
      wherein the TCDB classes are as defined by TCDB.org as released on 17 Jun. 2019, the eggnog families are as defined by eggnogdb 4.5.1 as released on September 2016, the PFAM lists are as defined by Pfam 32.0 as released on September 2018, the interpro lists are as defined by InterPro 75.0 as released on 4 Jul. 2019.
3. Cell according to any one of claim 1 or 2, wherein the membrane protein is selected from the group of membrane proteins comprising
   a) the porter membrane proteins MdfA from Cronobacter muytjensii with SEQ ID NO: 01, MdfA from Yokenella regensburgei (ATCC43003) with SEQ ID NO: 02, MdfA from Escherichia coli K-12 MG1655 with SEQ ID NO: 03, MdfA from Enterobacter sp. with SEQ ID NO: 04, MFS from Citrobacter koseri with SEQ ID NO: 05, MdfA from Citrobacter youngae with SEQ ID NO: 06, YbdA from Escherichia coli K-12 MG1655 with SEQ ID NO: 07, YjhB from Escherichia coli K-12 MG1655 with SEQ ID NO: 08, WzxE from Escherichia coli K-12 MG1655 with SEQ ID NO: 09, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 017 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, MdfA from Enterobacter kobei with SEQ ID NO: 66, MdfA from Lelliottia sp. WB101 with SEQ ID NO: 67, MdfA from Citrobacter freundii with SEQ ID NO: 68, MdfA from Salmonella enterica subsp. Salamae with SEQ ID NO: 69 or MdfA from Shigella flexneri with SEQ ID NO: 70, or functional homolog or functional fragment of any one of the above porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64, 65, 66, 67, 68, 69 or 70, respectively; and
   b) the P-P-bond-hydrolysis-driven transporters Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or functional homolog or functional fragment of any one of the above P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein Wza from Escherichia coli K12 MG1655 with SEQ ID NO: 21 or functional homolog or functional fragment of the Wza protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
4. Cell according to any one of claims 1 to 3, wherein the membrane protein is selected from the group of membrane proteins comprising
   a) the porter membrane proteins represented by SEQ ID NO: 01, 02, 04, 05, 06, 09, 10, 11, 12, 13, 14, 53, 54, 55, 56, 57, 58, 59, 60, 61, 62, 63, 64, 65, 66, 67, 68, 69 or 70, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 05, 06, 09, 10, 11, 12, 13, 14, 53, 54, 55, 56, 57, 58, 59, 60, 61, 62, 63, 64, 65, 66, 67, 68, 69 or 70, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
5. Cell according to any one of claims 1 to 4, wherein the membrane protein is selected from the group of membrane proteins comprising
   a) the porter membrane proteins represented by SEQ ID NO: 01, 02, 04, 05, 06, 09, 10, 11, 12, 13, 14, 53, 54, 55, 56, 57, 58, 59, 60, 61, 62, 63, 64, 65, 66, 67, 68, 69 or 70, or functional homolog or functional fragment of any one of the porter membrane proteins, or a protein sequence having:
      at least 80% sequence identity to the full-length sequence of the membrane proteins with SEQ ID NOs: 09, 10, 11, 12 or 13, respectively,
      at least 90% sequence identity to the full-length sequence of the membrane proteins with SEQ ID NOs: 01, 02, 04, 14, 53, 54, 55, 59, 61, 62, 63, 64, 65, 66, 67 or 69, respectively,
      at least 95.00% sequence identity to the full-length sequence of the membrane proteins with SEQ ID NOs: 05, 06, 56, 57 or 68, respectively, or
      at least 99.00% sequence identity to the full-length sequence of the membrane proteins with SEQ ID NOs: 58, 60 or 70, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
6. Cell according to any one of claims 1 to 4, wherein the membrane protein is selected from the group of membrane proteins comprising
   a) the porter membrane proteins represented by SEQ ID NO: 01, 02, 04, 09, 10, 11, 12, 13, 14, 53, 54, 55, 59, 61, 62, 63, 64, 65, 66, 67, or 69, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64, 65, 66, 67, or 69, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
7. Cell according to any one of claims 1 to 4 or 6, wherein the membrane protein is selected from the group of membrane proteins comprising
   a) the porter membrane proteins represented by SEQ ID NO: 01, 02, 04, 09, 10, 11, 12, 13, 14, 53, 54, 55, 59, 61, 62, 63, 64, 65, 66, 67, or 69, or functional homolog or functional fragment of any one of the porter membrane proteins, or a protein sequence having at least 90%, preferably at least 95.00%, more preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64, 65, 66, 67, or 69, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
8. Cell according to any one of claims 1 to 3, wherein the membrane protein is selected from the group of membrane proteins comprising
   a) the porter membrane proteins represented by SEQ ID NO: 01, 02, 03, 04, 05, 06, 07, 08, 09, 10, 11, 12, 13, 14, 53, 54, 55, 56, 57, 58, 59, 60, 61, 62, 63, 64 or 65, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 03, 04, 05, 06, 07, 08, 09, 10, 11, 12, 13, 14, 53, 54, 55, 56, 57, 58, 59, 60, 61, 62, 63, 64 or 65, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
9. Cell according to any one of claims 1 to 3 or 8, wherein the membrane protein is selected from the group of membrane proteins comprising
   a) the porter membrane proteins represented by SEQ ID NO: 01, 02, 04, 05, 06, 09, 10, 11, 12, 13, 14, 53, 54, 55, 56, 57, 58, 59, 60, 61, 62, 63, 64 or 65, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 05, 06, 09, 10, 11, 12, 13, 14, 53, 54, 55, 56, 57, 58, 59, 60, 61, 62, 63, 64 or 65, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
10. Cell according to any one of claims 1 to 3, 8 or 9, wherein the membrane protein is selected from the group of membrane proteins comprising
   a) the porter membrane proteins represented by SEQ ID NO: 01, 02, 04, 05, 06, 09, 10, 11, 12, 13, 14, 53, 54, 55, 56, 57, 58, 59, 60, 61, 62, 63, 64 or 65, or functional homolog or functional fragment of any one of the porter membrane proteins, or a protein sequence having:
      at least 80% sequence identity to the full-length sequence of the membrane proteins with SEQ ID NOs: 09, 10, 11, 12 or 13, respectively,
      at least 90% sequence identity to the full-length sequence of the membrane proteins with SEQ ID NOs: 01, 02, 04, 14, 53, 54, 55, 59, 61, 62, 63, 64 or 65, respectively,
      at least 95.00% sequence identity to the full-length sequence of the membrane proteins with SEQ ID NOs: 05, 06, 56 or 57, respectively, or
      at least 99.00% sequence identity to the full-length sequence of the membrane proteins with SEQ ID NOs: 58 or 60, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
11. Cell according to any one of claims 1 to 3, 8 or 9, wherein the membrane protein is selected from the group of membrane proteins comprising
   a) the porter membrane proteins represented by SEQ ID NO: 01, 02, 04, 09, 10, 11, 12, 13, 14, 53, 54, 55, 59, 61, 62, 63, 64 or 65, or functional homolog or functional fragment of any one of the porter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64 or 65, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
12. Cell according to any one of claims 1 to 3, 8, 9 or 11, wherein the membrane protein is selected from the group of membrane proteins comprising
   a) the porter membrane proteins represented by SEQ ID NO: 01, 02, 04, 09, 10, 11, 12, 13, 14, 53, 54, 55, 59, 61, 62, 63, 64 or 65, or functional homolog or functional fragment of any one of the porter membrane proteins, or a protein sequence having at least 90%, preferably at least 95.00%, more preferably at least 97.00%, sequence identity to the full-length sequence of the membrane proteins with SEQ ID NOs: 01, 02, 04, 09, 10, 11, 12, 13, 14, 53, 54, 55, 59, 61, 62, 63, 64 or 65, respectively; and
   b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, preferably at least 85%, more preferably at least 90%, even more preferably at least 95.00%, most preferably at least 97.00%, sequence identity to the full-length sequence of the membrane proteins with SEQ ID NOs: 15, 16, 17, 18, 19 or 20, respectively; and
   c) the β-barrel porin membrane protein represented by SEQ ID NO: 21, or functional homolog or functional fragment of the β-barrel porin membrane protein, or a protein sequence having at least 80%, preferably at least 85%, more preferably at least 90%, even more preferably at least 95.00%, most preferably at least 97.00%, sequence identity to the full-length sequence of the membrane protein with SEQ ID NO: 21.
13. Cell according to any one of claims 1 to 12, wherein the membrane protein is a transporter protein involved in transport of compounds across the outer membrane of the cell wall.
14. Cell according to any one of claims 1 to 13, wherein the glycosyltransferase is selected from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, wherein the cell is modified in the expression or activity of at least one of the glycosyltransferases.
15. Cell according to any one of claims 1 to 14, wherein the oligosaccharide comprising a lacto-N-triose (LN3; GlcNAc-beta1,3-Gal-beta1,4-Glc) as a core trisaccharide is a mammalian milk oligosaccharide or a Lewis-type antigen oligosaccharide.
16. Cell according to any one of claims 1 to 15, wherein the oligosaccharide comprising a lacto-N-triose (LN3; GlcNAc-beta1,3-Gal-beta1,4-Glc) as a core trisaccharide is a mammalian milk oligosaccharide (MMO), preferably a human milk oligosaccharide (HMO), more preferably a MMO or HMO having a LNT or LNnT as a core tetrasaccharide, even more preferably a HMO having a LNT or LNnT as a core tetrasaccharide, most preferably LNT or LNnT.
17. Cell according to any one of claims 1 to 16, wherein the oligosaccharide comprising a lacto-N-triose (LN3; GlcNAc-beta1,3-Gal-beta1,4-Glc) as a core trisaccharide is a neutral oligosaccharide.
18. Cell according to any one of claims 1 to 17, wherein the oligosaccharide comprising a lacto-N-triose (LN3; GlcNAc-beta1,3-Gal-beta1,4-Glc) as a core trisaccharide is chosen from the list comprising lacto-N-triose, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, lacto-N-hexaose (LNH), lacto-N-neohexaose (LNnH), para-lacto-N-hexaose (pLNnH), para-lacto-N-neohexaose (pLNH), difucosyl-lacto-N-hexaose, difucosyl-lacto-N-neohexaose, lacto-N-pentaose (LNP), lacto-N-neopentaose, para lacto-N-pentaose, para lacto-N-neopentaose, lacto-N-novopentaose I, lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, Sialyl-lacto-N-tetraose a, Sialyl-lacto-N-tetraose b, Sialyl-lacto-N-tetraose c, Sialyl-lacto-N-tetraose d.
19. Cell according to any one of claims 1 to 18, wherein the glycosyltransferase is an N-acetylglucosamine beta-1,3-galactosyltransferase or an N-acetylglucosamine beta-1,4-galactosyltransferase that transfers a galactose (Gal) from a UDP-Gal donor to the terminal GlcNAc residue of LN3 in a beta-1,3 or beta-1,4 linkage, thereby producing lacto-N-tetraose (LNT; Gal-beta1,3-GlcNAc-beta1,3-Gal-beta1,4-Glc) or lacto-N-neotetraose (LNnT; Gal-beta1,4-GlcNAc-beta1,3-Gal-beta1,4-Glc), respectively.
20. Cell according to claim 19, wherein the cell produces 90 g/L or more of LNT in the whole broth and/or the supernatant and/or wherein the LNT in the whole broth and/or the supernatant has a purity of at least 80% measured on the total amount of LNT and LN3 produced by the cell in the whole broth and/or the supernatant, respectively.
21. Cell according to claim 19, wherein the cell produces 70 g/L or more, preferably 90 g/L or more, of LNnT in the whole broth and/or the supernatant and/or wherein the LNnT in the whole broth and/or the supernatant has a purity of at least 80% measured on the total amount of LNnT and LN3 produced by the cell in the whole broth and/or the supernatant, respectively.
22. Cell according to any one of claims 1 to 12, wherein the cell is further capable to synthesize a nucleotide-activated sugar to be used in the production of the oligosaccharide comprising a lacto-N-triose (LN3; GlcNAc-beta1,3-Gal-beta1,4-Glc) as a core trisaccharide.
23. Cell according to claim 22, wherein the nucleotide-activated sugar is chosen from the list comprising UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-N-glycolylneuraminic acid (CMP-Neu5Gc), GDP-fucose (GDP-Fuc), GDP-rhamnose and UDP-xylose.
24. Cell according to any one of claims 1 to 23, wherein the cell comprises a catabolic pathway for selected mono-, di- or oligosaccharides that is at least partially inactivated, the mono-, di-, or oligosaccharides being involved in and/or required for the synthesis of an oligosaccharide comprising LN3 as a core trisaccharide.
25. Cell according to any one of claims 1 to 24, wherein the cell is using a precursor for the synthesis of the oligosaccharide comprising LN3 as a core trisaccharide.
26. Cell according to any one of claims 1 to 25, wherein the membrane protein is involved in the uptake of a precursor for the synthesis of the oligosaccharide comprising LN3 as a core trisaccharide.
27. Cell according to any one of claims 1 to 26, wherein the cell is producing a precursor for the synthesis of the oligosaccharide comprising LN3 as a core trisaccharide.
28. Cell according to any one of claims 1 to 27, wherein the cell is stably cultured in a medium.
29. Cell according to any one of claims 1 to 28, wherein the cell is a microorganism, a plant cell, an animal cell, an insect cell or a protozoan cell, preferably wherein
the microorganism is a bacterium, fungus or a yeast,
the plant cell is a tobacco, alfalfa, rice, cotton, rapeseed, soy, maize or corn, and/or cell
the animal cell is derived from non-human mammals, birds, fish, invertebrates, reptiles, or amphibians, or the animal cell is a genetically modified cell line derived from human cells excluding embryonic stem cells.
30. Cell according to claim 29, wherein the cell is a cell of a bacterium, preferably of an Escherichia coli strain, more preferably of an Escherichia coli strain, which is a K-12 strain, even more preferably the Escherichia coli K-12 strain is E. coli MG1655.
31. Cell according to any one of claims 1 to 30, wherein the cell is capable to synthesize a mixture of oligosaccharides comprising at least one oligosaccharide comprising LN3 as a core trisaccharide.
32. Method for the production of an oligosaccharide comprising a lacto-N-triose (LN3; GlcNAc-beta1,3-Gal-beta1,4-Glc) as a core trisaccharide by a genetically modified cell, comprising the steps of:
   a) providing a cell according to any one of claims 1 to 31, and
   b) culturing the cell in a medium under conditions permissive for the production of the oligosaccharide comprising LN3 as a core trisaccharide, and
   c) preferably separating the oligosaccharide comprising LN3 as a core trisaccharide or oligosaccharide mixture comprising at least one oligosaccharide comprising LN3 as a core trisaccharide, respectively from the cultivation.
33. Method according to claim 32, the method further comprising at least one of the following steps:
   i) adding to the culture medium a lactose feed comprising at least 50, more preferably at least 75, more preferably at least 100, more preferably at least 120, more preferably at least 150 gram of lactose per liter of initial reactor volume wherein the reactor volume ranges from 250 mL to 10.000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
   ii) adding a lactose feed in a continuous manner to the culture medium over the course of 1 day, 2 days, 3 days, 4 days, 5 days by means of a feeding solution;
   iii) adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and wherein preferably the temperature of the feed solution is kept between 20° C. and 80° C.;
   the method resulting in an oligosaccharide comprising a lacto-N-triose (LN3; GlcNAc-beta1,3-Gal-beta1,4-Glc) as a core trisaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the culture medium.
34. Method according to claim 33, wherein the lactose feed is accomplished by adding lactose from the beginning of the cultivating in a concentration of at least 5 mM, preferably in a concentration of 30, 40, 50, 60, 70, 80, 90, 100, 150 mM, more preferably in a concentration >300 mM.
35. Method according to claim 33 or 34, wherein the lactose feed is accomplished by adding lactose to the cultivation medium in a concentration, such, that throughout the production phase of the cultivation a lactose concentration of at least 5 mM, preferably 10 mM or 30 mM is obtained.
36. Method according to any one of claims 32 to 35, wherein the host cells are cultivated for at least about 60, 80, 100, or about 120 hours or in a continuous manner.
37. Method according to any one of claims 32 to 36, wherein a carbon and energy source, preferably glucose, glycerol, fructose, maltose, arabinose, maltodextrines, malto-oligosaccharides, maltotriose, sorbitol, xylose, rhamnose, sucrose, galactose, mannose, methanol, ethanol, trehalose, starch, cellulose, hemi-cellulose, polyols, corn-steep liquor, high-fructose syrup, succinate, malate, acetate, citrate, lactate and pyruvate, is also added, preferably continuously to the culture medium, preferably with the lactose.
38. Method according to any one of claims 32 to 37, wherein a first phase of exponential cell growth is provided by adding a carbon-based substrate, preferably glucose or sucrose, to the culture medium before the lactose is added to the culture medium in a second phase.
39. Method according to any one of claims 32 to 38, wherein the separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography and/or gel filtration, ligand exchange chromatography.
40. Method according to any one of claims 32 to 39, further comprising purification of the oligosaccharide comprising LN3 as a core trisaccharide or oligosaccharide mixture comprising at least one oligosaccharide comprising LN3 as a core trisaccharide, respectively from the cell.
41. Method according to any one of claims 32 to 40, wherein the purification comprises at least one of the following steps: use of activated charcoal or carbon, use of charcoal, nanofiltration, ultrafiltration or ion exchange, use of alcohols, use of aqueous alcohol mixtures, crystallization, evaporation, precipitation, drying, spray drying or lyophilization.
42. Use of a membrane protein selected from the group of membrane proteins as defined in any one of claims 1 to 12 in the fermentative production of an oligosaccharide comprising LN3 as a core trisaccharide.
43. Use of a cell according to any one of claims 1 to 31 for the production of an oligosaccharide comprising LN3 as a core trisaccharide.
44. Use of a cell according to claim 31 for the production of a mixture of oligosaccharides comprising at least one oligosaccharide comprising LN3 as a core trisaccharide.
45. Use of a method according to any one of claims 32 to 41 for the production of an oligosaccharide comprising LN3 as a core trisaccharide.

The disclosure will be described in more detail in the examples. The examples will serve as further illustration and clarification of this disclosure and are not intended to be limiting.

### EXAMPLES

### Example 1. Identification of Membrane Protein Families

An HMM is a probabilistic model called profile hidden Markov models. It characterizes a set of aligned proteins into a position-specific scoring system. Amino acids are given a score at each position in the sequence alignment according to the frequency by which they occur (Eddy, S. R.1998. Profile hidden Markov models. Bioinformatics. 14: 755-63). HMMs have wide utility, as is clear from the numerous databases that use this method for protein classification, including Pfam, InterPro, SMART, TIGRFAM, PIRSF, PANTHER, SFLD, Superfamily and Gene3D.

HMMsearch from the HMMER package 3.2.1 (hmmer.org/) as released on 13 Jun. 2019 can use this HMM to search sequence databases for sequence homologs. Sequence databases that can be used are, for example, but not limited to: the NCBI nr Protein Database (NR; www.ncbi.nlm.nih.gov/protein), UniProt Knowledgebase (UniProtKB, www.uniprot.org/help/uniprotkb) and the SWISS-PROT database (web.expasy.org/docs/swiss-prot_guideline.html).

Membrane protein families were classified based on the eggNOG database 4.5.1 (www.ncbi.nlm.nih.gov/pmc/articles/PMC6324079/; eggnog.embl.de/#/app/home) as released on September 2016, the TCDB database (www.tcdb.org/public/tcdb) as released on 17 Jun. 2019, InterPro 75.0 (www.ebi.ac.uk/interpro/) as released on 4 Jul. 2019 and PFAM domains using Pfam 32.0 (pfam.xfam.org/) as released on September 2018. The eggNOG database is a public database of orthology relationships, gene evolutionary histories and functional annotations. The Transporter Classification DataBase (TCDB) is analogous to the Enzyme Commission (EC) system for classifying enzymes and incorporates both functional and phylogenetic information. The Pfam and InterPro databases are a large collection of protein families. Other protein domains like SMART (smart.embl-heidelberg.de/), TIGRFAM (www.jcvi.org/tigframs), PIRSF (proteinginformationresource.org/pirwww/dbinfo/pirsf.shtml), PANTHER (pantherdb.org/), SFLD (sfld.rbvi.ucsf.edu/archive/django/index.html), Superfamily (supfam.org/) and Gene3D (gene3d.biochem.ucl.ac.uk/Gene3D/), NCBI Conserved Domains (www.ncbi.nlm.nih.gov/Structure/cdd/wrpsb.cgi) can also be used.

Identification of eggNOG families was done by using a standalone version of eggNOG-mapper 1.0.3 (github.com/eggnogdb/eggnot-mapper) as released on Nov. 15, 2017 and as based on eggnogdb 4.5.1 as released in September 2016. For each of the eggNOG families an HMM can be downloaded on the eggNOG website and can be used for HMMsearch to the protein databases.

Identification of the TCDB family was done by blasting (blastp) to the TCDB database as released on 17 Jun. 2019. New members of the obtained family can be retrieved on the website (www.tcdb.org/download.php). Fasta files can be used as input in blastp to the protein databases.

Identification of the PFAM domains was done by an online search on pfam.xfam.org/search#tabview=tab1 as released on September 2018. The HMM for the obtained family was downloaded in ‘Curation & model.’ HMMsearches with this model to the protein databases will identify new family members. Sequences comprising the InterPro hit can also be downloaded from the PFAM website.

Identification of the InterPro (super)families, domains and sites was done by using the online tools on www.ebi.ac.uk/interpro/ or a standalone version of InterProScan (www.ebi.ac.uk/interpro/download.html), both based on InterPro 75.0 as released on 4 Jul. 2019. InterPro is a composite database combining the information of many databases of protein motifs and domains. The HMM of the InterPro domain and/or (super)families can be obtained from InterProScan and can be used to identify new family members in the protein databases. Sequences comprising the InterPro hit can also be downloaded from the InterPro website (‘Protein Matched’) or can be queried on the UniProt website (www.uniprot.org).

### Example 2: Calculation of Percentage Identity Between Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e., spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

### Example 3: Identification of Homologs of the Membrane Protein with SEQ ID NO: 01

Homologs of transporter genes can be obtained from sequence databases like PATRIC (www.patricbrc.org/), Uniprot (www.uniprot.org/), NCBI nr or nt databases (www.ncbi.nlm.nih.gov/) and others. PATRIC (www.patricbrc.org/) is an integration of different types of data and software tools that support research on bacterial pathogens.

This example describes how to extract genes encoding for protein sequences with at least 80% sequence identity to the full-length gene encoding SEQ ID NO: 01. The membrane protein with SEQ ID NO: 01 belongs to the global family PGF_00466006. Members of this family were extracted using the PATRIC command-line interface (docs.patricbrc.org/cli_tutorial/index.html). Amino acid sequences were filtered with >80% global sequence identity to SEQ ID NO: 01 as calculated using EMBOSS Needle with default parameters. 70477 identifiers representing 7002 unique sequences were extracted on 25 Nov. 2020. Blastp with default parameters and SEQ ID NO: 01 as query was used to extract sequences from Uniprot (database release data 2 Dec. 2020). Amino acid sequences were filtered with >80% global sequence identity to SEQ ID NO: 01 as calculated using EMBOSS Needle with default parameters and resulted in 1471 identifiers. Amongst these identifiers, the membrane proteins with SEQ ID NOs: 53, 54, 55, 56, 57, 58, 59, 60, 63, 64, 65, 66, 67 and 68, as enlisted in the attached sequence listing could be identified.

### Example 4. Materials and Methods Escherichia coli

Media

The Luria Broth (LB) medium comprised 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH4Cl, 5.00 g/L (NH4)2S04, 2.993 g/L KH2PO4, 7.315 g/L K2HPO4, 8.372 g/L MOPS, 0.5 g/L NaCl, 0.5 g/L MgSO4·7H2O, 30 g/L sucrose or another carbon source when specified in the examples, 1 ml/L vitamin solution, 100 μL/L molybdate solution, and 1 mL/L selenium solution. As specified in the respective examples, 20 g/L lactose was additionally added to the medium as precursor. The minimal medium was set to a pH of 7 with 1M KOH. Vitamin solution comprised 3.6 g/L FeCl2·4H2O, 5 g/L CaCl2·2H2O, 1.3 g/L MnCl2·2H2O, 0.38 g/L CuCl2·2H2O, 0.5 g/L CoCl2·6H2O, 0.94 g/L ZnCl2, 0.0311 g/L H3B04, 0.4 g/L Na2EDTA·2H2O and 1.01 g/L thiamine·HCl. The molybdate solution contained 0.967 g/L NaMoO4.2H2O. The selenium solution contained 42 g/L Seo2.

The minimal medium for fermentations contained 6.75 g/L NH4Cl, 1.25 g/L (NH4)2SO4, 2.93 g/L KH2PO4 and 7.31 g/L KH2PO4, 0.5 g/L NaCl, 0.5 g/L MgSO4·7H2O, 30 g/L sucrose, 1 mL/L vitamin solution, 100 μL/L molybdate solution, and 1 mL/L selenium solution with the same composition as described above. As specified in the respective examples, 100 g/L lactose was additionally added to the medium as precursor.

Complex medium was sterilized by autoclaving (121° C., 21′) and minimal medium by filtration (0.22 μm Sartorius). When necessary, the medium was made selective by adding an antibiotic (e.g., chloramphenicol (20 mg/L), carbenicillin (100 mg/L), spectinomycin (40 mg/L) and/or kanamycin (50 mg/L)).

Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZdeltaM15, delta(lacZYAargF) U169, deoR, recA1, endA1, hsdR17(rk, mk⁺), phoA, supE44, lambda, thi-1, gyrA96, relA1) bought from Invitrogen.

Strains and Mutations

Escherichia coli K12 MG1655 [lambda⁻, F⁻, rph-1] was obtained from the Coli Genetic Stock Center (US), CGSC Strain #: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD₆₀₀ₙₘ of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0). The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knockouts and knock-ins are checked with control primers.

To produce lacto-N-triose (LN3, LNT-II, GlcNAc-b1,3-Gal-b1,4-Glc) and oligosaccharides originating thereof comprising lacto-N-tetraose (LNT) and lacto-N-neotetraose (LNnT), the mutant strain was derived from E. coli K12 MG1655 and modified with a knock-out of the E. coli LacZ and nagB genes and with a genomic knock-in of a constitutive transcriptional unit for the galactoside beta-1,3-N-acetylglucosaminyltransferase (LgtA) from N. meningitidis with SEQ ID NO: 22. For LNT or LNnT production, the mutant strain is further modified with constitutive transcriptional units for the N-acetylglucosamine beta-1,3-galactosyltransferase (WbgO) from E. coli O55:H7 with SEQ ID NO: 23 or the N-acetylglucosamine beta-1,4-galactosyltransferase (LgtB) from N. meningitidis with SEQ ID NO: 24, respectively, that can be delivered to the strain either via genomic knock-in or from an expression plasmid. Optionally, multiple copies of the LgtA, wbgO and/or LgtB genes could be added to the mutant E. coli strains. In these strains, LNT and/or LNnT production can be enhanced by improved UDP-GlcNAc production by modification of the strains with one or more genomic knock-ins of a constitutive transcriptional unit for the mutant L-glutamine-D-fructose-6-phosphate aminotransferase glmS*54 from E. coli with SEQ ID NO: 25 (differing from the wild-type glmS by an A39T, an R250C and an G472S mutation). In addition, the strains can optionally be modified for enhanced UDP-galactose production with genomic knockouts of the E. coli ushA and galT genes. The mutant E. coli strains can also optionally be adapted with a genomic knock-in of a constitutive transcriptional unit for the UDP-glucose-4-epimerase (galE) from E. coli with SEQ ID NO: 26, the phosphoglucosamine mutase (glmM) from E. coli with SEQ ID NO: 27 and the N-acetylglucosamine-1-phosphate uridyltransferase/glucosamine-1-phosphate acetyltransferase (glmU) from E. coli with SEQ ID NO: 28.

For production of fucosylated oligsosaccharides with LN3 as a core trisaccharide, the E. coli strains were further modified with knockouts of the E. coli wcaJ and thyA genes and with expression plasmids comprising constitutive transcriptional units for the H. pylori alpha-1,2-fucosyltransferase with SEQ ID NO: 29 (HpFutC) and/or the H. pylori alpha-1,3-fucosyltransferase with SEQ ID NO: 30 (HpFucT) and with a constitutive transcriptional unit for the E. coli thyA with SEQ ID NO: 31 as selective marker. The constitutive transcriptional units of the fucosyltransferase genes could also be present in the mutant E. coli strain via genomic knock-ins. GDP-fucose production can further be optimized by genomic knockouts of the E. coli genes comprising glgC, agp, pfkA, pfkB, pgi, arcA, iclR, pgi and lon as described in WO2016075243 and WO2012007481. GDP-fucose production can additionally be optimized comprising genomic knock-ins of constitutive transcriptional units for the E. coli manA with SEQ ID NO: 32, manB with SEQ ID NO: 33, manC with SEQ ID NO: 34, gmd with SEQ ID NO: 35 and fcl with SEQ ID NO: 36. GDP-fucose production can also be obtained by genomic knockouts of the E. coli fucK and fucI genes and genomic knock-ins of constitutive transcriptional units containing the fucose permease (fucP) from E. coli with SEQ ID NO: 37 and the bifunctional fucose kinase/fucose-1-phosphate guanylyltransferase (fkp) from Bacteroides fragilis with SEQ NO: ID 38.

For production of sialylated oligosaccharides with LN3 as a core trisaccharide, the E. coli strains were further modified with a knockout of the E. coli nagA gene and genomic knock-ins of constitutive transcriptional units containing a glucosamine 6-phosphate N-acetyltransferase (GNA1) from Saccharomyces cerevisiae with SEQ ID NO: 39, an N-acetylglucosamine 2-epimerase (AGE) from Bacteroides ovatus with SEQ ID NO: 40, an N-acetylneuraminate (Neu5Ac) synthase (NeuB) from Neisseria meningitidis with SEQ ID NO: 41, an N-acylneuraminate cytidylyltransferase (NeuA) from Pasteurella multocida with SEQ ID NO: 42, and a beta-galactoside alpha-2,3-sialyltransferase comprising SEQ ID NO: 43 (PmultST3) from P. multocida and/or SEQ ID NO: 44 (NmeniST3) from N. meningitidis, and/or a beta-galactoside alpha-2,6-sialyltransferase comprising SEQ ID NO: 45 (PdST6) from Photobacterium damselae and/or SEQ ID NO: 46 (P-JT-ISH-224-ST6) from Photobacterium sp. JT-ISH-224. Constitutive transcriptional units of PmNeuA and the sialyltransferases can be delivered to the mutant strain either via genomic knock-in or via expression plasmids. Sialic acid production can further be optimized in the mutant E. coli strain with genomic knock-outs of the E. coli genes comprising nagC, nagD, nagE, nanA, nanE, nanK, manX, manY and manZ as described in WO18122225 and with genomic knock-ins of constitutive transcriptional units comprising a mutated variant of the L-glutamine-D-fructose-6-phosphate aminotransferase (glmS*54) from E. coli with SEQ ID NO: 25 (differing from the wild-type E. coli glmS by an A39T, an R250C and an G472S mutation) and the phosphatase yqaB from E. coli with SEQ ID NO: 47. Sialic acid production can also be obtained by knockouts of the E. coli nagA and nagB genes and genomic knock-ins of constitutive transcriptional units containing the phosphoglucosamine mutase (glmM) from E. coli with SEQ ID NO: 27, the N-acetylglucosamine-1-phosphate uridyltransferase/glucosamine-1-phosphate acetyltransferase (glmU) from E. coli with SEQ ID NO: 28, the UDP-N-acetylglucosamine 2-epimerase (NeuC) from Campylobacter jejuni with SEQ ID NO: 48 and the N-acetylneuraminate synthase (NeuB) from N. meningitidis with SEQ ID NO: 41. In this mutant strain, sialic acid production can further be optimized with genomic knock-ins of constitutive transcriptional units comprising the mutant glmS*54 from E. coli with SEQ ID NO: 25 and the phosphatase yqaB from E. coli with SEQ ID NO: 47.

All mutant strains could also optionally be adapted for growth on sucrose via genomic knock-ins of constitutive transcriptional units containing a sucrose transporter (CscB) from E. coli W with SEQ ID NO: 49, a fructose kinase (Frk) originating from Z. mobilis with SEQ ID NO: 50 and a sucrose phosphorylase originating from B. adolescentis with SEQ ID NO: 51. Furthermore, the mutant strains could be modified for enhanced lactose uptake via genomic knock-in of a constitutive transcriptional unit for the lactose permease lacY from E. coli with SEQ ID NO: 52.

Furthermore, all mutant strains could be optionally adapted for intracellular lactose synthesis by genomic knock-outs of lacZ, glk and the galETKM operon, together with genomic knock-ins of constitutive transcriptional units for lgtB from N. meningitidis with SEQ ID NO: 24 and the UDP-glucose 4-eprimerase (galE) from E. coli with SEQ ID NO: 26.

In a next step, the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 as enlisted in Tables 2 and 3 can be evaluated in above mutant strains derived from E. coli K12 MG1655. Other proteins described in this disclosure are also enlisted in Table 2. Membrane protein genes can be evaluated either present on a pSC101 plasmid or integrated in the host's genome in constitutive transcriptional units.

Preferably, but not necessarily, the glycosyltransferases were N-terminally fused to an MBP-tag to enhance their solubility (Fox et al., Protein Sci. 2001, 10(3), 622-630).

All constitutive promoters, UTRs and terminator sequences originated from the libraries described by Cambray et al. (Nucleic Acids Res. 2013, 41(9), 5139-5148), Chen et al. (Nat. Methods 2013, 10(7), 659-664), De Mey et al. (BMC Biotechnol. 2007, 4(34), 1-14), Dunn et al. (Nucleic Acids Res. 1980, 8(10), 2119-2132), Kim and Lee (FEBS Letters 1997, 407(3), 353-356) and Mutalik et al. (Nat. Methods 2013, No. 10, 354-360). All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

TABLE 2
Overview of SEQ ID NOs described in this disclosure

| | | | | Country of |
|---|---|---|---|---|
| | | | | origin of |
| SEQ | Name/ | | | digital |
| ID | TCDB | | | sequence |
| NO | group | Organism | Origin | information |
| 01 | MdfA | Cronobacter muytjensii | Synthetic | USA |
| 02 | MdfA | Yokenella regensburgei | Synthetic | Unknown |
| | | (ATCC43003) | | |
| 03 | MdfA | Escherichia coli | Synthetic | USA |
| | | K12 MG1655 | | |
| 04 | MdfA | Enterobacter sp. | Synthetic | Australia |
| 05 | MFS | Citrobacter koseri | Synthetic | USA |
| 06 | MdfA | Citrobacter youngae | Synthetic | USA |
| | | ATCC 29220 | | |
| 07 | YbdA | Escherichia coli | Synthetic | USA |
| | | K12 MG1655 | | |
| 08 | YjhB | Escherichia coli | Synthetic | USA |
| | | K12 MG1655 | | |
| 09 | WzXE | Escherichia coli | Synthetic | USA |
| | | K12 MG1655 | | |
| 10 | EmrE | Escherichia coli | Synthetic | USA |
| | | K12 MG1655 | | |
| 11 | Blon_ | Bifidobacterium longum | Synthetic | Germany |
| | 2331 | subsp. Infantis | | |
| | | (strain ATCC 15697) | | |
| 12 | Blon_ | Bifidobacterium longum | Synthetic | Germany |
| | 0247 | subsp. Infantis | | |
| | | (strain ATCC 15697) | | |
| 13 | Blon_ | Bifidobacterium longum | Synthetic | Germany |
| | 0345 | subsp. Infantis | | |
| | | (strain ATCC 15697) | | |
| 14 | IceT | Klebsiella pneumoniae | Synthetic | USA |
| 15 | Blon_ | Bifidobacterium longum | Synthetic | Germany |
| | 2475 | subsp. Infantis | | |
| | | (strain ATCC 15697) | | |
| 16 | nodi | Bradyrhizobium | Synthetic | USA |
| | | japonicum | | |
| | | USDA 110 | | |
| 17 | xylF | Escherichia coli | Synthetic | USA |
| | | K12 MG1655 | | |
| 18 | TIC77290 | Bifidobacterium longum | Synthetic | USA |
| | | infantis Bi-26 | | |
| 19 | TIC77291 | Bifidobacterium longum | Synthetic | USA |
| | | infantis Bi-26 | | |
| 20 | TIC76854 | Bifidobacterium longum | Synthetic | USA |
| | | infantis Bi-26 | | |
| 21 | Wza | Escherichia coli | Synthetic | USA |
| | | K12 MG1655 | | |
| 22 | LgtA | Neisseria meningitidis | Synthetic | United |
| | | | | Kingdom |
| 23 | WbgO | E. coli O55:H7 | Synthetic | Germany |
| 24 | LgtB | Neisseria meningitidis | Synthetic | United |
| | | MC58 | | Kingdom |
| 25 | glmS*54 | Escherichia coli | Synthetic | USA |
| | | K12 MG1655 | | |
| 26 | galE | Escherichia coli | Synthetic | USA |
| | | K12 MG1655 | | |
| 27 | glmM | Escherichia coli | Synthetic | USA |
| | | K12 MG1655 | | |
| 28 | glmU | Escherichia coli | Synthetic | USA |
| | | K12 MG1655 | | |
| 29 | FutC | Helicobacter pylori | Synthetic | United |
| | | UA1234 | | Kingdom |
| 30 | FucT | Helicobacter pylori | Synthetic | United |
| | | UA1234 | | Kingdom |
| 31 | thyA | Escherichia coli | Synthetic | USA |
| | | K12 MG1655 | | |
| 32 | manA | Escherichia coli | Synthetic | USA |
| | | K12 MG1655 | | |
| 33 | manB | Escherichia coli | Synthetic | USA |
| | | K12 MG1655 | | |
| 34 | manC | Escherichia coli | Synthetic | USA |
| | | K12 MG1655 | | |
| 35 | Gmd | Escherichia coli | Synthetic | USA |
| | | K12 MG1655 | | |
| 36 | fcl | Escherichia coli | Synthetic | USA |
| | | K12 MG1655 | | |
| 37 | fucP | Escherichia coli | Synthetic | USA |
| | | K12 MG1655 | | |
| 38 | Fkp | Bacteroides fragilis | Synthetic | United |
| | | NCTC 9343 | | Kingdom |
| 39 | GNA1 | Saccharomyces | Synthetic | USA |
| | | cerevisiae | | |
| 40 | AGE | Bacteroides ovatus | Synthetic | USA |
| 41 | NeuB | Neisseria meningitidis | Synthetic | United |
| | | | | Kingdom |
| 42 | NeuA | Pasteurella multocida | Synthetic | USA |
| 43 | PmultST3 | Pasteurella multocida | Synthetic | USA |
| 44 | NmeniST3 | Neisseria meningitidis | Synthetic | United |
| | | | | Kingdom |
| 45 | PdST6 | Photobacterium | Synthetic | Japan |
| | | damselae | | |
| 46 | P-JT-ISH- | Photobacterium sp. | Synthetic | Japan |
| | 224-ST6 | JT-ISH-224 | | |
| 47 | yqaB | Escherichia coli | Synthetic | USA |
| | | K12 MG1655 | | |
| 48 | NeuC | Campylobacter jejuni | Synthetic | USA |
| 49 | CscB | Escherichia coli W | Synthetic | USA |
| 50 | Frk | Zymomonas mobilis | Synthetic | United |
| | | | | Kingdom |
| 51 | BaSP | Bifidobacterium | Synthetic | Germany |
| | | adolescentis | | |
| 52 | LacY | Escherichia coli | Synthetic | USA |
| | | K12 MG1655 | | |
| 53 | MdfA | Cronobacter | Synthetic | USA |
| | | sakazakii strain | | |
| | | MOD1_LR753 | | |
| 54 | MdfA | Franconibacter pulveris | Synthetic | Switzerland |
| | | LMG 24059 | | |
| 55 | MdfA | Enterobacter | Synthetic | Germany |
| | | hormaechei | | |
| | | strain 017 | | |
| 56 | MdfA | Citrobacter koseri | Synthetic | Canada |
| | | strain NCTC10771 | | |
| 57 | MdfA | Salmonella enterica | Synthetic | USA |
| | | subsp. Arizonae | | |
| | | serovar 41: z4, z23: | | |
| | | -strain TAMU30EF | | |
| 58 | MdfA | Shigella flexneri | Synthetic | United |
| | | strain 585219 | | Kingdom |
| 59 | MdfA | Yokenella regensburgei | Synthetic | USA |
| | | strain UMB0819 | | |
| 60 | MdfA | Escherichia coli strain | Synthetic | United |
| | | AMC 967 | | Kingdom |
| 61 | MdfA | Klebsiella pneumoniae | Synthetic | USA |
| | | VAKPC309 | | |
| 62 | MdfA | Klebsiella oxytoca strain | Synthetic | United |
| | | 4928STDY7071490 | | Kingdom |
| 63 | MdfA | Klebsiella michiganensis | Synthetic | USA |
| | | strain A2 | | |
| 64 | MdfA | Pluralibacter gergoviae | Synthetic | USA |
| | | strain | | |
| | | FDAARGOS 186 | | |
| 65 | MdfA | Kluyvera ascorbata | Synthetic | USA |
| | | ATCC 33433 | | |
| 66 | MdfA | Enterobacter kobei | Synthetic | Japan |
| 67 | MdfA | Lelliottia sp. WB101 | Synthetic | Germany |
| 68 | MdfA | Citrobacter freundii | Synthetic | Unknown |
| 69 | MdfA | Salmonella enterica | Synthetic | United |
| | | subsp. Salamae | | Kingdom |
| 70 | MdfA | Shigella flexneri | Synthetic | Unknown |
| 71 | MdfA | Cronobacter sakazakii | Synthetic | USA |
| | | strain MOD1 | | |
| | | LR634 | | |
| 72 | MdfA | Cronobacter condimenti | Synthetic | Poland |
| | | strain s37 | | |
| 73 | MdfA | Cronobacter | Synthetic | Canada |
| | | sp. EKM102R | | |
| 74 | MdfA | Cronobacter universalis | Synthetic | United |
| | | NCTC 9529 | | Kingdom |
| 75 | MdfA | Salmonella enterica | Synthetic | USA |
| | | strain 413_SENT | | |
| 76 | MdfA | Klebsiella pneumoniae | Synthetic | Unknown |
| | | subsp. Pneumoniae | | |
| | | strain NCTC11695 | | |
| 77 | MdfA | Klebsiella aerogenes | Synthetic | United |
| | | strain | | Kingdom |
| | | 4928STDY7071344 | | |
| 78 | MdfA | Raoultella planticola | Synthetic | USA |
| | | strain | | |
| | | FDAARGOS_283 | | |
| 79 | MdfA | Klebsiella sp. 2680 | Synthetic | Unkown |
| 80 | MdfA | Kluyvera georgiana | Synthetic | Japan |
| | | strain HRGM_ | | |
| | | Genome_0064 | | |
| 81 | MdfA | Kluyvera intermedia | Synthetic | Unknown |
| | | strain NCTC12125 | | |
| 82 | MdfA | Salmonella enterica | Synthetic | USA |
| | | strain 2014K-0203 | | |
| 83 | MdfA | Salmonella bongori | Synthetic | USA |
| | | strain 85-0051 | | |
| 84 | MdfA | Salmonella enterica | Synthetic | Unknown |
| | | subsp. Enterica | | |
| | | serovar Hillingdon | | |
| | | strain S01-0588 | | |
| 85 | MdfA | Citrobacter youngae | Synthetic | USA |
| | | strain TE1 | | |
| 86 | MdfA | Citrobacter sp. | Synthetic | United |
| | | RHB21-C01 | | Kingdom |
| 87 | MdfA | Citrobacter werkmanii | Synthetic | USA |
| | | strain MGYG- | | |
| | | HGUT-02535 | | |
| 88 | MdfA | Enterobacteriaceae | Synthetic | USA |
| | | bacterium | | |
| | | UBA3109 | | |
| 89 | MdfA | Enterobacteriaceae | Synthetic | USA |
| | | bacterium | | |
| | | UBA6698 | | |
| 90 | MdfA | Leclercia sp. 4-9-1-25 | Synthetic | Germany |
| 91 | MdfA | Lelliottia amnigena | Synthetic | Germany |
| | | strain TZW14 | | |
| 92 | MdfA | Lelliottia aquatilis | Synthetic | Germany |
| | | strain TZW17 | | |
| 93 | MdfA | Enterobacter sp. | Synthetic | United |
| | | RHBSTW-00901 | | Kingdom |
| 94 | MdfA | Enterobacter cloacae | Synthetic | USA |
| | | subsp. Dissolvens | | |
| | | strain GN05902 | | |
| 95 | MdfA | Enterobacter mori | Synthetic | USA |
| | | strain JGM37 | | |
| 96 | MdfA | Kosakonia sacchari | Synthetic | Japan |
| | | strain BO-1 | | |
| 97 | MdfA | Cronobacter malonaticus | Synthetic | USA |
| | | strain | | |
| | | MOD1-Md25g | | |
| 98 | MdfA | Cronobacter | Synthetic | United |
| | | dublinensis 582 | | Kingdom |

TABLE 3
Domain information for membrane proteins of this disclosure

| SEQ | | | | | |
|---|---|---|---|---|---|
| ID | | | | | |
| NO | Family | EggNOG | PFAM | Interpro | TCDB |
| 01- | porters | 07QF7 | PF07690 | IPR011701, | 2.A.1.2 |
| 06 | | | | IPR020846, | |
| | | | | IPR036259, | |
| | | | | IPR005829 | |
| 07 | porters | 05E8G | PF05977 | IPR023722, | 2.A.1.3 |
| | | | | IPR020846, | |
| | | | | IPR036259, | |
| | | | | IPR010290 | |
| 08 | porters | 05JHE | PF07690 | IPR011701, | 2.A.1.1 |
| | | | | IPR020846, | |
| | | | | IPR036259 | |
| 09 | porters | 05EGZ | PF01943 | IPR002797, | 2.A.66 |
| | | | | IPR032896 | |
| 10 | porters | 0814C | PF00893 | IPR000390 | 2.A.7.1 |
| 11 | porters | 07RBJ | PF13347 | IPR039672, | 2.A.2.2 |
| | | | | IPR036259, | |
| | | | | IPR001927 | |
| 12- | porters | 08N8A | PF07690 | IPR011701, | 2.A.1.6 |
| 13 | | | | IPR020846, | |
| | | | | IPR036259 | |
| 14 | porters | 07QRN | PF07690 | IPR011701, | 2.A.1.2 |
| | | | | IPR023721, | |
| | | | | IPR020846, | |
| | | | | IPR036259, | |
| | | | | IPR001411, | |
| | | | | IPR004638 | |
| 15 | P-P-bond- | 08IJ9 | PF00005, | IPR003593, | 3.A.1.1 |
| | hydrolysis- | | PF17912 | IPR003439, | |
| | driven | | | IPR017871, | |
| | transporters | | | IPR008995, | |
| | | | | IPR040582, | |
| | | | | IPR027417 | |
| 16 | P-P-bond- | 05CJ1 | PF00005 | IPR003593, | 3.A.1.1 |
| | hydrolysis- | | | IPR003439, | |
| | driven | | | IPR017871, | |
| | transporters | | | IPR015851, | |
| | | | | IPR005978, | |
| | | | | IPR027417 | |
| 17 | P-P-bond- | 05DFW | PF13407 | IPR013456, | 3.A.1.2 |
| | hydrolysis- | | | IPR025997, | |
| | driven | | | IPR028082 | |
| | transporters | | | | |
| 18 | P-P-bond- | 05EZD | PF00528 | IPR000515, | 3.A.1.1 |
| | hydrolysis- | | | IPR035906 | |
| | driven | | | | |
| | transporters | | | | |
| 19 | P-P-bond- | 05I1K | PF00528 | IPR000515, | 3.A.1.1 |
| | hydrolysis- | | | IPR035906 | |
| | driven | | | | |
| | transporters | | | | |
| 20 | P-P-bond- | 07HR3 | PF00528 | IPR000515, | 3.A.1.1 |
| | hydrolysis- | | | IPR035906 | |
| | driven | | | | |
| | transporters | | | | |
| 21 | B-Barrel | 05DAY | PF02563, | IPR003715, | 1.B.18 |
| | Porins | | PF10531, | IPR019554, | |
| | | | PF18412 | IPR040716 | |
| 53- | porters | 07QF7 | PF07690 | IPR011701, | 2.A.1.2 |
| 98 | | | | IPR020846, | |
| | | | | IPR036259, | |
| | | | | IPR005829 | |

Cultivation Conditions

A preculture of 96-well microtiter plate experiments was started from a cryovial, in 150 μL LB and was incubated overnight at 37° C. on an orbital shaker at 800 rpm. This culture was used as inoculum for a 96well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations). Also, a dilution of the cultures was made to measure the optical density at 600 nm. The cell performance index or CPI was determined by dividing the concentrations of the oligosaccharide with LN3 as a core trisaccharide measured in the whole broth, unless specified otherwise, by the biomass, in relative percentages compared to the reference strain. The biomass is empirically determined to be approximately ⅓^{rd} of the optical density measured at 600 nm. The export ratio of the oligosaccharide with LN3 as a core trisaccharide was determined by dividing the concentrations of the oligosaccharide with LN3 as a core trisaccharide measured in the supernatant by the concentrations of the oligosaccharide with LN3 as a core trisaccharide measured in the whole broth, in relative percentages compared to the reference strain.

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H2SO4 and 20% NH40H. The exhaust gas was cooled. 10% solution of silicone antifoaming agent was added when foaming raised during the fermentation.

Optical Density

Cell density of the cultures was frequently monitored by measuring optical density at 600 nm (Implen Nanophotometer NP80, Westburg, Belgium or with a Spark 10M microplate reader, Tecan, Switzerland).

Productivity

The specific productivity Qp is the specific production rate of the product (which is the oligosaccharide with LN3 as a core trisaccharide), typically expressed in mass units of product per mass unit of biomass per time unit (=g oligosaccharide/g biomass/h). The Qp value has been determined for each phase of the fermentation runs, i.e., Batch and Fed-Batch phase, by measuring both the amount of product and biomass formed at the end of each phase and the time frame each phase lasted.

The specific productivity Qs is the specific consumption rate of the substrate, e.g., sucrose, typically expressed in mass units of substrate per mass unit of biomass per time unit (=g sucrose/g biomass/h). The Qs value has been determined for each phase of the fermentation runs, i.e., Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g oligosaccharide/g sucrose). The Ys has been determined for each phase of the fermentation runs, i.e., Batch and Fed-Batch phase, by measuring both the total amount of oligosaccharide with LN3 as a core trisaccharide produced and total amount of sucrose consumed at the end of each phase.

The yield on biomass Yx is the fraction of biomass that is made from substrate and is typically expressed in mass unit of biomass per mass unit of substrate (=g biomass/g sucrose). The Yp has been determined for each phase of the fermentation runs, i.e., Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g oligosaccharide/L/h). The rate is determined by measuring the concentration of the oligosaccharide with LN3 as a core trisaccharide that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed/h). The lactose conversion rate is determined by measurement of the total lactose that is consumed during a fermentation run, divided by the total fermentation time.

Growth Rate/Speed Measurement

The maximal growth rate (μMax) was calculated based on the observed optical densities at 600 nm using the R package grofit.

Analytical Analysis

Standards such as but not limited to sucrose, lactose, lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

Normalization of the Data

For all types of cultivation conditions, data obtained from the mutant strains was normalized against data obtained in identical cultivation conditions with reference strains having an identical genetic background as the mutant strains but lacking the membrane protein expression cassettes (i.e., setpoint). All data (including the data in Tables 4-11) is hence given in relative percentages to that setpoint.

### Example 5. Membrane Proteins Tested for LN3 Production in an E. coli Host Cultivated 72 h in a Growth Experiment in Minimal Media Supplemented with 20 g/L Lactose

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production of a host cell growing in minimal media supplemented with 20 g/L lactose. Candidate genes were presented to the LN3 production hosts on a pSC101 plasmid. A growth experiment was performed according to the cultivation conditions provided in Example 4. The production of LN3 was evaluated in each LN3 production host expressing the galactoside beta-1,3-N-acetylglucosaminyltransferase (LgtA) from N. meningitidis with SEQ ID NO: 22.

### Example 6. Membrane Proteins Identified that Enhance LN3 and Lacto-N-Tetraose (LNT) Production in an E. coli Host Cultivated 72 h in a Growth Experiment in Minimal Media Supplemented with 20 g/L Lactose

An experiment was set up to evaluate membrane proteins with SEQ ID NOs: 01, 03, 07, 08, 09, 10, 11, 12, 13, 14, 15, 16, 17, 18, 19 and 21 for their ability to enhance LN3 and lacto-N-tetraose (LNT) production of a host cell growing in minimal media supplemented with 20 g/L lactose. Candidate genes were presented to the LNT production hosts on a pSC101 plasmid. A growth experiment was performed according to the cultivation conditions provided in Example 4.

Table 4 present whole broth measurements of LN3 and of LNT for the different strains expressing a membrane protein compared to a reference strain without membrane protein expressed. According to Table 4, expression of a membrane protein with SEQ ID NO: 01, 03, 07, 08, 09, 10, 11, 12, 13, 14, 15, 16, 17, 18, 19 or 21 enhanced the production of LN3 as well as of LNT that is being produced in a LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (WbgO) from E. coli O55:H7 with SEQ ID NO: 23.

TABLE 4

| | % LN3 production | | % LNT production | |
|---|---|---|---|---|
| | (whole broth) | | (whole broth) | |
| SEQ | compared to | | compared to | |
| ID | reference strain | SD | reference strain | SD |
| 01 | 798.1 | ±36 | 574 | ±20 |
| 03 | 483.1 | ±140 | 157 | ±90 |
| 07 | 155.4 | ±26 | 132 | ±2 |
| 08 | 150.6 | ±29 | 123 | ±12 |
| 09 | 141.3 | ±11 | 111 | ±14 |
| 10 | 182.7 | ±27 | 150 | ±14 |
| 11 | 200.8 | ±6 | 147 | ±4 |
| 12 | 116.6 | ±13 | 111 | ±11 |
| 13 | 151.9 | ±0.6 | 118 | ±5 |
| 14 | 190.4 | ±59 | 132 | ±32 |
| 15 | 161.5 | ±2.8 | 140 | ±2 |
| 16 | 204.5 | ±16 | 173 | ±9 |
| 17 | 166.8 | ±75 | 165 | ±137 |
| 18 | 185.6 | ±43 | 129 | ±19 |
| 19 | 159.5 | ±2.4 | 140 | ±19 |
| 21 | 125.2 | ±22 | 111 | ±8 |

“SD” represents the standard deviation (4 replicates of the same strain tested). The “reference strain” is identical to the tested strains, except that the indicated membrane protein (SEQ ID NO indicated) is not expressed in the reference strain.

Further, Table 5 shows that the cell performance index (CPI) for the different strains expressing a membrane protein as outlined in present example is higher compared to a reference strain without the membrane protein expressed.

TABLE 5

| | % CPI (g LN3/g | | % CPI (g LNT/g | |
|---|---|---|---|---|
| | biomass), in whole | | biomass), in whole | |
| SEQ | broth, compared | | broth, compared | |
| ID | to reference strain | SD | to reference strain | SD |
| 01 | 427 | ±62 | 418 | ±47 |
| 03 | 335 | ±51 | 141 | ±54 |
| 07 | 110 | ±19 | 128 | ±1 |
| 08 | 110 | ±11 | 123 | ±1 |
| 09 | 112 | ±4 | 121 | ±10 |
| 10 | 128 | ±16 | 143 | ±11 |
| 11 | 144 | ±4 | 143 | ±3 |
| 12 | 96 | ±9 | 116 | ±9 |
| 13 | 117 | ±2 | 124 | ±3 |
| 14 | 126 | ±25 | 118 | ±16 |
| 15 | 113 | ±2 | 134 | ±1 |
| 16 | 115 | ±10 | 133 | ±6 |
| 17 | 109 | ±50 | 122 | ±78 |
| 18 | 128 | ±3 | 121 | ±21 |
| 19 | 105 | ±13 | 125 | ±1 |
| 21 | 108 | ±7 | 118 | ±6 |

“SD” represents the standard deviation (4 replicates of the same strain tested). The “reference strain” is identical to the tested strains, except that the indicated membrane protein (SEQ ID NO indicated) is not expressed in the reference strain.

### Example 7. Membrane Proteins Identified that Enhance Lacto-N-Tetraose (LNT) Production in an E. coli Host Cultivated 72 h in a Growth Experiment in Minimal Media Supplemented with 20 g/L Lactose

An experiment was set up to evaluate membrane proteins with SEQ ID NOs: 02, 04, 05, 66, 67 and 68 for their ability to enhance lacto-N-tetraose (LNT) production of a host cell growing in minimal media supplemented with 20 g/L lactose. Candidate genes were presented to the LNT production hosts on a pSC101 plasmid. A growth experiment was performed according to the cultivation conditions provided in Example 4. The strains in the present example are hence the same as in Example 6 except for the membrane protein that is evaluated.

Experimental data from these 6 different membrane proteins described above is depicted in Table 6.

All 6 strains are able to produce more LNT and show a higher cell performance index (CPI) compared to the reference strain (without membrane protein expressed) in whole broth samples when evaluated in a growth experiment according to the culture conditions provided in Example 4, in which the culture medium contains sucrose as carbon source and lactose as precursor. Moreover, these strains did not show a higher production of LN3 (% LN3 production compared to reference strain, measured in whole broth) in contrast to the strains used in Example 6. The strains in present Example 7 are hence particularly advantageous for the production of LNT wherein the fraction of LN3 should be kept low as possible, whereas the strains in Example 6 are useful if a mixture of LN3 and LNT is aimed for or when the relative amount of LN3 in the produced oligosaccharide fraction is not important.

The LNT production host used for this screening expressed the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

TABLE 6

| | % LNT production | | % CPI (g LNT/g | |
|---|---|---|---|---|
| | (whole broth) | | biomass), in whole | |
| SEQ | compared to | | broth, compared to | |
| ID | reference strain | SD | reference strain | SD |
| 02 | 117 | ±4 | 169 | ±39 |
| 04 | 137 | ±8 | 151 | ±26 |
| 05 | 197 | ±8 | 181 | ±49 |
| 66 | 117 | ±4 | 147 | ±26 |
| 67 | 122 | ±9 | 117 | ±15 |
| 68 | 114 | ±4 | 142 | ±22 |

“SD” represents the standard deviation (4 replicates of the same strain tested). The “reference strain” is identical to the tested strains, except that the indicated membrane protein (SEQ ID NO indicated) is not expressed in the reference strain.

### Example 8. Membrane Proteins Identified that Enhance Lacto-N-Neotetraose (LNnT) Production in an E. coli Host Cultivated 72 h in a Growth Experiment in Minimal Media Supplemented with 20 g/L Lactose

An experiment was set up to evaluate membrane proteins with SEQ ID NOs: 01, 02, 03, 05, 66 and 68 for their ability to enhance LN3 and/or lacto-N-neotetraose (LNnT) production of a host cell growing in minimal media supplemented with 20 g/L lactose. Candidate genes were presented to the LNnT production hosts on a pSC101 plasmid. A growth experiment was performed according to the cultivation conditions provided in Example 4.

Experimental data from these 6 different membrane proteins described above is depicted in Table 7.

All 6 strains are able to produce more LNnT compared to the reference strain (without membrane protein expressed) in whole broth samples when evaluated in a growth experiment according to the culture conditions provided in Example 4, in which the culture medium contains sucrose as carbon source and lactose as precursor. The LNnT production host used for this screening expressed the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

TABLE 7

| SEQ | % LNnT production (whole broth) | |
|---|---|---|
| ID | compared to reference strain | SD |
| 01 | 240 | ±3 |
| 02 | 266 | ±36 |
| 03 | 222 | ±19 |
| 05 | 139 | ±19 |
| 66 | 143 | ±5 |
| 68 | 217 | ±34 |

“SD” represents the standard deviation (4 replicates of the same strain tested). The “reference strain” is identical to the tested strains, except that the indicated membrane protein (SEQ ID NO indicated) is not expressed in the reference strain.

### Example 9. Evaluation of Membrane Proteins in E. coli LN3 Production Hosts in 5 L Fermentation Runs

An LN3 production host described in Example 4 was modified to express the membrane protein with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LN3 production host but lacking the membrane protein gene was analyzed in identical fermentation settings. At the end of fermentation, the titers measured in supernatant and whole broth samples varies between 80 g/L and 125 g/L for the strains expressing membrane protein genes. The reference strain reaches LN3 titers between 45 g/L and 65 g/L measured in supernatant and whole broth samples, which shows the positive effect of the membrane proteins with SEQ ID 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LN3 production in 5 L fermentation runs.

### Example 10. Evaluation of Membrane Proteins in E. coli LN3 Production Hosts in 5 L Fermentation Runs

An LN3 production host described in Example 4 was modified to express the membrane protein with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LN3 production host but lacking the membrane protein gene was analyzed in identical fermentation settings. At the end of fermentation, the LN3 production in the modified strains was 125% to 250% compared to the reference strain, supporting the positive effect of the membrane proteins with SEQ ID 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LN3 production in 5 L fermentation runs.

### Example 11. Evaluation of Membrane Proteins in E. coli LNT Production Hosts in 5 L Fermentation Runs

An LNT production host described in Example 4 was modified to express the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LNT production host but lacking the membrane protein gene was analyzed in identical fermentation settings. At the end of fermentation, the titers measured in supernatant and whole broth samples varies between 90 g/L and 130 g/L for the strains expressing membrane protein genes. The reference strain reaches LNT titers between 50 g/L and 70 g/L measured in supernatant and whole broth samples, which shows the positive effect of the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LNT production in 5 L fermentation runs.

### Example 12. Evaluation of Membrane Proteins in E. coli LNT Production Hosts in 5 L Fermentation Runs

An LNT production host described in Example 4 was modified to express the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LNT production host but lacking the membrane protein gene was analyzed in identical fermentation settings. At the end of fermentation, the LNT production in the modified strains was 135% to 265% compared to the reference strain, supporting the positive effect of the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LNT production in 5 L fermentation runs.

### Example 13. Evaluation of Membrane Proteins in E. coli LNT Production Hosts in 5 L Fermentation Runs

An LNT production host described in Example 4 was modified to express the membrane proteins with SEQ ID NO: 01 from pSC101 plasmid. Modified strains were evaluated for their productivity in bioreactor settings (5 L fermenter). Fermentation runs were performed according to the conditions provided in Example 4. Also, a reference strain identical to the LNT production host but lacking the membrane protein gene was analyzed in identical fermentation settings. Experimental data from the fermentations is depicted in Tables 8 and 9.

At the end of fermentation, the titers measured in the supernatant and whole broth samples were higher than what was measured in the reference strain (Table 8). It was further observed that LNT secretion was enhanced compared to the reference strain, as the increase in LNT production compared to the reference strain is higher for the supernatants than for the whole broth. Moreover, the production rate of LNT, the lactose consumption rate and the LNT yield on sucrose were higher compared to the reference strain (Table 9). Altogether, these data show the positive effects of the membrane protein with SEQ ID NO: 01 on LNT production in 5 L fermentation runs.

TABLE 8

| | % LNT production | % LNT production |
|---|---|---|
| | (supernatans) | (whole broth) |
| SEQ | compared to | compared to |
| ID | reference strain | reference strain |
| 01 | 220 | 147 |

The “reference strain” is identical to the tested strains, except that the indicated membrane protein (SEQ ID NO indicated) is not expressed in the reference strain.

TABLE 9

| | | | % lactose |
|---|---|---|---|
| | % production rate | % yield on sucrose | consumption |
| | (g LNT/L/h), | (g LNT/g sucrose), | rate (g lactose/h), |
| | in whole broth, | in whole broth, | in whole broth, |
| SEQ | compared to | compared to | compared to |
| ID | reference strain | reference strain | reference strain |
| 01 | 151 | 150 | 136 |
| | | | % lactose |
| | % production rate | % yield on sucrose | consumption |
| | (g LNT/L/h), | (g LNT/g sucrose), | rate (g lactose/h), |
| | in supernatans, | in supernatans, | in supernatans, |
| SEQ | compared to | compared to | compared to |
| ID | reference strain | reference strain | reference strain |
| 01 | 230 | 235 | 187 |

The “reference strain” is identical to the tested strains, except that the indicated membrane protein (SEQ ID NO indicated) is not expressed in the reference strain.

### Example 14. Evaluation of Membrane Proteins in E. coli LNnT Production Hosts in 5 L Fermentation Runs

An LNnT production host described in Example 4 was modified to express the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LNnT production host but lacking the membrane protein gene was analyzed in identical fermentation settings. At the end of fermentation, the titers measured in supernatant and whole broth samples varies between 70 g/L and 100 g/L for the strains expressing membrane protein genes. The reference strain reaches LNnT titers between 40 g/L and 60 g/L measured in supernatant and whole broth samples, which shows the positive effect of the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LNnT production in 5 L fermentation runs.

### Example 15. Evaluation of Membrane Proteins in E. coli LNnT Production Hosts in 5 L Fermentation Runs

An LNnT production host described in Example 4 was modified to express the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 from pSC101 plasmid. Modified strains were evaluated for their productivity in bioreactor settings (5 L fermenter). Fermentation runs were performed according to the conditions provided in Example 4. Also, a reference strain identical to the LNnT production host but lacking the membrane protein gene was analyzed in identical fermentation settings. At the end of fermentation, the LNnT production in the modified strains was 120% to 245% compared to the reference strain, supporting the positive effect of the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 on LNnT production in 5 L fermentation runs.

### Example 16. Evaluation of Membrane Proteins in E. coli LNnT Production Hosts in 5 L Fermentation Runs

An LNnT production host described in Example 4 was modified to express the membrane protein with SEQ ID NO: 02 from pSC101 plasmid. Modified strain was evaluated for its productivity in bioreactor settings (5 L fermenter). Fermentation runs were performed according to the conditions provided in Example 4. Also, a reference strain identical to the LNnT production host but lacking the membrane protein gene was analyzed in identical fermentation settings. Experimental data from the fermentations are depicted in Tables 10 and 11.

At the end of fermentation, the titers measured in the supernatant and whole broth samples were higher than what was measured in the reference strain (Table 10). Moreover, a higher cell performance index (CPI) is obtained compared to the reference strain. It was further observed that LNnT secretion was enhanced compared to the reference strain, as the increase in LNnT production compared to the reference strain is higher for the supernatants than for the whole broth. Moreover, the production rate of LNnT, the lactose consumption rate, the LNnT yield on sucrose and the specific productivity of LNnT were higher compared to the reference strain (Table 11). Altogether, these data show the positive effects of the membrane protein with SEQ ID NO: 02 on LNnT production in 5 L fermentation runs.

TABLE 10

| | | % CPI | | % CPI |
|---|---|---|---|---|
| | % LNnT | (g LNnT/g | % LNnT | (g LNnT/g |
| | production | Biomass), in | production | Biomass), in |
| | (supernatans) | supernatans, | (whole broth) | whole broth, |
| | compared to | compared | compared to | compared |
| SEQ | reference | to reference | reference | to reference |
| ID | strain | strain | strain | strain |
| 02 | 160 | 183 | 127 | 136 |

The “reference strain” is identical to the tested strains, except that the indicated membrane protein (SEQ ID NO indicated) is not expressed in the reference strain.

TABLE 11

| | % | % yield on | % lactose | % specific |
|---|---|---|---|---|
| | production | sucrose | consumption | productivity |
| | rate | (g LNnT/g | rate (g | (g LNnT/g |
| | (g LNnT/ | sucrose), | lactose/h), | biomass/ |
| | L/h), in | in whole | in whole | h), in |
| | whole broth, | broth, | broth, | whole broth, |
| | compared to | compared | compared | compared |
| SEQ | reference | to reference | to reference | to reference |
| ID | strain | strain | strain | strain |
| 02 | 130 | 117 | 97 | 139 |
| | % | | % lactose | % specific |
| | production | % yield on | consumption | productivity |
| | rate | sucrose | rate (g | (g LNnT/g |
| | (g LNnT/ | (g LNnT/g | lactose/h), | biomass/h), |
| | L/h), in | sucrose), in | in | in |
| | supernatans, | supernatans, | supernatans, | supernatans, |
| | compared to | compared | compared | compared |
| SEQ | reference | to reference | to reference | to reference |
| ID | strain | strain | strain | strain |
| 02 | 165 | 186 | 107 | 177 |

The “reference strain” is identical to the tested strains, except that the indicated membrane protein (SEQ ID NO indicated) is not expressed in the reference strain.

### Example 17. Materials and Methods Saccharomyces cerevisiae

Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

Plasmids

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g., galE from E. coli (UniProt ID P09147). This plasmid can be further modified with constitutive transcriptional units for a lactose permease like e.g., LAC12 from K. lactis (UniProt ID P07921) and a galactoside beta-1,3-N-acetylglucosaminyltransferase activity like e.g., lgtA from N. meningitidis (SEQ ID NO: 22) to produce LN3 (lacto-N-triose, LNT-II). To further produce LN3-derived oligosaccharides like LNT, the mutant LN3 producing strains were further modified with a constitutive transcriptional unit for an N-acetylglucosamine beta-1,3-galactosyltransferase like e.g., WbgO from E. coli O55:H7 (SEQ ID NO: 23).

In an example for production of LN3 derived oligosaccharides like lacto-N-neotetraose (LNnT, Gal-b1,4-GlcNAc-b1,3-Gal-b1,4-Glc), the mutant LN3 producing strain were further modified with a constitutive transcriptional unit for an N-acetylglucosamine beta-1,4-galactosyltransferase like e.g., LgtB from Neisseria meningitidis (SEQ ID NO: 24).

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast on and the Ura3 selection marker for selection and maintenance in yeast. This plasmid is further modified with constitutive transcriptional units for a lactose permease like e.g., LAC12 from K. lactis (UniProt ID P07921), a GDP-mannose 4,6-dehydratase like e.g., gmd from E. coli (SEQ ID NO: 35) and a GDP-L-fucose synthase like e.g., fcl from E. coli (SEQ ID NO: 36). The yeast expression plasmid p2a_2μ_Fuc2 can be used as an alternative expression plasmid of the p2a_2μ_Fuc plasmid comprising next to the ampicillin resistance gene, the bacterial ori, the 2μ yeast on and the Ura3 selection, constitutive transcriptional units for a lactose permease like e.g., LAC12 from K. lactis (UniProt ID P07921), a fucose permease like e.g., fucP from E. coli (SEQ ID NO: 37) and a bifunctional enzyme with fucose kinase/fucose-1-phosphate guanylyltransferase activity like e.g., fkp from Bacteroides fragilis (SEQ ID NO: 38). To further produce fucosylated oligosaccharides with LN3 as a core trisaccharide, the p2a_2μ_Fuc and its variant the p2a_2μ_Fuc2, additionally contained a constitutive transcriptional unit for the H. pylori alpha-1,2-fucosyltransferase with SEQ ID NO: 29 (HpFutC) and/or the H. pylori alpha-1,3-fucosyltransferase with SEQ ID NO: 30 (HpFucT).

To produce sialic acid and CMP-sialic acid, a yeast expression plasmid was derived from the pRS420-plasmid series (Christianson et al., 1992, Gene 110: 119-122) containing the TRP1 selection marker and constitutive transcriptional units for the mutant glmS*54 from E. coli with SEQ ID NO: 25, the phosphatase yqaB from E. coli with SEQ ID NO: 47, the N-acetylglucosamine 2-epimerase (AGE) from B. ovatus with SEQ ID NO: 40, the N-acetylneuraminate synthase (NeuB) from N. meningitidis with SEQ ID NO: 41 and the N-acylneuraminate cytidylyltransferase (NeuA) from P. multocida with SEQ ID NO: 42. Optionally, a constitutive transcriptional unit for GNA1 from S. cerevisiae with SEQ ID NO: 39 was added as well.

For production of sialylated oligosaccharides with LN3 as a core trisaccharide the plasmid further comprised constitutive transcriptional units for the lactose permease (LAC12) from K. lactis (UniProt ID P07921), and one or more sialyltransferase(s) comprising of a beta-galactoside alpha-2,3-sialyltransferase comprising SEQ ID NO: 43 (PmultST3) from P. multocida and/or SEQ ID NO: 44 (NmeniST3) from N. meningitidis, and/or a beta-galactoside alpha-2,6-sialyltransferase comprising SEQ ID NO: 45 (PdST6) from Photobacterium damselae and/or SEQ ID NO: 46 (P-JT-ISH-224-ST6) from Photobacterium sp. JT-ISH-224.

Preferably, but not necessarily, any one or more of the glycosyltransferase and/or the proteins involved in nucleotide-activated sugar synthesis were N- and/or C-terminally fused to a SUMOstar tag (e.g., obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g., Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275). Plasmids were maintained in the host E. coli DH5alpha (F⁻, phi80d1acZdeltaM15, delta(1acZYA-argF)U169, deoR, recA1, endA1, hsdR17(rk⁻, mk⁺), phoA, supE44, lambda-, thi-1, gyrA96, relA1) bought from Invitrogen.

In a next step, the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 as enlisted in Tables 2 and 3 can be evaluated in above mutant strains. Membrane protein genes can be evaluated either present on a p2a 2μ_Fuc or p2a_2μ_Fuc2 or pRS420 (Christianson et al., 1992, Gene 110: 119-122; having a HIS3 selection marker) plasmid or integrated in the host's genome in constitutive transcriptional units.

Genes were expressed using synthetic constitutive promoters, as described by Blazeck (Biotechnology and Bioengineering, Vol. 109, No. 11, 2012).

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

Cultivation Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

Analytical Analysis

It is referred to this section of Example 4.

### Example 18. Membrane Proteins Tested for LN3 Production in a S. cerevisiae Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production of a host cell growing in media supplemented with 20 g/L lactose as described in Example 17. Candidate genes were presented to the LN3 production hosts on a pRS420 plasmid. A growth experiment was performed according to the cultivation conditions provided in Example 17. The production of LN3 was evaluated in each LN3 production host expressing the galactoside beta-1,3-N-acetylglucosaminyltransferase (LgtA) from N. meningitidis with SEQ ID NO: 22.

### Example 19. Membrane Proteins Tested for Lacto-N-Tetraose (LNT) Production in a S. cerevisiae Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-tetraose (LNT) production of a host cell growing in media supplemented with 20 g/L lactose as described in Example 17. Candidate genes were presented to the LNT production hosts on a pRS420 plasmid. A growth experiment was performed according to the cultivation conditions provided in Example 17. The production of LN3 and LNT was evaluated in each LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

### Example 20. Membrane Proteins Identified that Enhance Lacto-N-Neotetraose (LNnT) Production in a S. cerevisiae Host

An experiment was set up to evaluate membrane proteins with SEQ ID NOs SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-neotetraose (LNnT) production of a host cell growing in media supplemented with 20 g/L lactose as described in Example 17. Candidate genes were presented to the LNnT production hosts on a pRS420 plasmid. A growth experiment was performed according to the cultivation conditions provided in Example 17. The production of LN3 and LNnT was evaluated in each LNnT production host expressing the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

### Example 21. Materials and Methods Chlamydomonas reinhardtii Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na2EDTA·H2O (Titriplex III), 22 g/L ZnSO4·7H2O, 11.4 g/L H3BO3, 5 g/L MnCl2·4H2O, 5 g/L FeSO4·7H2O, 1.6 g/L CoCl2·6H2O, 1.6 g/L CuSO4·5H2O and 1.1 g/L (NH4)6MoO3.

The TAP medium contained 2.42 g/L Tris (tris(hydroxymethyl)aminomethane), 25 mg/L salt stock solution, 0.108 g/L K2HPO4, 0.054 g/L KH2PO4 and 1.0 mL/L glacial acetic acid. The salt stock solution consisted of 15 g/L NH4CL, 4 g/L MgSO4·7H2O and 2 g/L CaCl2·2H2O. As precursor for saccharide synthesis, precursors like e.g., lactose (e.g., 20 g/L), galactose, glucose, fructose, fucose, GlcNAc could be added. Medium was sterilized by autoclaving (121° C., 21′). For stock cultures on agar slants TAP medium was used containing 1% agar (of purified high strength, 1000 g/cm²).

Strains

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (www.chlamycollection.org), University of Minnesota, U.S.A.

Plasmids

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×107 cells/mL. Then, the cells were inoculated into fresh liquid TAP medium in a concentration of 1.0×106 cells/mL and grown under continuous light for 18-20 h until the cell density reached 4.0×106 cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×107 cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575Ω, 50 μFD). After electroporation, the cuvette was immediately placed on ice for 10 min. Finally, the cell suspension was transferred into a 50 ml conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+−0.5° C. under continuous illumination with a light intensity of 8000 Lx. Cells were analyzed 5-7 days later.

In an example to produce UDP-galactose, C. reinhardtii cells are modified with transcriptional units comprising the genes encoding a galactokinase like e.g., from Arabidopsis thaliana (KIN, UniProt ID Q9SEE5) and an UDP-sugar pyrophosphorylase like e.g., USP from A. thaliana (UniProt ID Q9C5I1).

In an example to produce LN3, a constitutive transcriptional comprising a galactoside beta-1,3-N-acetylglucosaminyltransferase like e.g., lgtA from N. meningitidis (SEQ ID NO: 22). In an example for LNT production, the LN3 producing strain is further modified with a constitutive transcriptional unit comprising an N-acetylglucosamine beta-1,3-galactosyltransferase like e.g., WbgO from E. coli O55:H7 (SEQ ID NO: 23). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO: 24).

To produce GDP-fucose, C. reinhardtii cells are modified with a transcriptional unit for a GDP-fucose synthase like e.g., from Arabidopsis thaliana (GER1, UniProt ID 049213).

To further produce fucosylated oligosaccharides with LN3 as a core trisaccharide, C. reinhardtii cells can be modified with an expression plasmid comprising a constitutive transcriptional unit for an alpha-1,2-fucosyltransferase like e.g., HpFutC from H. pylori (SEQ ID NO: 29) and/or an alpha-1,3-fucosyltransferase like e.g., HpFucT from H. pylori (SEQ ID NO: 30).

In a next step, the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 as enlisted in Tables 2 and 3 can be evaluated in above mutant strains. Membrane protein genes can be evaluated either present on a suitable expression plasmid or integrated in the host's genome in constitutive transcriptional units.

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation.

For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

Analytical Analysis

It is referred to this section of Example 4.

### Example 22. Membrane Proteins Tested for LN3 Production in a C. reinhardtii Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production of a host cell growing in media (see Example 21) supplemented with 20 g/L lactose. Candidate genes were presented to the LN3 production hosts by genomic knock-in of a constitutive transcriptional unit comprising the membrane protein gene. A growth experiment was performed according to the cultivation conditions provided in Example 21. The production of LN3 was evaluated in each LN3 production host expressing the galactoside beta-1,3-N-acetylglucosaminyltransferase (LgtA) from N. meningitidis with SEQ ID NO: 22.

### Example 23. Membrane Proteins Tested for Lacto-N-Tetraose (LNT) Production in a C. reinhardtii Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-tetraose (LNT) production of a host cell growing in media (see Example 21) supplemented with 20 g/L lactose. Candidate genes were presented to the LNT production hosts by genomic knock-in of a constitutive transcriptional unit comprising the membrane protein gene. A growth experiment was performed according to the cultivation conditions provided in Example 21. The production of LN3 and LNT was evaluated in each LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

### Example 24. Membrane Proteins Identified that Enhance Lacto-N-Neotetraose (LNnT) Production in a C. reinhardtii Host

An experiment was set up to evaluate membrane proteins with SEQ ID NOs SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-neotetraose (LNnT) production of a host cell growing in media (see Example 21) supplemented with 20 g/L lactose. Candidate genes were presented to the LNnT production hosts by genomic knock-in of a constitutive transcriptional unit comprising the membrane protein gene. A growth experiment was performed according to the cultivation conditions provided in Example 21. The production of LN3 and LNnT was evaluated in each LNnT production host expressing the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

### Example 25. Materials and Methods Bacillus subtilis Media

Two different media are used, namely a rich Luria Broth (LB) and a minimal medium for shake flask (MMsf). The minimal medium uses a trace element mix.

Trace element mix consisted of 0.735 g/L CaCl2·2H2O, 0.1 g/L MnCl2·2H2O, 0.033 g/L CuCl2·2H2O, 0.06 g/L CoCl2·6H2O, 0.17 g/L ZnCl2, 0.0311 g/L H3B04, 0.4 g/L Na2EDTA·2H2O and 0.06 g/L Na2MoO4. The Fe-citrate solution contained 0.135 g/L FeCl3·6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH4)2SO4, 7.5 g/L KH2PO4, 17.5 g/L K2HPO4, 1.25 g/L Na-citrate, 0.25 g/L MgSO4·7H2O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment, lactose (20 g/L) was added.

Complex medium, e.g., LB, was sterilized by autoclaving (121° C., 21′) and minimal medium by filtration (0.22 μm Sartorius). When necessary, the medium was made selective by adding an antibiotic (e.g., zeocin (20 mg/L)).

Strain

Bacillus subtilis 168, available at Bacillus Genetic Stock Center (Ohio, USA).

Plasmids

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation. In an example to produce LN3, Bacillus subtilis mutant strains are created to contain a gene coding for a lactose importer (such as the E. coli lacY with SEQ ID NO: 52) and further contain a constitutive transcriptional unit comprising a galactoside beta-1,3-N-acetylglucosaminyltransferase like e.g., lgtA from N. meningitidis (SEQ ID NO: 22). In an example for LNT production, the LN3 producing strain is further modified with a constitutive transcriptional unit comprising an N-acetylglucosamine beta-1,3-galactosyltransferase like e.g., WbgO from E. coli O55:H7 (SEQ ID NO: 23). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO: 24). To further produce fucosylated oligosaccharides with LN3 as a core trisaccharide, B. subtilis cells can be modified with an expression plasmid (or via a genomic knock-in) comprising a constitutive transcriptional unit for an alpha-1,2-fucosyltransferase like e.g., HpFutC from H. pylori (SEQ ID NO: 29) and/or an alpha-1,3-fucosyltransferase like e.g., HpFucT from H. pylori (SEQ ID NO: 30).

In a next step, the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 as enlisted in Tables 2 and 3 can be evaluated in above mutant strains. Membrane protein genes can be evaluated either present on a suitable expression plasmid as described herein or integrated in the host's genome in constitutive transcriptional units.

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer.

Analytical Analysis

It is referred to this section of Example 4.

### Example 26. Membrane Proteins Tested for LN3 Production in a B. subtilis Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production of a host cell growing in media (see Example 25) supplemented with 20 g/L lactose. Candidate genes were presented to the LN3 production hosts by genomic knock-in of a constitutive transcriptional unit comprising the membrane protein gene. A growth experiment was performed according to the cultivation conditions provided in Example 25. The production of LN3 was evaluated in each LN3 production host expressing the galactoside beta-1,3-N-acetylglucosaminyltransferase (LgtA) from N. meningitidis with SEQ ID NO: 22.

### Example 27. Membrane Proteins Tested for Lacto-N-Tetraose (LNT) Production in a B. subtilis Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-tetraose (LNT) production of a host cell growing in media (see Example 25) supplemented with 20 g/L lactose. Candidate genes were presented to the LNT production hosts by genomic knock-in of a constitutive transcriptional unit comprising the membrane protein gene. A growth experiment was performed according to the cultivation conditions provided in Example 25. The production of LN3 and LNT was evaluated in each LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

### Example 28. Membrane Proteins Identified that Enhance Lacto-N-Neotetraose (LNnT) Production in a B. subtilis Host

An experiment was set up to evaluate membrane proteins with SEQ ID NOs SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-neotetraose (LNnT) production of a host cell growing in media (see Example 25) supplemented with 20 g/L lactose. Candidate genes were presented to the LNnT production hosts by genomic knock-in of a constitutive transcriptional unit comprising the membrane protein gene. A growth experiment was performed according to the cultivation conditions provided in Example 25. The production of LN3 and LNnT was evaluated in each LNnT production host expressing the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

### Example 29. Materials and Methods Corynebacterium glutamicum

Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl2), 10 g/L FeSO4·7H2O, 10 g/L MnSO4·H2O, 1 g/L ZnSO4·7H2O, 0.2 g/L CuSO4, 0.02 g/L NiCl2·6H2O, 0.2 g/L biotin (pH 7.0) and 0.03 g/L protocatechuic acid.

The minimal medium for the shake flasks (MMsf) experiments contained 20 g/L (NH4)2SO4, 5 g/L urea, 1 g/L KH2PO4, 1 g/L K2HPO4, 0.25 g/L MgSO4·7H2O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment, lactose (20 g/L) was added to the medium.

The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added.

Complex medium, e.g., TY, was sterilized by autoclaving (121° C., 21′) and minimal medium by filtration (0.22 μm Sartorius). When necessary, the medium was made selective by adding an antibiotic (e.g., kanamycin, ampicillin).

Strain

Corynebacterium glutamicum ATCC 13032, available at the American Type Culture Collection.

Plasmids

Integrative plasmid vectors based on the Cre/loxP technique as described by Suzuki et al. (Appl. Microbiol. Biotechnol., 2005 Apr. 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example to produce LN3, C. glutamicum mutant strains are created to contain a gene coding for a lactose importer (such as the E. coli lacY with SEQ ID NO: 52) and further contain a constitutive transcriptional unit comprising a galactoside beta-1,3-N-acetylglucosaminyltransferase like e.g., lgtA from N. meningitidis (SEQ ID NO: 22). In an example for LNT production, the LN3 producing strain is further modified with a constitutive transcriptional unit comprising an N-acetylglucosamine beta-1,3-galactosyltransferase like e.g., WbgO from E. coli O55:H7 (SEQ ID NO: 23). In an example for LNnT production, the LN3 producing strain is further modified with a constitutive transcriptional unit comprising an N-acetylglucosamine beta-1,4-galactosyltransferase like e.g., lgtB from N. meningitidis (SEQ ID NO: 24). To further produce fucosylated oligosaccharides with LN3 as a core trisaccharide, C. glutamicum cells can be modified with an expression plasmid (or via a genomic knock-in) comprising a constitutive transcriptional unit for an alpha-1,2-fucosyltransferase like e.g., HpFutC from H. pylori (SEQ ID NO: 29) and/or an alpha-1,3-fucosyltransferase like e.g., HpFucT from H. pylori (SEQ ID NO: 30).

In a next step, the membrane proteins with SEQ ID NO: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 and 98 as enlisted in Tables 2 and 3 can be evaluated in above mutant strains. Membrane protein genes can be evaluated either present on a suitable expression plasmid as described herein or integrated in the host's genome in constitutive transcriptional units.

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer.

Analytical Analysis

It is referred to this section of Example 4.

### Example 30. Membrane Proteins Tested for LN3 Production in a C. glutamicum Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production of a host cell growing in media (see Example 29) supplemented with 20 g/L lactose. Each strain is modified for the production of LN3 as described in Example 29. In addition, each strain grows on sucrose by genomic knock-out of the nagB, glmS and gamA genes and genomic knock-ins of constitutive transcriptional units comprising genes encoding the lactose permease (LacY) from E. coli (SEQ ID NO: 52), the native fructose-6-P-aminotransferase (UniProt ID Q8NND3), the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6). Candidate membrane protein genes were presented to the LN3 production hosts by genomic knock-in of a constitutive transcriptional unit comprising the membrane protein gene. A growth experiment was performed according to the cultivation conditions provided in Example 29. The production of LN3 was evaluated in each LN3 production host expressing the galactoside beta-1,3-N-acetylglucosaminyltransferase (LgtA) from N. meningitidis with SEQ ID NO: 22.

### Example 31. Membrane Proteins Tested for Lacto-N-Tetraose (LNT) Production in a C. glutamicum Host

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-tetraose (LNT) production of a host cell growing in media (see Example 29) supplemented with 20 g/L lactose. Each strain is modified for the production of LNT as described in Example 29. In addition, each strain grows on sucrose by genomic knock-out of the nagB, glmS and gamA genes and genomic knock-ins of constitutive transcriptional units comprising genes encoding the lactose permease (LacY) from E. coli (SEQ ID NO: 52), the native fructose-6-P-aminotransferase (UniProt ID Q8NND3), the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6). Candidate genes were presented to the LNT production hosts by genomic knock-in of a constitutive transcriptional unit comprising the membrane protein gene. A growth experiment was performed according to the cultivation conditions provided in Example 29. The production of LN3 and LNT was evaluated in each LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

### Example 32. Membrane Proteins Identified that Enhance Lacto-N-Neotetraose (LNnT) Production in a C. glutamicum Host

An experiment was set up to evaluate membrane proteins with SEQ ID NOs SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-neotetraose (LNnT) production of a host cell growing in media (see Example 29) supplemented with 20 g/L lactose. Each strain is modified for the production of LNnT as described in Example 29. In addition, each strain grows on sucrose by genomic knock-out of the nagB, glmS and gamA genes and genomic knock-ins of constitutive transcriptional units comprising genes encoding the lactose permease (LacY) from E. coli (SEQ ID NO: 52), the native fructose-6-P-aminotransferase (UniProt ID Q8NND3), the sucrose transporter (CscB) from E. coli W (UniProt ID EOIXR1), the fructose kinase (Frk) from Z. mobilis (UniProt ID Q03417) and the sucrose phosphorylase (BaSP) from B. adolescentis (UniProt ID AOZZH6). Candidate genes were presented to the LNnT production hosts by genomic knock-in of a constitutive transcriptional unit comprising the membrane protein gene. A growth experiment was performed according to the cultivation conditions provided in Example 29. The production of LN3 and LNnT was evaluated in each LNnT production host expressing the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

### Example 33. Materials and Methods Animal Cells

Isolation of Mesenchymal Stem Cells from Adipose Tissue of Different Mammals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/ml epithelial growth factor and 5 pg/ml insulin. At confluence, cells were fed with growth medium supplemented with 2% fetal bovine serum, 1% penicillin-streptomycin (100 U/ml penicillin, 100 ug/ml streptomycin), and 5 pg/ml insulin for 48 h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/ml epithelial growth factor and 5 pg/ml insulin. At confluence, cells were fed with growth medium supplemented with 2% fetal bovine serum, 1% penicillin-streptomycin (100 U/ml penicillin, 100 ug/ml streptomycin), and 5 pg/ml insulin for 48 h. To induce differentiation, the cells were fed with complete growth medium containing 5 pg/ml insulin, 1 pg/ml hydrocortisone, 0.65 ng/ml triiodothyronine, 100 nM dexamethasone, and 1 pg/ml prolactin. After 24 h, serum is removed from the complete induction medium.

Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling is performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO21067641, which is incorporated herein by reference in its entirety for all purposes.

Cultivation

Completed growth media includes high glucose DMEM/F12, 10% FBS, 1% NEAA, 1% pen/strep, 1% ITS-X, 1% F-Glu, 10 ng/ml EGF, and 5 pg/ml hydrocortisone. Completed lactation media includes high glucose DMEM/F12, 1% NEAA, 1% pen/strep, 1% ITS-X, 1% F-Glu, 10 ng/ml EGF, 5 pg/ml hydrocortisone, and 1 pg/ml prolactin (5 ug/ml in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

Analytical Analysis

It is referred to this section of Example 4.

### Example 34. Membrane Proteins Tested for LN3 Production in a Non-Mammary Adult Stem Cell

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 production. Isolated mesenchymal cells and re-programmed into mammary-like cells as described in Example 33 are modified via CRISPR-CAS to over-express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase (UniProt ID Q16222) and the galactoside beta-1,3-N-acetylglucosaminyltransferase LgtA from N. meningitidis with SEQ ID NO: 22. All genes introduced are codon-optimized to the host cells. Further, candidate membrane protein genes were introduced to the LN3 production hosts (CRISPR-CAS). A growth experiment was performed according to the cultivation conditions provided in Example 33. The production of LN3 was evaluated in each LN3 production host expressing the galactoside beta-1,3-N-acetylglucosaminyltransferase (LgtA) from N. meningitidis with SEQ ID NO: 22.

### Example 35. Membrane Proteins Tested for Lacto-N-Tetraose (LNT) Production in a Non-Mammary Adult Stem Cell

An experiment was set up to evaluate membrane proteins with SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-tetraose (LNT) production. Isolated mesenchymal cells and re-programmed into mammary-like cells as described in Example 33 are modified via CRISPR-CAS to over-express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase (UniProt ID Q16222), the galactoside beta-1,3-N-acetylglucosaminyltransferase LgtA from N. meningitidis with SEQ ID NO: 22 and the N-acetylglucosamine beta-1,3-galactosyltransferase (wbgO) from N. meningitidis with SEQ ID NO: 23. All genes introduced are codon-optimized to the host cells. Further, candidate membrane protein genes were introduced to the LNT production hosts (CRISPR-CAS). A growth experiment was performed according to the cultivation conditions provided in Example 33. The production of LN3 and LNT was evaluated in each LNT production host expressing the galactoside beta-1,3-N-acetylglucosaminyltransferase (LgtA) from N. meningitidis with SEQ ID NO: 22 and the N-acetylglucosamine beta-1,3-galactosyltransferase (wbgO) from N. meningitidis with SEQ ID NO: 23.

### Example 36. Membrane Proteins Identified that Enhance Lacto-N-Neotetraose (LNnT) Production in a Non-Mammary Adult Stem Cell

An experiment was set up to evaluate membrane proteins with SEQ ID NOs SEQ ID NOs: 01, 02, 03, 04, 05, 06, 07, 08, 09, 10, 11, 12, 13, 14, 15, 16, 17, 18, 19, 20, 21, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97 or 98 for their ability to enhance LN3 and/or lacto-N-neotetraose (LNnT) production. Isolated mesenchymal cells and re-programmed into mammary-like cells as described in Example 33 are modified via CRISPR-CAS to over-express the GlcN6P synthase from Homo sapiens (UniProt ID Q06210), the glucosamine 6-phosphate N-acetyltransferase from Homo sapiens (UniProt ID Q96EK6), the phosphoacetylglucosamine mutase from Homo sapiens (UniProt ID 095394), the UDP-N-acetylhexosamine pyrophosphorylase (UniProt ID Q16222), the galactoside beta-1,3-N-acetylglucosaminyltransferase LgtA from N. meningitidis with SEQ ID NO: 22 and the N-acetylglucosamine beta-1,4-galactosyltransferase (LgtB) from N. meningitidis with SEQ ID NO: 24. All genes introduced are codon-optimized to the host cells. Further, candidate membrane protein genes were introduced to the LNnT production hosts (CRISPR-CAS). A growth experiment was performed according to the cultivation conditions provided in Example 33. The production of LN3 and LNnT was evaluated in each LNnT production host expressing the galactoside beta-1,3-N-acetylglucosaminyltransferase (LgtA) from N. meningitidis with SEQ ID NO: 22 and the N-acetylglucosamine beta-1,4-galactosyltransferase (LgtB) from N. meningitidis with SEQ ID NO: 24.

1.-45. (canceled)

46. A host cell genetically modified to produce an oligosaccharide comprising a lacto-N-triose (LN3; GlcNAc-beta1,3-Gal-beta1,4-Glc) as a core trisaccharide, wherein the host cell comprises and expresses at least one polynucleotide that encodes a galactoside beta-1,3-N-acetylglucosaminyltransferase that transfers an N-acetylglucosamine (GlcNAc) residue from a UDP-GlcNAc donor to a lactose acceptor thereby synthesizing LN3,
wherein the cell i) overexpresses an endogenous membrane protein and/or ii) expresses a heterologous membrane protein providing a) improved production and/or b) enabled and/or enhanced efflux, of an oligosaccharide comprising LN3 as a core trisaccharide,
optionally the cell further comprising and expressing at least one polynucleotide encoding for a glycosyltransferase that is capable of modifying the LN3, and
optionally wherein the improved production comprises:
better titer of the oligosaccharide (gram oligosaccharide per liter),
better production rate r (gram oligosaccharide per liter per hour),
better cell performance index (gram oligosaccharide per gram biomass),
better specific productivity (gram oligosaccharide per gram biomass per hour),
better yield on sucrose (gram oligosaccharide per gram sucrose),
better sucrose uptake/conversion rate (gram sucrose per gram per hour),
better lactose conversion/consumption rate (gram lactose per hour), and/or
enhanced growth speed of the host cell.

47. The cell of claim 46, wherein the improved production and the enhanced efflux is compared to a host cell with an identical genetic background but lacking the overexpression of an endogenous membrane protein and the expression of a heterologous membrane protein.

48. The cell of claim 46, wherein the membrane protein is selected from the group of porters, P-P-bond-hydrolysis-driven transporters, and β-Barrel Porins, wherein
a) when the membrane protein is selected from the group of porters, the membrane protein is selected from
the group of TCDB classes 2.A.1.1, 2.A.1.2, 2.A.1.3, 2.A.1.6, 2.A.2.2, 2.A.7.1 and 2.A.66, or
the group of eggnog families 5E8G, 5EGZ, 5JHE, 7QF7 7QRN, 7RBJ, 814C and 8N8 Å, or
the PFAM list PF00893, PF01943, PF05977, PF07690 and PF13347, or
the interpro list IPR000390, IPR001411, IPR001927, IPR002797, IPR004638, IPR005829, IPR010290, IPR011701, IPR020846, IPR023721, IPR023722, IPR032896, IPR036259 and IPR039672, or
MdfA from Cronobacter muytjensii with SEQ ID NO: 1, MdfA from Yokenella regensburgei (ATCC43003) with SEQ ID NO: 2, MdfA from Escherichia coli K-12 MG1655 with SEQ ID NO: 3, MdfA from Enterobacter sp. with SEQ ID NO: 4, MFS from Citrobacter koseri with SEQ ID NO: 5, MdfA from Citrobacter youngae with SEQ ID NO: 6, YbdA from Escherichia coli K-12 MG1655 with SEQ ID NO: 7, YjhB from Escherichia coli K-12 MG1655 with SEQ ID NO: 8, WzxE from Escherichia coli K-12 MG1655 with SEQ ID NO: 9, EmrE from Escherichia coli K-12 MG1655 with SEQ ID NO: 10, Blon_2331 from Bifidobacterium longum subsp. Infantis (strain ATCC 15697) with SEQ ID NO: 11, Blon_0247 from Bifidobacterium longum subsp. Infantis (strain ATCC 15697) with SEQ ID NO: 12, Blon_0345 from Bifidobacterium longum subsp. Infantis (strain ATCC 15697) with SEQ ID NO: 13, IceT from Klebsiella pneumoniae with SEQ ID NO: 14, MdfA from Cronobacter sakazakii strain MOD1_LR753 with SEQ ID NO: 53, MdfA from Franconibacter pulveris LMG 24059 with SEQ ID NO: 54, MdfA from Enterobacter hormaechei strain 17 with SEQ ID NO: 55, MdfA from Citrobacter koseri strain NCTC10771 with SEQ ID NO: 56, MdfA from Salmonella enterica subsp. arizonae serovar 41:z4,z23:-strain TAMU30EF with SEQ ID NO: 57, MdfA from Shigella flexneri strain 585219 with SEQ ID NO: 58, MdfA from Yokenella regensburgei strain UMB0819 with SEQ ID NO: 59, MdfA from Escherichia coli strain AMC_967 with SEQ ID NO: 60, MdfA from Klebsiella pneumoniae VAKPC309 with SEQ ID NO: 61, MdfA from Klebsiella oxytoca strain 4928STDY7071490 with SEQ ID NO: 62, MdfA from Klebsiella michiganensis strain A2 with SEQ ID NO: 63, MdfA from Pluralibacter gergoviae strain FDAARGOS_186 with SEQ ID NO: 64, MdfA from Kluyvera ascorbata ATCC 33433 with SEQ ID NO: 65, MdfA from Enterobacter kobei with SEQ ID NO: 66, MdfA from Lelliottia sp. WB101 with SEQ ID NO: 67, MdfA from Citrobacter freundii with SEQ ID NO: 68, MdfA from Salmonella enterica subsp. Salamae with SEQ ID NO: 69 or MdfA from Shigella flexneri with SEQ ID NO: 70, or functional homolog or functional fragment of any one of the above porter membrane proteins or a protein sequence having at least 80%, sequence identity to the full-length sequence of any one of the membrane proteins with SEQ ID NO: 1, 2, 3, 4, 5, 6, 7, 8, 9, 10, 11, 12, 13, 14, 53, 54, 55, 56, 57, 58, 59, 60, 61, 62, 63, 64, 65, 66, 67, 68, 69, or 70, respectively;
b) when the membrane protein is selected from the group of P-P-bond-hydrolysis-driven transporters, the membrane protein is selected from
the group of TCDB classes 3.A.1.1 and 3.A.1.2, or
the group of eggnog families 5CJ1, 5DFW, 5EZD, 5I1K, 7HR3 and 8IJ9, or
the PFAM list PF00005, PF00528, PF13407 and PF17912, or
the interpro list IPR000515, IPR003439, IPR003593, IPR005978, IPR008995, IPR013456, IPR015851, IPR017871, IPR025997, IPR027417, IPR028082, IPR035906, and IPR040582, or
Blon_2475 from Bifidobacterium longum subsp. Infantis (strain ATCC 15697) with SEQ ID NO: 15, nodi from Bradyrhizobium japonicum USDA 110 with SEQ ID NO: 16, xylF from Escherichia coli K-12 MG1655 with SEQ ID NO: 17, TIC77290 from Bifidobacterium longum subsp. Infantis Bi-26 with SEQ ID NO: 18, TIC77291 from Bifidobacterium longum subsp. Infantis Bi-26 with SEQ ID NO: 19, TIC76854 TIC77291 from Bifidobacterium longum subsp. Infantis Bi-26 with SEQ ID NO: 20 or functional homolog or functional fragment of any one of the above P-P-bond-hydrolysis-driven transporter membrane proteins or a protein sequence having at least 80%, sequence identity to the full-length sequence of any one of the membrane proteins with SEQ ID NO: 15, 16, 17, 18, 19, or 20, respectively; or
c) when the membrane protein is selected from the group of β-Barrel Porins, the membrane protein is selected from
TCDB class 1.B.18, or
eggnog family 5DAY, or
PFAM list PF02563, PF10531 and PF18412, or
the interpro list IPR003715, IPR019554 and IPR040716, or
Wza from Escherichia coli K12 MG1655 with SEQ ID NO: 21 or functional homolog or functional fragment thereof, or a protein sequence having at least 80%, sequence identity to the full-length sequence of the membrane protein with SEQ ID NO: 21;
wherein the TCDB classes are as defined by TCDB.org as released on 17 Jun. 2019, the eggnog families are as defined by eggnogdb 4.5.1 as released on September 2016, the PFAM lists are as defined by Pfam 32.0 as released on September 2018, the interpro lists are as defined by InterPro 75.0 as released on 4 Jul. 2019.

49. The cell of claim 46, wherein the membrane protein is selected from the group of membrane proteins consisting of
a) the porter membrane proteins represented by SEQ ID NO: 1, 2, 4, 5, 6, 9, 10, 11, 12, 13, 14, 53, 54, 55, 56, 57, 58, 59, 60, 61, 62, 63, 64, 65, 66, 67, 68, 69, or 70, or functional homolog or functional fragment of any one of the porter membrane proteins, or a protein sequence having at least 80% sequence identity to the full-length sequence of the membrane proteins with SEQ ID NOs: 1, 2, 4, 5, 6, 9, 10, 11, 12, 13, 14, 53, 54, 55, 56, 57, 58, 59, 60, 61, 62, 63, 64, 65, 66, 67, 68, 69 or 70, respectively;
b) the P-P-bond-hydrolysis-driven transporters represented by SEQ ID NO: 15, 16, 17, 18, 19, or 20, or functional homolog or functional fragment of any one of the P-P-bond-hydrolysis-driven transporter membrane proteins, or a protein sequence having at least 80%, sequence identity to the full-length sequence of the membrane proteins with SEQ ID NOs: 15, 16, 17, 18, 19 or 20, respectively; and
c) the β-barrel porin membrane protein represented by SEQ ID NO: 21, or functional homolog or functional fragment of the β-barrel porin membrane protein, or a protein sequence having at least 80%, sequence identity to the full-length sequence of the membrane protein with SEQ ID NO: 21.

50. The cell of claim 46, wherein the membrane protein is selected from the group of membrane proteins consisting of
a) the porter membrane proteins represented by SEQ ID NO: 1, 2, 4, 5, 6, 9, 10, 11, 12, 13, 14, 53, 54, 55, 56, 57, 58, 59, 60, 61, 62, 63, 64, 65, 66, 67, 68, 69, or 70, or functional homolog or functional fragment of any one of the porter membrane proteins, or a protein sequence having:
at least 80% sequence identity to the full-length sequence of the membrane proteins with SEQ ID NOs: 9, 10, 11, 12 or 13, respectively,
at least 90% sequence identity to the full-length sequence of the membrane proteins with SEQ ID NOs: 1, 2, 4, 14, 53, 54, 55, 59, 61, 62, 63, 64, 65, 66, 67 or 69, respectively,
at least 95.00% sequence identity to the full-length sequence of the membrane proteins with SEQ ID NOs: 5, 6, 56, 57 or 68, respectively, or
at least 99.00% sequence identity to the full-length sequence of the membrane proteins with SEQ ID NOs: 58, 60 or 70, respectively; and
b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, sequence identity to the full-length sequence of the membrane proteins with SEQ ID NOs: 15, 16, 17, 18, 19 or 20, respectively; and
c) the β-barrel porin membrane protein represented by SEQ ID NO: 21, or functional homolog or functional fragment of the β-barrel porin membrane protein, or a protein sequence having at least 80%, sequence identity to the full-length sequence of the membrane protein with SEQ ID NO: 21.

51. The cell of claim 46, wherein the membrane protein is selected from the group of membrane proteins consisting of
a) the porter membrane proteins represented by SEQ ID NO: 1, 2, 4, 9, 10, 11, 12, 13, 14, 53, 54, 55, 59, 61, 62, 63, 64, 65, 66, 67, or 69, or functional homolog or functional fragment of any one of the porter membrane proteins, or a protein sequence having at least 90%, sequence identity to the full-length sequence of the membrane proteins with SEQ ID NOs: 1, 2, 4, 9, 10, 11, 12, 13, 14, 53, 54, 55, 59, 61, 62, 63, 64, 65, 66, 67, or 69, respectively; and
b) the P-P-bond-hydrolysis-driven transporters represented by SEQ ID NO: 15, 16, 17, 18, 19 or 20, or functional homolog or functional fragment of any one of the P-P-bond-hydrolysis-driven transporter membrane proteins, or a protein sequence having at least 80%, sequence identity to the full-length sequence of the membrane proteins with SEQ ID NOs: 15, 16, 17, 18, 19 or 20, respectively; and
c) the β-barrel porin membrane protein represented by SEQ ID NO: 21, or functional homolog or functional fragment of the β-barrel porin membrane protein, or a protein sequence having at least 80%, sequence identity to the full-length sequence of the membrane protein with SEQ ID NO: 21.

52. The cell of claim 46, wherein the oligosaccharide comprising a lacto-N-triose (LN3; GlcNAc-beta1,3-Gal-beta1,4-Glc) as a core trisaccharide is a mammalian milk oligosaccharide or a Lewis-type antigen oligosaccharide.

53. The cell of claim 46, wherein the oligosaccharide comprising a lacto-N-triose (LN3; GlcNAc-beta1,3-Gal-beta1,4-Glc) as a core trisaccharide is a neutral oligosaccharide.

54. The cell of claim 46, wherein the oligosaccharide comprising a lacto-N-triose (LN3; GlcNAc-beta1,3-Gal-beta1,4-Glc) as a core trisaccharide is selected from the group consisting of lacto-N-triose, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, lacto-N-hexaose (LNH), lacto-N-neohexaose (LNnH), para-lacto-N-hexaose (pLNnH), para-lacto-N-neohexaose (pLNH), difucosyl-lacto-N-hexaose, difucosyl-lacto-N-neohexaose, lacto-N-pentaose (LNP), lacto-N-neopentaose, para lacto-N-pentaose, para lacto-N-neopentaose, lacto-N-novopentaose I, lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, Sialyl-lacto-N-tetraose a, Sialyl-lacto-N-tetraose b, Sialyl-lacto-N-tetraose c, Sialyl-lacto-N-tetraose d.

55. The cell of claim 46, wherein the glycosyltransferase is an N-acetylglucosamine beta-1,3-galactosyltransferase or an N-acetylglucosamine beta-1,4-galactosyltransferase that transfers a galactose (Gal) from a UDP-Gal donor to the terminal GlcNAc residue of LN3 in a beta-1,3 or beta-1,4 linkage, thereby producing lacto-N-tetraose (LNT; Gal-beta1,3-GlcNAc-beta1,3-Gal-beta1,4-Glc) or lacto-N-neotetraose (LNnT; Gal-beta1,4-GlcNAc-beta1,3-Gal-beta1,4-Glc), respectively.

56. The cell of claim 46, wherein the cell produces:
90 g/L or more of LNT in the whole broth and/or the supernatant and/or wherein the LNT in the whole broth and/or the supernatant has a purity of at least 80% measured on the total amount of LNT and LN3 produced by the cell in the whole broth and/or the supernatant, respectively; or
70 g/L or more, of LNnT in the whole broth and/or the supernatant and/or wherein the LNnT in the whole broth and/or the supernatant has a purity of at least 80% measured on the total amount of LNnT and LN3 produced by the cell in the whole broth and/or the supernatant, respectively.

57. The cell of claim 46, wherein the cell is a microorganism, a plant cell, an animal cell, an insect cell, or a protozoan cell.

58. The cell of claim 57, wherein the cell is a cell of a bacterium.

59. The cell of claim 46, wherein the cell is capable of synthesizing a mixture of oligosaccharides comprising at least one oligosaccharide comprising LN3 as a core trisaccharide.

60. A method for producing (i) an oligosaccharide comprising a lacto-N-triose (LN3; GlcNAc-beta1,3-Gal-beta1,4-Glc) as a core trisaccharide or (ii) an oligosaccharide mixture comprising at least one oligosaccharide comprising a lacto-N-triose (LN3; GlcNAc-beta1,3-Gal-beta1,4-Glc) as a core trisaccharide by a genetically modified cell, comprising:
a) culturing the cell of claim 46 in a medium under conditions permissive for producing the oligosaccharide comprising LN3 as a core trisaccharide or the oligosaccharide mixture comprising at least one oligosaccharide comprising LN3 as a core trisaccharide, and
b) optionally separating the oligosaccharide comprising LN3 as a core trisaccharide or the oligosaccharide mixture comprising at least one oligosaccharide comprising LN3 as a core trisaccharide, respectively from the cultivation.

61. The method according to claim 60, the method further comprising at least one of the following steps:
i) adding to the culture medium a lactose feed comprising at least 50 grams of lactose per liter of initial reactor volume wherein the reactor volume ranges from 250 mL to 10.000 m³ (cubic meter);
ii) adding a lactose feed in a continuous manner to the culture medium over the course of 1 day, 2 days, 3 days, 4 days, 5 days by means of a feeding solution; and
iii) adding a lactose feed in a continuous manner to the culture medium over the course of 1 day, 2 days, 3 days, 4 days, 5 days by means of a feeding solution and wherein the concentration of the lactose feeding solution is 50 g/L;
wherein the method results in an oligosaccharide comprising a lacto-N-triose (LN3; GlcNAc-beta1,3-Gal-beta1,4-Glc) as a core trisaccharide with a concentration of at least 50 g/L in the final volume of the culture medium.

62. The method according to claim 60, wherein the host cells are cultivated for at least about 60 hours or in a continuous manner.

63. The method according to claim 60, wherein separation comprises at least one of the following steps: clarification, ultrafiltration, nanofiltration, reverse osmosis, microfiltration, activated charcoal or carbon treatment, tangential flow high-performance filtration, tangential flow ultrafiltration, affinity chromatography, ion exchange chromatography, hydrophobic interaction chromatography, and/or gel filtration, ligand exchange chromatography.

64. The method according to claim 60, further comprising purification of the oligosaccharide comprising LN3 as a core trisaccharide or the oligosaccharide mixture comprising at least one oligosaccharide comprising LN3 as a core trisaccharide, respectively from the cell.

65. The method according to claim 60, wherein the purification comprises at least one of the following steps: using activated charcoal or carbon, using charcoal, nanofiltration, ultrafiltration or ion exchange, using alcohols, using aqueous alcohol mixtures, crystallization, evaporation, precipitation, drying, spray drying, or lyophilization.


SEQUENCE LISTING (98 sequences)

SEQ ID NO: 1 - protein, 410 aa, Cronobacter muytjensii
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALILGNLVLAKLTARRSVRSLIIMGGWPIMAGLALAAFATLLSSHAYLWMTAGLSIYAFGIGIANAGLVRLTLFASDISKGTVSAAMGMLQMTIFTVGIEVSKHAWIGGGNGLFNLFNLANGLLWLGLMVIFLKDKTVGASREG

SEQ ID NO: 2 - protein, 411 aa, Yokenella regensburgei (ATCC43003)
MQNHTLSGKRLGRQALLFPLCLVLYEFSTYIGNDMIQPGMLAVVEQYQAGIEWVPTSMTAYLAGGIFLQWLLGPLSDRIGRRPVMLTGVVWFIVTCLATLLAQNIEQFTFLRFLQGVSLCFIGAVGYAAIQESFEEAVCIKITALMANVALIAPLLGPLVGAAWIHFAPWETMFVLFAALAAISFFGLQRAMPETATRIGEKLSLKELGRDYSAVLKNLRFVAGALATGFVSLPLLAWIAQSPVIIMSGEHASSYEYGMLQVPIFGALIIGNLVLARLTSRRSVRSLIIMGGWPIVAGLILAAAATVVSSHAYLWMTAGLSVYAFGIGLANAGLVRLTLFASDMSKGTVAAAMGMLQMMIFTVGIEVSKHAYLLGGNGLFSLFNLAGGLVWLVLIVYFLKDKTVGASTKPD

SEQ ID NO: 3 - protein, 410 aa, Escherichia coli strain K12 (MG1655)
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 4 - protein, 411 aa, Enterobacter sp.
MLNRSSSGTRLGRQALLFPLCLVLYEFSTYIGNDMIQPGMLAVVAQYNAGIEWVPTSMTAYLAGGMFLQWLLGPLSDRIGRRPVMLTGVAWFIVTCLATLLAQTIEQFMVLRFLQGVSLCFIGAVGYAAIQESFEEAVCIKITALMANVALIAPLLGPLVGAAWVHVAPWEGMFVLFAVLAAISFYGLHRAMPETATRIGEKLSLQELGRDYKEVLKNGRFVAGALAIGFVCLPLLAWIAQSPVIIISGENLSSYEYGLLQVPIFGALIVGNIVLARLTSRRTVRSLIIMGGWPIVIGLVVAAVATVVSSHAYLWMTAGLSIYAFGIGLANAGLVRLTLFASEVSKGTVSAAMGMLQMLIFTVGIEVSKHAFSSGGNGLFSLFNLVNGLLWLALMFVFLKDKRVGSSLQPG

SEQ ID NO: 5 - protein, 410 aa, Citrobacter koseri (Citrobacter diversus)
MQNLSQTGVRLGRQALLFPLCLVLYEFSTYIGNDMIQPGMLAVVEQYQAGIDWVPTSMTAYLAGGMFLQWLLGPLSDRIGRRPVMLAGVIWFIVTCLATLLAQNIEQFTLLRFLQGVSLCFIGAVGYAAIQESFEEAVCIKITALMANVALIAPLLGPLVGAAWVHVLPWEGMFVLFAVLAAIAFVGLQRAMPETATRLGEKLSLKELGRDYTLVLKNVRFVAGALALGFVSLPLLAWIAQSPIIIISGEQLSSYEYGLLQVPVFGALIAGNLVLARLTSRRTVRALIIMGGWPIVAGLLIAAAATVVSSHAYLWMTAGLSVYAFGIGVANAGLVRLTLFASDMSKGTVSAAMGMLQMLIFTVGIEVSKHAYLSGGNGLFSLFNLANGILWLLLMVIFLKDKRVGDSREG

SEQ ID NO: 6 - protein, 410 aa, Citrobacter youngae ATCC 29220
MQNRLSSGARLGRQALLFPLCLVLYEFSTYIGNDMIQPGMLAVVEQYNAGLDWVPTSMTAYLAGGMFLQWLLGPLSDRIGRRPVMLTGVLWFIVTCLATLLAQNIEQFTFLRFLQGISLCFIGAVGYAAIQESFEEAVCIKITALMANVALIAPLLGPLVGAAWVHILPWEGMFILFAALASISFFGLQRAMPETATRLGEKLSIKELGKDYKLVLKNGRFVAGALALGFVSLPLLAWIAQSPIIIISGEHLSSYEYGLLQVPIFGALIAGNLALARLTSRKTVRSLIIMGGWPIAVGLVIAAAATVVSSHAYLWMTAGLSVYAFGIGLANAGLVRLTLFASEMSKGTVSAAMGMLQMLIFTVGIELSKHAYLLGGNGLFSLFNLASGVLWLILMVIFLKDKRVGNSRKI

SEQ ID NO: 7 - protein, 416 aa, Escherichia coli K12 MG1655
MNKQSWLLNLSLLKTHPAFRAVFLARFISIVSLGLLGVAVPVQIQMMTHSTWQVGLSVTLTGGAMFVGLMVGGVLADRYERKKVILLARGTCGIGFIGLCLNALLPEPSLLAIYLLGLWDGFFASLGVTALLAATPALVGRENLMQAGAITMLTVRLGSVISPMIGGLLLATGGVAWNYGLAAAGTFITLLPLLSLPALPPPPQPREHPLKSLLAGFRFLLASPLVGGIALLGGLLTMASAVRVLYPALADNWQMSAAQIGFLYAAIPLGAAIGALTSGKLAHSARPGLLMLLSTLGSFLAIGLFGLMPMWILGVVCLALFGWLSAVSSLLQYTMLQTQTPEAMLGRINGLWTAQNVTGDAIGAALLGGLGAMMTPVASASASGFGLLIIGVLLLLVLVELRHFRQTPPQVTASDS

SEQ ID NO: 8 - protein, 405 aa, Escherichia coli K12 MG1655
MATAWYKQVNPPQRKALFSAWLGYVFDGFDFMMIFYILHIIKADLGITDIQATLIGTVAFIARPIGGGFFGAMADKYGRKPMMMWAIFIYSVGTGLSGIATNLYMLAVCRFIVGLGMSGEYACASTYAVESWPKNLQSKASAFLVSGFSVGNIIAAQIIPQFAEVYGWRNSFFIGLLPVLLVLWIRKSAPESQEWIEDKYKDKSTFLSVFRKPHLSISMIVFLVCFCLFGANWPINGLLPSYLADNGVNTVVISTLMTIAGLGTLTGTIFFGFVGDKIGVKKAFVVGLITSFIFLCPLFFISVKNSSLIGLCLFGLMFTNLGIAGLVPKFIYDYFPTKLRGLGTGLIYNLGATGGMAAPVLATYISGYYGLGVSLFIVTVAFSALLILLVGFDIPGKIYKLSVAK

SEQ ID NO: 9 - protein, 416 aa, Escherichia coli K12 MG1655
MSLAKASLWTAASTLVKIGAGLLVGKLLAVSFGPAGLGLAANFRQLITVLGVLAGAGIFNGVTKYVAQYHDNPQQLRRVVGTSSAMVLGFSTLMALVFVLAAAPISQGLFGNTDYQGLVRLVALVQMGIAWGNLLLALMKGFRDAAGNALSLIVGSLIGVLAYYVSYRLGGYEGALLGLALIPALVVIPAAIMLIKRGVIPLSYLKPSWDNGLAGQLSKFTLMALITSVTLPVAYIMMRKLLAAQYSWDEVGIWQGVSSISDAYLQFITASFSVYLLPTLSRLTEKRDITREVVKSLKFVLPAVAAASFTVWLLRDFAIWLLLSNKFTAMRDLFAWQLVGDVLKVGAYVFGYLVIAKASLRFYILAEVSQFTLLMVFAHWLIPAHGALGAAQAYMATYIVYFSLCCGVFLLWRRRA

SEQ ID NO: 10 - protein, 110 aa, Escherichia coli K12 MG1655
MNPYIYLGGAILAEVIGTTLMKFSEGFTRLWPSVGTIICYCASFWLLAQTLAYIPTGIAYAIWSGVGIVLISLLSWGFFGQRLDLPAIIGMMLICAGVLIINLLSRSTPH

SEQ ID NO: 11 - protein, 507 aa, Bifidobacterium longum subsp. Infantis (strain ATCC 15697)
MSNENTAVGDVRKKGGLGQRIAYACGNLGQAAFYNAMSTYFVTYVTSCLFVSYSKALAAQMIAVITGLIVVIRIAEIFIDPLLGNLVDNTTTKWGRFRPWQFIGGLVSSVLIMLIFSGMFGLVNVNTTLFIVLFVITFIVLDVFYSLRDISYWGMIPALSSDSHERSTYTALGTFTGSIGYNGITVIVIPIVSYFTWTFTGAKGQGQAGWTSFGFIVALLGLITAWAVAFGTKESTNALRAKAQKNGNPFEAFKALFQNDQLLWVALSYLLYAIANVITTGVMYYLFVFVLDEPAAFSVTGIIPLIAGFIMAPLYPILNRWIPRRYLFAGGMVSMIIGYTMLALFSSNLPVVIVALIFFYVPAQFIQMTAILSLTDSIEYGQLKNGKRNEAVTLSVRPMLDKIGGAMSNGVVGAVALAAGMTGHATAADMTASNITTFKTFAFYIPLVLIILSLVVFWFKVKIDEKMHAQIVDELEAKLASGEIVDDEAQTVEAVEAINEETPAAKN

SEQ ID NO: 12 - protein, 405 aa, Bifidobacterium longum subsp. Infantis (strain ATCC 15697)
MALDVGKALKSKTLVVGVLSMSLLMSASNAVSGTIPAMKEAFSDYSAANVELLTTVPTIGSMVGTALTGLFANAIGRKKIAMAGFLISAVTGVIPAFFPYYWPILISRMFFGFGSALFVTLSVSYITDLYDGDMQRKLLGWRQAVGNLGDVVLLFVASLLITINWQSTYLIFFLLFVPMVLVGMFIPKEFDNFSIRSALVDDEGHVVDKSASQKQTTNWQVLWLAFIFLVVSMLYNVMSIKLASYVVDEGIGSASLATLIFSFLVVATILSGVLFDKVAKVTKRLTVTISEVVIGICFIATALTKNVPLMFALVLIAGFAWGIINPALTARFVDYSPAHSMNLTTSIVIIGINIGCLISPYFFALTASIFGNSSAGFAIIVGGALYLVMVVIELITLKVDKKLTV

SEQ ID NO: 13 - protein, 407 aa, Bifidobacterium longum subsp. Infantis (strain ATCC 15697)
MSKIINYSEVLESKRLMVGVLSVSFLLSAGNAISGTIPAMEEAFSNISKANIETLTTIPTAGIMLGTVLSGVFSNYLGKKKSVLAGLIIALVGGVIPAFLPQYWPIFISRFLFGVGMGIFNPLSVSYITDLYVGDRQRSLLGYRNAVSNLGDTIMLFVAGILITFGWNITYLVFFALLIPIVLIILFVPKEFDNFDIRNSALDENGQISDSVSDVKPSTNLKVIEVGVVFMVITMLYNAIPLKFASYIVTEHIGTASTATWIFSFLVLAGIFSGVLFEKISKVFKRLTVFVFEIVIGVAYITIAFTYNIPLLTALVLISGFGWGIINPALTARLVDVSPINSMNLSTSIIVIFISVGSLISPYFFAMFAGLFGNDSAAFAIVVGGALYVVMAVLDFIKIKKNKELSI

SEQ ID NO: 14 - protein, 471 aa, Klebsiella pneumoniae
MTELPDSTRWQLWIVAFGFFMQSLDTTIVNTALPSMALSLGESPLHMHMVVVSYVLTVAVMLPASGWLADKVGVRNIFFTAIVLFTLGSLFCAWSSTLNELVLARVLQGVGGAMMVPVGRLTVMKIVPREQYMAAMTFVTLPGQVGPLLGPALGGILVEYASWHWIFLINIPVGIVGAIATLMLMPNYTMQTRRFDLSGFLLLAVGMAVLTLALDGSKGTGLSPLSLGALVLCGILAIALYLKHAKNNPRALFSLALFRTHTFSLGLSGSFAGRVGSGMLPFMTPVFLQIGLGFSPFHAGLMMIPMVLGSMGMKRIVVQVVNRFGYRRVLVSTTLGLSLVSLLFMSVAMLGWYYALPFVLFLQGMVNSTRFSSMNTLTLKDLPDELASSGNSLLSMIMQLSMSIGVTIAGLLLGMFGQQHIAADSGASHTVFMYTWLCMALIIALPALIFARVPNDTHKNAVISRRKRSTQ

SEQ ID NO: 15 - protein, 375 aa, Bifidobacterium longum subsp. Infantis (strain ATCC 15697)
MAEVVFDHVTRIYPGNDKPSVDDLNLDIKDGEFLVLVGPSGCGKSTTLRMLAGLEEVNKGRILIGGKDVTTMQPKDRDIAMVFQNYALYPHMTVADNMGFALKIAGTPKDEIRKRVEKAAEILDLTEYLDRKPKALSGGQRQRVAMGRAIVREPKVFLMDEPLSNLDAKLRVQTRTQIAALQRQLGVTTLYVTHDQTEALTMGDRIAVIKLGILQQVGAPTELYDRPANVFVAGFIGSPSMNLNTHPVVNGKAKIGEDTVDLPAEAVNKLTAEDNGQIVVGFRPEDAGLAPVDDPNAFSLKVVNVEDLGSDGYIYGTIVTDGSAAEASQVMSDQNKLTTIRVNPRALPKVGATVKIKIDPAKMHLFAPSTELRLN

SEQ ID NO: 16 - protein, 306 aa, Bradyrhizobium japonicum USDA 110
MNMSNMAIDLVGVRKSFGDKVIVNDLSFSVARGECFGLLGPNGAGKSTIARMLLGMISPDRGKITVLDEPVPSRARAARVRVGVVPQFDNLEPEFTVRENLLVFGRYFGMSARTIEAVVPSLLEFARLESKADVRVSLLSGGMKRRLTLARALINDPHLLVMDEPTTGLDPHARHLIWERLRALLARGKTILLTTHFMEEAERLCDRLCVLESGCKIAEGKPDALIDEHIGCNVIEIYGGDLDQLRELIRPYARHIEVSGETLFCYARCPDEISVHLRGRTDLRVLQRPPNLEDVFLRLTGREMEK

SEQ ID NO: 17 - protein, 330 aa, Escherichia coli K12 MG1655
MKIKNILLTLCTSLLLTNVAAHAKEVKIGMAIDDLRLERWQKDRDIFVKKAESLGAKVFVQSANGNEETQMSQIENMINRGVDVLVIIPYNGQVLSNVVKEAKQEGIKVLAYDRMINDADIDFYISFDNEKVGELQAKALVDIVPQGNYFLMGGSPVDNNAKLFRAGQMKVLKPYVDSGKIKVVGDQWVDGWLPENALKIMENALTANNNKIDAVVASNDATAGGAIQALSAQGLSGKVAISGQDADLAGIKRIAAGTQTMTVYKPITLLANTAAEIAVELGNGQEPKADTTLNNGLKDVPSRLLTPIDVNKNNIKDTVIKDGFHKESEL

SEQ ID NO: 18 - protein, 309 aa, Bifidobacterium longum infantis Bi-26
MSHATATKTAAKKPAKKKVSAFSTRKVDPAYYWMVVPAAIIFAFFLYLPFLDGVKYSFTNSQGYGDYKFIGLKNYIALFQDNRVGHAYLFTFLIAILITVLINVIALFLSVLLNSKIAFKNGFRAVFFIPYTLSVLVIGYVFKYIFMNPLPELGKALGIKWLSTSLLTNEQLSWIPIVFLAVWQGIAYSVLIYLAGLQTIDDEIYEAAAIDGVNAWQKFWKITFPLIGPFFTINLVLSMKNALGTFDQVVALTEGGPNSSTETVTYLIWKGGLTGGEYAYQTANAVLFFIVLAIIAFVQLRISRSQEQI

SEQ ID NO: 19 - protein, 288 aa, Bifidobacterium longum infantis Bi-26
MTTATVTPSKSGKPAKFRRDHKINWWLTAAVAVLSLTILIPLYFTIVTALKTPAEAGTFALPTSWQWHNFADASAKVNYPKAALNSAIITVAAVVLTLLTNTFVAYAVARNMDKRFFRFLYYFFIAAMFVPFPVVMLPIAKQMGSLHLDNQVGLIILYTVLGLGTNLFIATGFIRSIPVSLEEAARIDGASTWRIFWTIIFPLMSPINATIAILTALWAWNDFLLPLIILTDQSNQTIPLAQYVFSSQFATNYPMAFSSYLMAMAPILIVYIFAQKWVVGGVMRGAVK

SEQ ID NO: 20 - protein, 303 aa, Bifidobacterium longum infantis Bi-26
MTNATAQPDTSVMRKPKRQYIGILYCLPYVVVFLFGMIVPMFYALYLSFFKQSLLGGTTFAGFDNFIRAFKDEALWGGFKNVLIYAAIQIPMNLILSLVAALVLDSQRIRHIAVPRILLFLPYAVPGVIAALMWGYIYGDKYGLFGQIAGMFGVAAPNMLSKQLMLFAIANICTWCFLGYNMLIYYSALIGIPNDLYESARIDGASELRIAWSVKIPQIKSTIVMTVLFSVIGTLQLFNEPNILRTSAPDVINSSYTPNIYTYNLAFNGQNVNYAAAVSLVIGIIVMALVAVVKIIGNKWENK

SEQ ID NO: 21 - protein, 379 aa, Escherichia coli K12 MG1655
MMKSKMKLMPLLVSVTLISGCTVLPGSNMSTMGKDVIKQQDADFDLDKMVNVYPLTPRLIDQLRPRPNVARPNMTLESEIANYQYRVGPGDVLNVTVWDHPELTTPAGQYRSSSDTGNWVQPDGTMFYPYIGKVHVVGKTLAEIRSDITGRLATYIADPQVDVNIAAFRSQKAYISGQVNKSGQQAITNVPLTILDAINAAGGLTDTADWRNVVLTHNGREERISLQALMQNGDLNQNRLLYPGDILYVPRNDDLKVFVMGEVKKQSTLKMDFSGMTLTEALGNAEGIDMTTSNASGIFVIRPLKGEGGRNGKIANIYQLDMSDATSLVMATEFRLQPYDVVYVTTAPVSRWNRLINQLLPTISGVRYMTDTASDIHNW

SEQ ID NO: 22 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 23 - protein, 265 aa, Escherichia coli O55:H7
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 24 - protein, 275 aa, Neisseria meningitidis MC58
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 25 - protein, 609 aa, Escherichia coli K12 MG1655
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 26 - protein, 338 aa, Escherichia coli K12 MG1655
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 27 - protein, 445 aa, Escherichia coli K12 MG1655
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 28 - protein, 456 aa, Escherichia coli K12 MG1655
MLNNAMSVVILAAGKGTRMYSDLPKVLHTLAGKAMVQHVIDAANELGAAHVHLVYGHGGDLLKQALKDDNLNWVLQAEQLGTGHAMQQAAPFFADDEDILMLYGDVPLISVETLQRLRDAKPQGGIGLLTVKLDDPTGYGRITRENGKVTGIVEHKDATDEQRQIQEINTGILIANGADMKRWLAKLTNNNAQGEYYITDIIALAYQEGREIVAVHPQRLSEVEGVNNRLQLSRLERVYQSEQAEKLLLAGVMLRDPARFDLRGTLTHGRDVEIDTNVIIEGNVTLGHRVKIGTGCVIKNSVIGDDCEISPYTVVEDANLAAACTIGPFARLRPGAELLEGAHVGNFVEMKKARLGKGSKAGHLTYLGDAEIGDNVNIGAGTITCNYDGANKFKTIIGDDVFVGSDTQLVAPVTVGKGATIAAGTTVTRNVGENALAISRVPQTQKEGWRRPVKKK

SEQ ID NO: 29 - protein, 299 aa, Helicobacter pylori UA1234
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 30 - protein, 478 aa, Helicobacter pylori UA1234
MFQPLLDAYVESASIEKMASKSPPPLKIAVANWWGDEEIKEFKNSVLYFILSQRYTITLHQNPNEFSDLVFGNPLGSARKILSYQNAKRVFYTGENESPNFNLFDYAIGFDELDFNDRYLRMPLYYDRLHHKAESVNDTTAPYKLKDNSLYALKKPSHCFKEKHPNLCAVVNDESDPLKRGFASFVASNPNAPIRNAFYDALNSIEPVTGGGSVRNTLGYNVKNKNEFLSQYKFNLCFENTQGYGYVTEKIIDAYFSHTIPIYWGSPSVAKDFNPKSFVNVHDFKNFDEAIDYIKYLHTHKNAYLDMLYENPLNTLDGKAYFYQNLSFKKILAFFKTILENDTIYHDNPFIFCRDLNEPLVTIDDLRVNYDDLRVNYDDLRINYDDLRVNYDDLRINYDDLRVNYDDLRVNYDDLRINYDDLRVNYDDLRVNYERLLSKATPLLELSQNTTSKIYRKAYQKSLPLLRAIRRWVKKLGL

SEQ ID NO: 31 - protein, 264 aa, Escherichia coli K12 MG1655
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 32 - protein, 391 aa, Escherichia coli K12 MG1655
MQKLINSVQNYAWGSKTALTELYGMENPSSQPMAELWMGAHPKSSSRVQNAAGDIVSLRDVIESDKSTLLGEAVAKRFGELPFLFKVLCAAQPLSIQVHPNKHNSEIGFAKENAAGIPMDAAERNYKDPNHKPELVFALTPFLAMNAFREFSEIVSLLQPVAGAHPAIAHFLQQPDAERLSELFASLLNMQGEEKSRALAILKSALDSQQGEPWQTIRLISEFYPEDSGLFSPLLLNVVKLNPGEAMFLFAETPHAYLQGVALEVMANSDNVLRAGLTPKYIDIPELVANVKFEAKPANQLLTQPVKQGAELDFPIPVDDFAFSLHDLSDKETTISQQSAAILFCVEGDATLWKGSQQLQLKPGESAFIAANESPVTVKGHGRLARVYNKL

SEQ ID NO: 33 - protein, 456 aa, Escherichia coli K12 MG1655
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFKALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTAAGGTPVMSKTGHAFIKERMRKEDAIYGGEMSAHHYFRDFAYCDSGMIPWLLVAELVCLKDKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 34 - protein, 478 aa, Escherichia coli K12 MG1655
MAQSKLYPVVMAGGSGSRLWPLSRVLYPKQFLCLKGDLTMLQTTICRLNGVECESPVVICNEQHRFIVAEQLRQLNKLTENIILEPAGRNTAPAIALAALAAKRHSPESDPLMLVLAADHVIADEDAFRAAVRNAMPYAEAGKLVTFGIVPDLPETGYGYIRRGEVSAGEQDMVAFEVAQFVEKPNLETAQAYVASGEYYWNSGMFLFRAGRYLEELKKYRPDILDACEKAMSAVDPDLNFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 35 - protein, 373 aa, Escherichia coli K12 MG1655 [sequence omitted]

SEQ ID NO: 36 - protein, 321 aa, Escherichia coli K12 MG1655
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 37 - protein, 438 aa, Escherichia coli K12 MG1655
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 38 - protein, 949 aa, Bacteroides fragilis NCTC 9343
MQKLLSLPSNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILMKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAVSVFPMVNSVEELGLVLRWMLSEPELEEGKNIWLRSEHFSADEISAGANLKRLYAQREEFRKGNWKALAVNHEKSVFYQLDLADAAEDFVRLGLDMPELLPEDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 39 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 40 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 41 - protein, 349 aa, Neisseria meningitidis
MQNNNEFKIGNRSVGYNHEPLIICEIGINHEGSLKTAFEMVDAAYNAGAEVVKHQTHIVEDEMSDEAKQVIPGNADVSIYEIMERCALNEEDEIKLKEYVESKGMIFISTPFSRAAALRLQRMDIPAYKIGSGECNNYPLIKLVASFGKPIILSTGMNSIESIKKSVEIIREAGVPYALLHCTNIYPTPYEDVRLGGMNDLSEAFPDAIIGLSDHTLDNYACLGAVALGGSILERHFTDRMDRPGPDIVCSMNPDTFKELKQGAHALKLARGGKKDTIIAGEKPTKDFAFASVVADKDIKKGELLSGDNLWVKRPGNGDFSVNEYETLFGKVAACNIRKGAQIKKTDIE

SEQ ID NO: 42 - protein, 223 aa, Pasteurella multocida
MTNIAIIPARAGSKGIPDKNLQPVGGHSLIGRAILAAKNADVFDMIVVTSDGDNILREAEKYGALALKRPAELAQDNSRTIDAILHALESLNIREGTCTLLQPTSPLRDHLDIKNAMDMYVNGGVHSVVSACECEHHPYKAFALSKDHEVLPVREIADFEAARQTLPKMYRANGAIYINDIAQLLKEKYFFIPPLKFYLMPTYRSVDIDVKQDLELAEILSNK

SEQ ID NO: 43 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG

SEQ ID NO: 44 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 45 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 46 - protein, 498 aa, Photobacterium sp. JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 47 - protein, 188 aa, Escherichia coli K12 MG1655
MYERYAGLIFDMDGTILDTEPTHRKAWREVLGHYGLQYDIQAMIALNGSPTWRIAQAIIELNQADLDPHALAREKTEAVRSMLLDSVEPLPLVDVVKSWHGRRPMAVGTGSESAIAEALLAHLGLRHYFDAVVAADHVKHHKPAPDTFLLCAQRMGVQPTQCVVFEDADFGIQAARAAGMDAVDVRLL

SEQ ID NO: 48 - protein, 373 aa, Campylobacter jejuni
MVKKILFITGSRADYSKIKSLMYRVQNSSEFELYIFATGMHLSKNFGYTVKELYKNGFKNIYEFINYDKYYQTDKALATTIDGFSRYANELKPDLIVVHGDRIEPLAAAIVGALNNILVAHIEGGEISGTIDDSLRHAISKLAHIHLVNDEFAKRRLMQLGEDEKSIFIIGSPDLELLNDNKISLSEAKKYYDINYENYALLMFHPVTTEITSIKNQADNLVKALIQSNKNYIVIYPNNDLGFELILQSYEEFKNNPRFKLFPSLRFEYFITLLKNADFIIGNSSCILKEALYLKTAGILVGSRQNGRLGNENTLKVNANSDEILKAINTIHKKQDLFSAKLEILDSSKLFFEYLQSGDFFKLSTQKVFKDIK

SEQ ID NO: 49 - protein, 415 aa, Escherichia coli W
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 50 - protein, 301 aa, Zymomonas mobilis
MKNDKKIYGCIEGGGTKFMLALIDSDRKMLAVERVPTTTPEETLGKSVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 51 - protein, 504 aa, Bifidobacterium adolescentis [sequence omitted]

SEQ ID NO: 52 - protein, 417 aa, Escherichia coli K12 MG1655 [sequence omitted]

SEQ ID NO: 53 - protein, 410 aa, Cronobacter sakazakii strain MOD1_LR753
MQTHANRTGRLGRQALLFPLCLVLYEFSTYIGNDMIQPGMLAVVEQYQAGVEWVPTSMTAYLAGGMFLQWLLGPLSDRIGRRPVMLAGVAWFIVTCLATLLARDIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALIIGNLVLAKLTARRSVRSLIVMGGWPMMFGLALAALATVISSHAYLWMTAGLSIYAFGIGIANAGLVRLTLFASDISKGTVSAAMGMLQMTIFTVGIEISKHAWLGGGNALFNLFNFASGLLWLGLMVIFLKDKTVGTRPEA

SEQ ID NO: 54 - protein, 411 aa, Franconibacter pulveris LMG 24059
MQNKTMTKKRLGRQALLFPLCLVLYEFSTYIGNDMIQPGMLAVVEQYQAGMEWVPTSMTAYLAGGMFLQWLLGPLSDRIGRRPVMLTGVVWFIVTCLATLLAQNIEQFTALRFLQGISLCFIGAVGYAAIQESFEEAVCIKITALMANVALIAPLLGPLVGAAWVHMAPWEMMFVLFAVLAAIAYYGLWRAMPETATRLGEKLSLKELGRDYKAVMKNARFVAGALATGFVSLPLLAWIAQSPVIIISGEQLSSYEYGLLQVPIFGALIAGNLALAKLTSRRTVRSLIILGGWPITLGLVIAALATLLSSHAYLWMTAGLSVYAFGIGLANAGLVRLTLFASDISKGTVSAAMGMLQMLIFTVGIEVSKHAYLAGGNGLFSLFNLLNGLLWLGMMVIFLKDKTVGNSLQPQ

SEQ ID NO: 55 - protein, 410 aa, Enterobacter hormaechei strain 017
MINRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFVLFAALAAISFFGLHRAMPETATRLGEKLSLKELGRDYKAVLKNGRFVAGALATGFVSLPLLAWIAQSPVIIISGEQLSSYEYGLLQVPIFGALIIGNLVLARLTSRRTVRSLIIMGGWPIAAGLIIAAVATVASSHAYLWMTAGLSIYAFGIGVANAGLVRLTLFASEMSKGTVSAAMGMLQMLIFTVGIEVSKHAYAFGGNGLFSLFNLANGVLWVGLMVVFLKDKRVGNALQP

SEQ ID NO: 56 - protein, 410 aa, Citrobacter koseri strain NCTC10771
MQNLSQTGVRLGRQALLFPLCLVLYEFSTYIGNDMIQPGMLAVVEQYQAGIDWVPTSMTAYLAGGMFLQWLLGPLSDRIGRRPVMLAGVIWFIVTCLATLLAQNIEQFTLLRFLQGVSLCFIGAVGYAAIQESFEEAVCIKITALMANVALIAPLLGPLVGAAWVHVLPWEEMFVLFAVLAAIAFVGLQRAMPETATRLGEKLSLKELGRDYKLVLKNVRFVAGALALGFVSLPLLAWIAQSPIIIISGEQLSSYEYGLLQVPVFGALIAGNLVLARLTSRRTVRALIIMGGWPIVAGLLIAAAATVVSSHAYLWMTAGLSVYAFGIGVANAGLVRLTLFASDMSKGTVSAAMGMLQMLIFTVGIEVSKHAYLSGGNGLFSLFNLANGILWLLLMVIFLKDKRVGDSREG

SEQ ID NO: 57 - protein, 410 aa, Salmonella enterica subsp. arizonae serovar 41:z4,z23:- strain TAMU30EF
MHNRLQSGVRLGRQALLFPLCLVLYEFSTYIGNDMIQPGMLAVVEQYQAGLDWVPTSMTAYLAGGMFLQWLLGPLSDRIGRRPVMLAGVVWFIVTCLATLLAKNIEQFTFLRFLQGISLCFIGAVGYAAIQESFEEAVCIKITALMANVALIAPLLGPLVGAAWVHVLPWEGMFILFAALAAIAFFGLQRAMPETATRRGETLSFKALGRDYRLVIKNRRFVAGALALGFVSLPLLAWIAQSPIIIISGEQLSSYEYGLLQVPVFGALIAGNLVLARLTSRRTVRSLIVMGGWPIVAGLVIAAAATVVSSHAYLWMTAGLSVYAFGIGLANAGLVRLTLFSSDMSKGTVSAAMGMLQMLIFTVGIEVSKHAWLSGGNGLFSLFNLANGILWLLLMLVFLKDKRTGNLHTV

SEQ ID NO: 58 - protein, 408 aa, Shigella flexneri strain 585219
MQNKLASGARLGRQALLFPLCLVLYEFSTYIGNDMIQPGMLAVVEQYQAGIDWVPTSMTAYLAGGMFLQWLLGPLSDRIGRRPVMLAGVLWFIVTCLAILLAQNIEQFTMLRFLQGISLCFIGAVGYAAIQESFEEAVCIKITALMVNVALIAPLLGPLVGAAWIHVLPWEGMFVLFAALAAISFFGLQRAMPETATRIGEKLSLKELGRDYKLVLKNGRFVAGALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 59 - protein, 410 aa, Yokenella regensburgei strain UMB0819
MLNRSSSGSRLGRQALLFPLCLVLYEFSTYIGNDMIQPGMLAVVAQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFVLFAVLAAIAFFGLHRAMPETATRLGEKLSLKELGRDYKAVLQNGRFVAGALATGFVSLPLLAWIAQSPVIIISGEKLSSYEYGLLQVPIFGALIIGNLVLARLTSRRTVRSLIIMGGWPIAAGLILAAVATVASSHAYLWMTAGLSIYAFGIGVANAGLVRLTLFASEMSKGTVSAAMGMLQMLIFTVGIEVSKHAYALGGNGLFSLFNLANGVLWVGLMVVFLKDKRVGNALQP

SEQ ID NO: 60 - protein, 410 aa, Escherichia coli strain AMC_967
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIITCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMAAGLSLYAFGIGLANAGLVRLTLFASDMSKGTVSAAMGMLQMLIFTVGIEISKHAWLNGGNGLFNLFNLVNGILWLLLMVIFLKDKQMGNSHEG

SEQ ID NO: 61 - protein, 410 aa, Klebsiella pneumoniae VAKPC309
MQNYSLSGRRLGRQALLFPLCLVLYEFSTYIGNDMIQPGMLAVVQEFQVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHVLPWEMMFVLFAVLAAISFFGLQRAMPETATRLGEKLSVKELGRDYRLVLKNLRFVAGALATGFVSLPLLAWIAQSPVIIISGEQATSYEYGMLQVPIFGALIAGNLVLARLTARRTVRSLIIMGGWPIMFGLILSAAATVVSSHAYLWMTAGLSFYAFGIGLANAGLVRLTLFASEMSKGTVSAAMGMLQMLIFTVGIELSKHAYELGGNGLFSLFNLLGGVLWLGLMIYFLKDKSVGNSQQG

SEQ ID NO: 62 - protein, 411 aa, Klebsiella oxytoca strain 4928STDY7071490
MHNSSLSGKRLGRQALLFPLCLVLYEFATYIGNDMIQPGMLSVVEEFGVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHILPWEMMFVLFAVLAAIAFFGLQRAMPETATRMGEKLSVKELGRDYREVLKNLRFVAGALATGFVSLPLLAWIAQSPVIIISGEQATSYEYGLLQVPIFGALIAGNLVLARQTSRKTVRSLIILGGWPIMLGLLISAVATVASTHAYLWMTAGLSVYAFGIGLANAGLVRLTLFASEMSKGTVSAAMGMLQMLIFTVGIEVSKHAYESGGSGLFSLLNLLSGVLWLAMMVYFLKDKRVGSSLEPQ

SEQ ID NO: 63 - protein, 411 aa, Klebsiella michiganensis strain A2
MHNSSLSGKRLGRQALLFPLCLVLYEFATYIGNDMIQPGMLSVVEEFGVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHLLLWEMMFVLFAVLAAIAFFGLQKAMPETATRMGEKLSVKELGRDYREVLKNLRFVAGALATGFVSLPLLAWIAQSPVIIISGEQATSYEYGLLQVPIFGALIAGNLVLARQTSRKTVRSLIILGGWPIMIGLLISAVATVASTHAYLWMTAGLSVYAFGIGLANAGLVRLTLFASEMSKGTVSAAMGMLQMLIFTVGIEVSKHAYEAGGSGLFSLLNLLSGVLWLAMTVYFLKDKRVGSSLEPQ

SEQ ID NO: 64 - protein, 409 aa, Pluralibacter gergoviae strain FDAARGOS_186
MQNHALSGKRLGRSALLFPLCLVLYEFSTYIGNDMIQPGMLAVVAEFQASTEWVPTSMTAYLAGGMFLQWLLGPLSDRVGRRPVMLAGVVWFILTCLATLLVQNIEQFTLLRFLQGCGLCFVGAVGYAAIQESYEEAVSIKITALMANVALIAPLLGPLVGAAWVHAASWESMFVLFAALAAVAFVGLQRAMPETATRLGERLSLKELGHDYGLVLKNLRFVAGALATGFISLPLLAWIAQSPVIIISGEQASSYEYGMLQVPIFGALIAGNLVLAKLTARCSVRWLIIAGGWPAMTGLLVAALATVVSSHAYLWMTAGLSIYAFGIGISNAGLVRLTLFASEMSKGTVSSAMGVLQMLIFTVGIEISKHAYQLGGSGLFSLFNLLSGALWLGLIIYFLKDKRVGSGNR

SEQ ID NO: 65 - protein, 410 aa, Kluyvera ascorbata ATCC 33433
MDTSSSRKRLGRKAILFPLCLVLYEFSTYIGNDMIQPGMLAVVKEFNAGNEWVPTSMTAYLAGGMFLQWLLGPLSDRIGRRPVMLTGVIWFIVTCLATLLVNNIEQFTFLRFLQGVSLCFIGAVGYAAIQESFDEATCIKITALMANVALIAPLLGPLVGAAWIHVARWEGMFVLFAALSAIAFVGLFKSMPETATRIGETLSLKALGRDYREVLKNGRFVAGALATGFVSLPLLAWIAQSPVIIISGEHASSYEYGLLQVPIFGALILGNLVLARLTSRRTVRALIIAGGYPIMFGLVLAAAATVVSSHAYLWMTAGLSFYAFGIGLANAGLVRLTLFASDMSKGTVSAAMGMLQMLIFTVGIELSKHCYELGGIGLFSLFNLAGGLLWLGLMTMFLRDKAVGSGLQPQ

SEQ ID NO: 66 - protein, 409 aa, Enterobacter kobei
MQNHSLPGRRLGRQALLFPLCLVLYEFSTYIANDMIQPGMLAVVEQYNAGIEWVPTSMTAYLAGGMFLQWLLGPLSDRIGRRPVMLTGVVWFIVTCLATLLAQDIEQFTLLRFLQGVSLCFIGAVGYAAIQESFDEATCIKITALMANVALIAPLLGPLVGAAWVHAAPWEGMFVLFAVLAAIAFFGLHRAMPETATRLGEPLSLNALGRDYKAVLKNGRFVAGALATGFVSLPLLAWIAQSPIIIISAEGMSSYEYGLLQVPIFGALIIGNLVLARLTSRRTVRSLIIMGGGPIVAGLLVAAVATVASSHAYLWMTAGLSIYAFGIGLANAGLVRLTLFASDMSKGTVSAAMGMLQMAIFTVGIEVSKHAFLAGGNGLFSLFNLANGLIWLALMVVFLKDKTVGNALS

SEQ ID NO: 67 - protein, 409 aa, Lelliottia sp. WB101
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLAILLAQTIEQFTLLRFLQGVSLCFIGAVGYAAIQESFEEAVCIKITALMANVALIAPLLGPLVGAAWVHVAPWEGMFVLFAALAAISFFGLHRAMPETATRLGEKLSLKELGRDYKEVLRNGRFVAGALATGFVSLPLLAWIAQSPVIIISGEKLSSYEYGLLQVPVFGALIIGNLVLARLTSRRSVRSLIIMGGWPIVAGLVVAAVATVASSHAYLWMTAGLSIYAFGIGLANAGLVRLTLFASEMSKGTVSAAMGMLQMLIFTVGIEVSKHAYSFGGNGLFSLFNLANGVLWLGLMVMFLKDKRVGSALQ

SEQ ID NO: 68 - protein, 410 aa, Citrobacter freundii
MQNRLSSGARLGRQALLFPLCLVLYEFSTYIGNDMIQPGMLAVVEQYQAGLDWVPTSMTAYLAGGMFLQWLLGPLSDRVGRRPVMLTGVVWFIVTCLATLFAQNIEQFTFLRFLQGISLCFIGAVGYAAIQESFEEAVCIKITALMANVALIAPLLGPLVGAAWVHVLPWEGMFVLFAALAAIAFFGLQRAMPETATRLGEKLSIKELGKDYKLVLKNVRFVAGALALGFVSLPLLAWIAQSPIIIISGEHLSSYEYGLLQVPIFGALIAGNLVLARLTSRRTVRSLIIMGGWPISVGLIIAAAATVVSSHAYLWMTAGLSLYAFGIGVANAGLVRLTLFASEMSKGTVSAAMGMLQMLIFTVGIELSKHAYLLGGNGLFSLFNLASGVLWLILMVIFLKDKRVGNSREG

SEQ ID NO: 69 - protein, 410 aa, Salmonella enterica subsp. Salamae
MHNRLQSGGRLGRQALLFPLCLVLYEFSTYIGNDMIQPGMLAVVAQYQASLDWVPTSMTAYLAGGMFLQWLLGPLSDRIGRRPVMLAGVVWFIVTCLATLLAKNIEQFTFLRFLQGISLCFIGAVGYAAIQESFEEAVCIKITALMANVALIAPLLGPLVGAAWVHVLPWEGMFILFAVLAAIAFFGLQRAMPETATRRGETLSFKVLGRDYRLVIKNRRFVAGALALGFVSLPLLAWIAQSPIIIISGEQLSSYEYGLLQVPVFGALIAGNLVLARLTSRRTVRSLIVMGGWPIVAGLIIAAAATVVSSHAYLWMTAGLSVYAFGIGLANAGLVRLTLFSSDMSKGTVSAAMGMLQMLIFTVGIEVSKHAWLSGGNGLFSLFNLANGILWLLLMLVFLKDKRTGDSQTG

SEQ ID NO: 70 - protein, 408 aa, Shigella flexneri
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISFCFIGAVGYAAIQESFEEAVCIKITALMANVALIAPLLGPLVGAAWIHVLPWEGMFVLFAALAAISFFGLQRAMPETAMRIGEKLSLKELGRDYKLVLKNGRFVAGALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHGG

SEQ ID NO: 71 - protein, 410 aa, Cronobacter sakazakii strain MOD1_LR634
MQTHANRTGRLGRQALLFPLCLVLYEFSTYIGNDMIQPGMLAVVEQYQAGVEWVPTSMTAYLAGGMFLQWLLGPLSDRIGRRPVMLAGVAWFIVACLATLLAQNIEQFTVLRFLQGISLCFIGAVGYAAIQESFEEAVCIKITALMANVALIAPLLGPLVGAAWVHAAPWEMMFVLFAVLATISFFGLWRAMPETATRLGEKLSLRELGRDYKAVLKNLRFVSGALAIGFVSLPLLAWIAQSPVIIISGEQMSTYEYGLLQVPIFGALIVGNLVLAKLTARRSVRSLIVMGGWPMMFGLALAALATVISSHAYLWMTAGLSIYAFGIGIANAGLVRLTLFASDISKGTVSAAMGMLQMTIFTVGIEISKHAWLGGGNALFNLFNFASGLLWLGLMVIFLKDKTVGTRPEA

SEQ ID NO: 72 - protein, 410 aa, Cronobacter condimenti strain s37
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFISLPLLAWIAQSPVIIISGEQMSTYEYGLLQVPIFGALIVGNLLLAKLTARRSVRSLIIMGGWPMMIGLALAALATVLSSHAYLWMTAGLSIYAFGIGIANAGLVRLTLFASDISKGTVSAAMGMLQMTIFTVGIEVSKHAWIIGGNALFNLFSFASGLLWLGLMVIFLKDKTVGASRGG

SEQ ID NO: 73 - protein, 410 aa, Cronobacter sp. EKM102R
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALILGNLVLAKLTARRAVRSLIVMGGWPIVAGLALAALATVISSHSYLWMTAGLSIYAFGIGIANAGLVRLTLFASDISKGTVSAAMGMLQMTIFTVGIEVSKHAWIGGGNALFNLFNFASGLLWLGLMVIFLKDKTVGTRPEG

SEQ ID NO: 74 - protein, 410 aa, Cronobacter universalis NCTC 9529
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALIIGNLVLAKLTARRSVRSLIVMGGWPMMAGLALAALATVISSHAYLWMTAGLSIYAFGIGIANAGLVRLTLFASDISKGTVSAAMGMLQMTIFTVGIEVSKHAWLGGGNALFNLFNFASGLLWLGLMAIFLKDKTVGTRPEG

SEQ ID NO: 75 - protein, 411 aa, Salmonella enterica strain 413_SENT
MQNNSLTKKRLGRQALLFPLCLVLYEFSTYIGNDMIQPGMLAVVEQYQAGVEWVPTSMTAYLAGGMFLQWLLGPLSDRIGRRPVMLTGVVWFIVTCLATLLAQNIEQFTALRFLQGISLCFIGAVGYAAIQESFEEAVCIKITALMANVALIAPLLGPLVGAAWVHVAPWEMMFVLFAVLAAISWYGLWRAMPETATRLGEKLSLGELGRDYKAVMKNARFVAGALATGFVSLPLLAWIAQSPVIIISGEQLSSYEYGLLQVPIFGALIAGNLVLAKLTSRRTVRSLIILGGWPIAFGLAMAALATLFSAHAYLWMTAGLSVYAFGIGLANAGLVRLTLFASDISKGTVSAAMGMLQMLIFTVGIEVSKHAYLAGGNGLFSLFNLLNGLLWVAMMVIFLKDKTVGNSLQPQ

SEQ ID NO: 76 - protein, 393 aa, Klebsiella pneumoniae subsp. pneumoniae strain NCTC11695
MQNYSLSGRRLGRQALLFPLCLVLYEFSTYIGNDMIQPGMLAVVQEFQVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHVLPWEMMFVLFAVLAAISFVGLQRAMPETATRLGEKLSVKELGRDYRLVLKNLRFVAGALATGFVSLPLLAWIAQSPVIIISGEQATSYEYGMLQVPIFGALIAGNLVLARLTSRRTVRSLIIMGGWPIMFGLILSAAATVVSSHAYLWMTAGLSFYAFGIGLANAGLVRLTLFASEMSKGTVSAAMGMLQMLIFTVGIELSKHAYELGATACSACLTCWAEYCGWG

SEQ ID NO: 77 - protein, 410 aa, Klebsiella aerogenes strain 4928STDY7071344
MQNYSLSGKRLGRQALLFPLCLVLYEFSTYIGNDMIQPGMLAVVQEFQVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHMLPWEMMFVLFAVLAAISFIGLQRAMPETATRMGEKLSVKELGRDYRLVLKNLRFVAGALATGFVSLPLLAWIAQSPVIIISGEQATSYEYGMLQVPIFGALIAGNLVLARLTSRRSVRSLIIMGGWPIMFGLLLSAAATVVSSHAYLWMTAGLSFYAFGIGLANAGLVRLTLFASEMSKGTVSAAMGMLQMLIFTVGIELSKHAYELGGNGLFSLFNLLGGVLWLGLIIYFLKDKSVGSSQQA

SEQ ID NO: 78 - protein, 412 aa, Raoultella planticola strain FDAARGOS_283
MQNYSLSGKRLGRQALLFPLCLVLYEFSTYIGNDMIQPGMLSVVEEFQVGNEWVPTSMTAYLAGGMFLQWLLGPLSDRIGRRPVMLTGVVWFIVTCLATLLAQTIEQFTLLRFLQGISLCFIGAVGYAAIQESFEEAMCIKITALMANVALIAPLLGPLVGAAWVHILPWEMMFVLFAVLAAIAFVGLQRAMPETATRMGEKLSMKELGRDYGLVLKNLRFVAGALATGFVSLPLLAWIAQSPVIIISGEKATSYEYGLLQVPIFGALIAGNLVLARLTSRRTVRSLIILGGWPIMFGLLLSAVATVVSTHAYLWMTAGLSVYAFGIGLANAGLVRLTLFASDMSKGTVSAAMGMLQMLIFTVGIEVSKHAYELGGSGVFSLFNLLSGVMWLAMIVYFLKDKSVGNSNDPQA

SEQ ID NO: 79 - protein, 411 aa, Klebsiella sp. 2680
MHNYSLAGKRLGRQALLFPLCLVLYEFSTYIGNDMIQPGMLSVVDAFGVGNEWVPTSMTAYLAGGMFLQWLLGPLSDRIGRRPVMLTGVVWFIATCLATLLAQTIEQFTLLRFLQGISLCFIGAVGYAAIQESFEEAVCIKITALMANVALIAPLLGPLVGAAWVHFLPWEMMFVLFAVLATIAFVGLQRAMPETATRQGEKLSVKALGRDYRDVLKNRRFVAGALATGFVSLPLLAWIAQSPVIIISGEQATSYEYGLLQVPIFGALIAGNLVLARQTSRKTVRSLIILGGWPIITGLLISAAATVVSAHAYLWMTAGLSVYAFGIGLANAGLVRLTLFASDMSKGTVSAAMGMLQMLIFTVGIEVSKHAWEFGGSGLFSLLNLLSGALWLVMTAYFLKDKRAGNSREPQ

SEQ ID NO: 80 - protein, 411 aa, Kluyvera georgiana strain HRGM_Genome_0064
MDTLSSSRKRLGRKAILFPLCLVLYEFSTYIGNDMIQPGMLAVVKEFNAGDEWVPTSMTAYLAGGMFLQWLLGPLSDRIGRRPVMLTGVVWFIVTCLATLLVQNIEQFTFLRFLQGVSLCFIGAVGYAAIQESYDEATCIKITALMANVALIAPLLGPLVGAAWIHVARWESMFVLFAALAALAYIGLHKEMPETATRIGEKLSLKELGRDYREVLKNGRFVAGALATGFVSLPLLAWIAQSPVIIISGEGASSYEYGLLQVPIFGALILGNLVLARLTSRRTVRALIVAGGYPIMFGLILAAAATVVSSHAYLWMTAGLSFYAFGIGLANAGLVRLTLFASEMSKGTVSAAMGMLQMLIFTVGIELSKHMYLLGGIGLFSLFNLAGGLLWLGLMVMFLRDKTVGSGLQPQ

SEQ ID NO: 81 - protein, 411 aa, Kluyvera intermedia strain NCTC12125
MDTSSSSRKRLGRKAILFPLCLVLYEFSTYIANDMIQPGMLAVVKEFNAGDEWVPTSMTAYLAGGMFLQWLLGPLSDRIGRRPVMLTGVIWFIVTCLATLMVQNIEQFTFLRFLQGVSLCFIGAVGYAAIQESFDEATCIKITALMANVALIAPLLGPLVGAAWIHLARWESMFVLFAVLAAIAYVGLFKAMPETATRLGEKLSLKTLGQDYREVLKNGRFVAGALATGFVSLPLLAWIAQSPVIIISGENASSYEYGLLQVPIFGALILGNLVLARLTSRRTVRALIIAGGYPIMFGLILAAAATVVSSHAYLWMTAGLSFYAFGIGLANAGLVRLTLFASDMSKGTVSAAMGMLQMLIFTVGIELSKHSYQMGGIGLFSLFNLAGGVLWLVLMVMFLRDKTVGSGLQPE

SEQ ID NO: 82 - protein, 431 aa, Salmonella enterica strain 2014K-0203
MHNRLQSGVRLGRQALLFPLCLVLYEFSTYIGNDMIQPGMLAVVEQYQVSLDWVPTSMTAYLAGGMFLQWLLGPLSDRIGRRPVMLAGIVWFIATCLATLLAKNIEQFTFLRFLQGISLCFIGAVGYAAIQESFEEAVCIKITALMANVALIAPLLGPLVGAAWVHVLPWEGMFVLFAALAAIAFFGLQRAMPETATRRGETLSLKALGRDYRLVIKNRRFVAGALALGFVSLPLLAWIAQSPIIIISGEQLSSYEYGLLQVPVFGALIAGNLVLARLTSRRTVRSLIVMGGWPIVVGLIIAAAAAVVSSHAYLWMMAGLSVYAFGIGLANAGLVRLTLFSSEMSKGTVSAAMGMLQMLIFTVGIEVSKHAWLSGGNELFSLFNLANGILWLLLMLVFLKDKRAGIRKQAKRAAMPDKPSVIRHREASAKR

SEQ ID NO: 83 - protein, 416 aa, Salmonella bongori strain 85-0051
MHNRTQSGARLGRQALLFPLCLVLYEFSTYIGNDMIQPGMLAVVEQYQAGLDWVPTSMTAYLAGGMFLQWLLGPLSDRIGRRPVMLAGVIWFIVTCLATLLAKNIEQFTFLRFLQGISLCFIGAVGYAAIQESFEEAVCIKITALMANVALIAPLLGPLVGAAWVHVLPWEGMFILFAALAAIAFFGLQRAMPETATRRGETLSLKDLGRDYQRVIKNRRFVAGALALGFVSLPLLAWIAQSPIIIISGEQLSSYEYGLLQVPVFGALIAGNLVLARLTSRRTVRSLIVMGGWPIVAGLIIAAAATVVSSHAYLWMTAGLSIYAFGIGLANAGLVRLTLFASDMSKGTVSAAMGMLQMLIFTVGIEVSKHAWLSGGNGLFSLFNLANGIFWLLLMLVFLKNKRTGDSQTDPESGYA

SEQ ID NO: 84 - protein, 412 aa, Salmonella enterica subsp. enterica serovar Hillingdon strain S01-0588
MVCRIVYNQAAGWDVRRCFFLSVWCCTSFSTYIGNDMIQPGMLAVVEQYQAGLDWVPTSMTAYLAGGMFLQWLLGPLSDRIGRRPVMLAGVVWFIVTCLATLLAKNIEQFTFLRFLQGISLCFIGAVGYAAIQESFEEAVCIKITALMANVALIAPLLGPLVGAAWVHVLPWEGMFILFAALAAIAFFGLQRAMPETATRRGETLSFKALGRDYRLVIKNRRFVAGALALGFVSLPLLAWIAQSPIIIISGEQLSSYEYGLLQVPVFGALIAGNLVLARLTSRRTVRSLIVMGGWPIVAGLIIAAAATVVSSHAYLWMTAGLSVYAFGIGLANAGLVRLTLFSSDMSKGTVSAAMGMLQMLIFTVGIEVSKHAWLSGGNGLFSLFNLANGILWLLLMLVFLKDKRTGNLQTV

SEQ ID NO: 85 - protein, 410 aa, Citrobacter youngae strain TE1
MQNRLSSGARLGRQALLFPLCLVLYEFSTYIANDMIQPGMLAVVEQYQAGIDWVPTSMTAYLAGGMFLQWLLGPLSDRIGRRPVMLAGVVWFIVTCLATLLAQNIEQFTFLRFLQGISLCFIGAVGYAAIQESFEEAVCIKITALMANVALIAPLLGPLVGAAWVHVLPWEGMFVLFAVLAAFAFFGLQRAMPETATRIGEKLSLKELWRDYKLVLKNVRFVAGALALGFVSLPLLAWIAQSPIIIISGEQLSSYEYGLLQVPVFGALIAGNLVLARLTARRTVRSLIIMGGWPIMLGLILAAAATVASSHAYLWMTAGLSLYAFGIGVANAGLVRLTLFASDMSKGTVSAAMGMLQMLIFTVGIEVSKYAYLGGGNGLFSLFNLANGILWLLLMFIFLKDKQVGASRGG

SEQ ID NO: 86 - protein, 410 aa, Citrobacter sp. RHB21-C01
MQNRLQQGARLGRQALLFPLCLVLYEFSTYIGNDMIQPGMLAVVAQYQAGIDWVPTSMTAYLAGGMFLQWLLGPLSDRIGRRPVMLAGVVWFIVTCLATLLAQTIEQFTLLRFLQGISLCFIGAVGYAAIQESFEEAVCIKITALMANVALIAPLLGPLVGAAWVHVLPWEGMFVLFAVLAAMAFFGLQRAMPETATRIGEPLSLKALGRDYQQVMKNGRFVAGALALGFVSLPLLAWIAQSPIIIISGEQLSSYQYGLLQVPIFGALIAGNLVLARLTSRRTVRSLIIIGGWPIVLGLILAAAATVVSSHAYLWMTAGLSVYAFGIGLANAGLVRLTLFASDMSKGTVSAAMGMLQMLIFTLGIEVSKYAYLSGGNGLFSLFNLTNGILWLLLMVIFLKDKQVGNTREG

SEQ ID NO: 87 - protein, 410 aa, Citrobacter werkmanii strain MGYG-HGUT-02535
MHNRLSSGARLGRQALLFPLCLVLYEFSTYIGNDMIQPGMLAVVEQYQAGLDWVPTSMTAYLAGGMFLQWLLGPLSDRIGRRPVMLTGVVWFIVTCLATLFAQNIEQFTFLRFLQGISLCFIGAVGYAAIQESFEEAVCIKITALMANVALIAPLLGPLVGAAWVHVLPWEGMFVLFAVLAAIAFVGLQRAMPETATRLGETLSIKELGKDYKLVLKNVRFVAGALALGFVSLPLLAWIAQSPIIIISGEQLSSYEYGLLQVPIFGALIAGNLVLARLTARRTVRSLIIMGGWPIAVGLIIAAAATVVSSHAYLWMTAGLSVYAFGIGVANAGLVRLTLFASDMSKGTVSAAMGMLQMLIFTVGIELSKHAYLLGGNGLFSLFNLASGILWLALMFIFLKDKRVGDSREG

SEQ ID NO: 88 - protein, 409 aa, Enterobacteriaceae bacterium UBA3109
MQNHSLSGGRLGRQALLFPLCLVLYEFSTYIANDMIQPGMLAVVEQYHAGIEWVPTSMTAYLAGGMFLQWLLGPLSDRIGRRPVMLTGVVWFILTCLATLLAQNIEQFTILRFLQGVSLCFIGAVGYAAIQESFDEATCIKITALMANVALIAPLLGPLVGAAWVHAAPWEGMFILFAVLAAIAFFGLQRAMPETATRLGEPLSLKALGRDYKAVLKNVRFVAGALATGFVSLPLLAWIAQSPIIIISGEGMSSYEYGLLQVPIFGALIIGNLVLARLTSRRTVRALIIFGGWPIVFGLALAAVATVVSSHAYLWMTAGLSLYAFGIGLANAGLVRLTLFASDMSKGTVSAAMGMLQMAIFTVGIELSKHAYLSGGSGLFSLFNLVNGVLWVALMVVFLRDKSVGNALS

SEQ ID NO: 89 - protein, 409 aa, Enterobacteriaceae bacterium UBA6698
MPNHALPGRRLGRQALLFPLCLVLYEFSTYIANDMIQPGMLAVVEQYNAGIEWVPTSMTAYLAGGMFLQWLLGPLSDRIGRRPVMLTGVVWFIVTCLATLLAQDIEQFTILRFLQGVSLCFIGAVGYAAIQESFDEATCIKITALMANVALIAPLLGPLVGAAWVHAAPWEGMFVLFAVLAAIAFFGLHRAMPETATRLGEPLSLNALGRDYKAVMKNGRFVAGALATGFVSLPLLAWIAQSPIIIISAEGMSSYEYGLLQVPIFGALIIGNLVLARLTSRRTVRSLIIMGGGPIVAGLLLAAVATVASSHAYLWMTAGLSIYAFGIGLANAGLVRLTLFASDMSKGTVSAAMGMLQMAIFTVGIEVSKHAFLAGGNGLFSLFNLANGLIWLVLMVVFLKDKSVGNALS

SEQ ID NO: 90 - protein, 411 aa, Leclercia sp. 4-9-1-25
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLAILLAQNIEQFTFLRFLQGVSLCFIGAVGYAAIQESFEEAVCIKITALMANVALIAPLLGPLVGAAWVHVAPWEGMFILFALLAAISFFGLWKAMPETATRLGETLSMRELGRDYKAVMKNVRFVAGALATGFVSLPLLAWIAQSPVIIISGEQLSSYEYGLLQVPIFGALIIGNLVLARLTSRRTVRSLIIMGGWPITLGLLLAAVATVVSSHAYLWMTAGLSLYAFGIGLANAGLVRLTLFASDMSKGTVSAAMGMLQMLIFTVGIEVSKHAFSFGGNGLFSLFNLANGLLWLVLMVIFLKDKRVGNALQPD

SEQ ID NO: 91 - protein, 411 aa, Lelliottia amnigena strain TZW14
MLNRSSSSGHRLGRQALLFPLCLVLYEFSTYIANDMIQPGMLAVVAQYNAGIEWVPTSMTAYLAGGMFLQWLLGPLSDRIGRRPVMLTGVVWFIVTCLAILLAQTIEQFMVLRFLQGVSLCFIGAVGYAAIQESFEEAVCIKITALMANVALIAPLLGPLVGAAWVHVAPWEGMFILFALLAAISWYGLHRAMPETATRLGEKLSLKELGRDYKEVLKNVRFVAGALATGFVSLPLLAWIAQSPVIIISGEKLSSYEYGLLQVPIFGALIIGNLVLARLTSRRSVRSLIILGGWPIAGGLILAAVATVVSSHAYLWMTAGLSIYAFGIGLANAGLVRLTLFASEMSKGTVSAAMGMLQMLIFTVGIEVSKHAYIIGGNGLFSLFNLANGVLWVGLMVIFLKNKRVGNALQP

SEQ ID NO: 92 - protein, 409 aa, Lelliottia aquatilis strain TZW17
MLNRSSSGNRLGRQALLFPLCLVLYEFSTYIGNDMIQPGMLAVVEQFNAGIEWVPTSMTAYLAGGMFLQWLLGPLSDRIGRRPVMLTGVVWFIVTCLAILLAQTIEQFTILRFLQGVSLCFIGAVGYAAIQESFEEAVCIKITALMANVALIAPLLGPLVGAAWVHVAPWEGMFVLFALLAAISFFGLHRAMPETATRLGEKLSLKELGRDYKEVLRNVRFVAGALATGFVSLPLLAWIAQSPVIIISGEKLSSYEYGLLQVPIFGALIIGNLVLARLTSRRSVRSLIIMGGWPIMAGLVVAAVATVASSHAYLWMTAGLSIYAFGIGLANAGLVRLTLFASEMSKGTVSAAMGMLQMLIFTVGIEVSKHAYSFGGNGLFSLFNLANGILWLGLMVVFLKDKRVGSALQ

SEQ ID NO: 93 - protein, 410 aa, Enterobacter sp. RHBSTW-00901
MLNRSSSGTRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAMCIKITALMANVALIAPLLGPLVGAAWIHVAPWEGMFVLFAAFAAIAFFGLHRAMPETATRLGETLSLKELGRDYKLVLKNVRFVAGALALGFVSLPLLAWIAQSPVIIISGEKLSSYEYGMLQVPIFGALIIGNLTLARLTSRRTVRSLIIMGGWPIVAGLIMAAVATVASSHAYLWMTAGLSVYAFGIGLANAGLVRLTLFASDMSKGTVSAAMGMLQMLIFTVGIEVSKHAYAMGGNGLFSLFNLANGVLWLGLMVVFLKDKRVGNALQP

SEQ ID NO: 94 - protein, 410 aa, Enterobacter cloacae subsp. dissolvens strain GN05902
MLNRSSSGTRLGRQALLFPLCLVLYEFSTYIGNDMIQPGMLAVVAQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFVLFAVLAAIAFFGLHRAMPETATRLGETLSLKELGRDYKAVLKNGRFVAGALATGFVSLPLLAWIAQSPVIIISGEQLSSYEYGLLQVPIFGALIIGNLVLARLTSRRTVRSLIIMGGWPIAAGLTVAAVATVVSSHAYLWMTAGLSIYAFGIGVANAGLVRLTLFASEMSKGTVSAAMGMLQMLIFTVGIEVSKHTYALGGNGLFSLFNLANGVLWVVLMVMFLKDKRVGNALQP

SEQ ID NO: 95 - protein, 410 aa, Enterobacter mori strain JGM37
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFILFAALAAISFFGLHRAMPETATRLGEKLSLKELGRDYKEVLKNGRFVAGALATGFVSLPLLAWIAQSPVIIISGEKLSSYEYGLLQVPIFGALIAGNLVLARLTSRRTVRSLIIMGGWPIAAGLVMAAVATVASSHAYLWMTAGLSVYAFGIGVANAGLVRLTLFASEMSKGTVSAAMGMLQMLIFTVGIEVSKHAYAFGGNGLFSLFNLANGVLWVGLMVVFLKDKRIGSALQP

SEQ ID NO: 96 - protein, 410 aa, Kosakonia sacchari strain BO-1
MQNHTLTSQRLGRRALLFPLCLVLYEFSTYIANDMIQPGMLAVVEQYNAGIEWVPTSMTAYLAGGMFLQWLLGPLSDRIGRRPVMLTGVVWFIVTCLATLLAQDIEQFTLLRFLQGISLCFIGAVGYAAIQESFEEAVCIKITALMANVALIAPLLGPLVGAAWVHAAPWQGMFVLFALLAAFAFFGLQRAMPETATRLGEKLSFKALWRDYADVMKNLRFVAGALATGFVSLPLLAWIAQSPIIIISGEQLSSYEYGLLQVPIFGALIIGNLVLARLTSRRTVRALIIMGGWPIAAGLLLAAVATVASSHAYLWMTAGLSLYAFGIGLANAGLVRLTLFASEMSKGTVSAAMGMLQMFIFTVGIEISKHAWLSGGNGLFSLFNLANGVLWLGLMVVFLRDKRVGNSLEG

SEQ ID NO: 97 - protein, 410 aa, Cronobacter malonaticus strain MOD1-Md25g
MQTHANRTGRLGRQALLFPLCLVLYEFSTYIGNDMIQPGMLAVVEQYQAGVEWVPTSMTAYLAGGMFLQWLLGPLSDRIGRRPVMLAGVAWFIVTCLATLLAQNIEQFTVLRFLQGISLCFIGAVGYAAIQESFEEAVCIKITALMANVALIAPLLGPLVGAAWVHAAPWEMMFVLFAVLAAISFFGLWRAMPETATRLGEKLSLRELGRDYKAVLKNLRFVSGALAIGFVSLPLLAWIAQSPVIIISGEQMSTYEYGLLQVPIFGALIIGNLVLAKLTARRSVRSLIVMGGWPMMFGLALAALATVISSHAYLWMTAGLSIYAFGIGIANAGLVRLTLFASDISKGTVSAAMGMLQMTIFTVGIEVSKHAWIGGGNALFNLFNFASGLLWLGLMVIFLKDKTVGTRPEA

SEQ ID NO: 98 - protein, 410 aa, Cronobacter dublinensis 582
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQNIEQFTVLRFLQGISLCFIGAVGYAAIQESFEEAVCIKITALMANVALIAPLLGPLVGAAWVHAAPWEMMFVLFAVLAAISFFGLWRAMPETATRLGEKLSLRELGRDYKKVLKNLRFVSGALAIGFVSLPLLAWIAQSPVIIISGEQMSTYEYGLLQVPIFGALIIGNLVLAKLTARRSVRSLIVMGGWPIMAGLALAAFATVISSHAYLWMTAGLSIYAFGIGIANAGLVRLTLFASDISKGTVSAAMGMLQMTIFTVGIEVSKHAWTGGGNGLFNLFNFASGLLWLGLTVIFLKDKTVGASREG